(19) United States
(12) Patent Application Publication
(10) Pub. No.: US 2024/0339216 A1
(43) Pub. Date: Oct. 10, 2024

(54) MYCOBIOME IN CANCER

(71) Applicant: The Regents of the University of California, Oakland, CA (US)

(72) Inventor: Gregory Poore, La Jolla, CA (US)

(21) Appl. No.: 18/579,487

(22) PCT Filed: Jul. 14, 2022
(86) PCT No.: PCT/US2022/037074

§ 371 Date: Jan. 15, 2024

Related U.S. Application Data
(60) Provisional application No. 63/221,504, filed on Jul. 14, 2021.

(51) Int. Cl. G16H 50/20; C12Q 1/6806; C12Q 1/6886; C12Q 1/6895; G16B 30/10; G16H 50/70

(52) U.S. Cl. CPC G16H 50/20; C12Q 1/6806; C12Q 1/6886; C12Q 1/6895; G16B 30/10; G16H 50/70; C12Q 2600/112; C12Q 2600/154

(57) ABSTRACT

Methods and systems are presented herein for predicting cancer of a subject through a combination of fungal and non-fungal features of a biological sample. Some embodiments, describe a method of predicting cancer of a subject from a combined fungal and non-fungal microbial presence of a biological sample by: detecting a fungal presence and a non-fungal microbial presence in a sample, removing contaminating fungal features of the fungal presence and contaminating non-fungal microbial features of the non-fungal microbial presence, and predicting a cancer of the subject by correlating the combined decontaminated fungal presence and the decontaminated non-fungal microbial presence to a known combined fungal presence and non-fungal microbial presence for one or more cancers.

### CROSS-REFERENCE

This application claims the benefit of U.S. Provisional Application No. 63/221,504 filed Jul. 14, 2021, which application is incorporated herein by reference.

### STATEMENT AS TO FEDERALLY SPONSORED RESEARCH

This invention was made with the support of the United States government under grant No. CA243480 awarded by the National Institutes of Health. The government has certain rights in the invention.

### SUMMARY

The invention provides methods and systems for determination of a fungal presence and/or abundance in a tissue sample, for detection and/or treatment of a cancer, as described herein.

Aspects of the disclosure, in some embodiments, describe a method of predicting cancer of a subject from a combined fungal and non-fungal microbial presence of a biological sample, comprising: (a) detecting a fungal presence and a non-fungal microbial presence in a biological sample from a subject; (b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and (c) predicting a cancer of the subject by correlating the combined decontaminated fungal presence and the decontaminated non-fungal microbial presence of the subject to a known combined fungal presence and non-fungal microbial presence for one or more cancers. In some embodiments, detecting comprises whole genome sequencing, shotgun sequencing, targeted sequencing, RNA sequencing, methylation sequencing, or any combination thereof. In some embodiments, the non-fungal microbial presence comprises bacteria, viruses, archaea, protists, or any combination thereof. In some embodiments, the non-fungal microbial presence comprises a non-fungal microbial abundance of the biological sample from the subject. In some embodiments, the fungal presence comprises a fungal abundance of the biological sample from the subject. In some embodiments, predicting the cancer further comprises predicting one or more cancers, one or more subtypes of cancer, the anatomic locations of one or more cancers, or any combination thereof in the subject. In some embodiments, predicting the cancer comprises predicting a stage of the cancer, cancer prognosis, a mutation status of the cancer, a future immunotherapy response of the cancer, an optimal therapy to treat the cancer, or any combination thereof for one or more subjects. In some embodiments, the cancer comprises a stage I or stage II cancer. In some embodiments, predicting the cancer comprises predicting a cancer type among one or more cancer types. In some embodiments, predicting the cancer comprises simultaneously discriminating among one or more cancer types to diagnose a specific cancer type of the subject. In some embodiments, the cancer comprises bone, breast, lung, colon, brain, skin, ovary, pancreas, or any combination thereof type of cancer. In some embodiments, the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some embodiments, the cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. In some embodiments, removing the contaminating non-fungal microbial features and the contaminating fungal features is completed by in silico decontamination. In some embodiments, removing the contaminating non-fungal microbial features and the contaminating fungal features is informed by experimental contamination controls. In some embodiments, predicting is conducted with a predictive model, wherein the predictive model comprises a machine learning model, regularized machine learning model, ensemble of machine learning models, or any combination thereof. In some embodiments, the predictive model comprises a random forest, neural network, naïve bayes, support vector machines, linear regression, k-nearest neighbors, k-means, decision tree, logistic regression, gradient boosting, or any combination thereof predictive model. In some embodiments, step (b) improves accuracy of the predictive model by at least 1%, at least 5%, at least 10%, at least 15%, or at least 20%. In some embodiments, step (b) is omitted. In some embodiments, the subject comprises a non-human mammal or a human subject. In some embodiments, the biological sample comprises a tissue sample, a liquid biopsy, whole blood biopsy, or any combination thereof samples. In some embodiments, the liquid biopsy comprises whole blood, red blood cells, plasma, white blood cells, saliva, urine, tears, breast milk, or any combination thereof. In some embodiments, the whole blood biopsy comprises plasma, white blood cells, red blood cells, platelets, or any combination thereof. In some embodiments, the fungal presence comprises an abundance of fungal DNA, RNA, methylation, proteins, or any combination thereof. In some embodiments, the non-fungal microbial presence comprises an abundance of non-fungal microbial DNA, RNA, methylation, proteins, or any combination thereof. In some embodiments, detecting the fungal presence and the non-fungal microbial presence in the biological sample comprises: (a) sequencing one or more nucleic acid molecules of the biological sample, thereby generating one or more sequencing reads; (b) aligning the one or more sequencing reads to a reference human genome library and retaining one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. In some embodiments, aligning the one or more sequencing reads is omitted. In some embodiments, predicting further comprises predicting one or more anatomic locations of the cancer of the subject. In some embodiments, the predictive model is further configured to receive the subject's biological sample cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof as an input to predict the cancer. In some embodiments, an area under a receiver operating curve of the predictive model is increased by at least 1%, at least 2%, at least 4%, at least 5%, or at least 10% when the combined decontaminated fungal presence and the decontaminated non-fungal presence is utilized during the correlation.

Another aspect of disclosure described herein comprises a method for training a predictive model based on fungal and non-fungal microbial features to diagnose cancer in a subject, comprising: (a) receiving, from a biological sample of one or more subjects, a fungal presence, a non-fungal microbial presence, and a corresponding health state of the one or more subjects; (b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and (c) training a predictive model with the combined decontaminated fungal presence and the decontaminated non-fungal microbial presence, and the corresponding health state of the one or more subjects. In some embodiments, the non-fungal microbial presence comprises a non-fungal microbial abundance of the biological sample from the one or more subjects. In some embodiments, the fungal presence comprises a fungal abundance of the biological sample from the one or more subjects. In some embodiments, the predictive model is configured to diagnose one or more cancers, one or more subtypes of cancer, one or more of the cancer's anatomic locations, or any combination thereof. In some embodiments, the predictive model is configured to predict a stage of cancer, cancer prognosis, a type of stage I or stage II cancer, a mutation status of one or more cancers, a future immunotherapy response, an optimal therapy, or any combination thereof for one or more subjects. In some embodiments, the predictive model is configured to diagnose one or more stage I or stage II cancers in the one or more subjects. In some embodiments, the predictive model is configured to simultaneously discriminate among one or more cancer types to diagnose a specific cancer type of the subject. In some embodiments, the cancer comprises bone, breast, lung, colon, brain, skin, ovary, pancreas, or any combination thereof type of cancer. In some embodiments, the predictive model is configured to diagnose adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some embodiments, the predictive model is configured to diagnose one or more of the following cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. In some embodiments, removing the contaminating non-fungal microbial features and the contaminating fungal features is completed by in silico decontamination. In some embodiments, removing the contaminating microbial features and the contaminating fungal features is informed by negative experimental controls. In some embodiments, the predictive model comprises a machine learning model, regularized machine learning model, ensemble of machine learning models, or any combination thereof. In some embodiments, the predictive model comprises a random forest, neural network, naïve bayes, support vector machines, linear regression, k-nearest neighbors, k-means, decision tree, logistic regression, gradient boosting, or any combination thereof predictive model. In some embodiments, step (b) improves accuracy of the predictive model by at least 1%, at least 5%, at least 10%, at least 15%, or at least 20%. In some embodiments, step (b) is omitted. In some embodiments, the one or more subjects comprise non-human mammal or human subjects In some embodiments, the biological sample comprises a tissue sample, a liquid biopsy, whole blood biopsy, or any combination thereof samples. In some embodiments, the liquid biopsy comprises whole blood, red blood cells, plasma, white blood cells, saliva, urine, tears, breast milk, or any combination thereof. In some embodiments, the whole blood biopsy comprises plasma, white blood cells, red blood cells, platelets, or any combination thereof. In some embodiments, the fungal presence comprises an abundance of fungal DNA, RNA, methylation, proteins, or any combination thereof. In some embodiments, the non-fungal microbial presence comprises an abundance of non-fungal microbial DNA, RNA, methylation, proteins, or any combination thereof. In some embodiments, detecting the fungal presence and the non-fungal microbial presence in the biological sample comprises: (a) sequencing one or more nucleic acid molecules of the biological sample, thereby generating one or more sequencing reads; (b) aligning the one or more sequencing reads to a reference human genome library and retain one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. In some embodiments, aligning the one or more sequencing reads to a reference human genome library is omitted. In some embodiments, predictive model is configured to predict one or more anatomic locations of a cancer of a subject by providing the trained predictive model an input of a non-fungal microbial presence and a fungal presence of the subject's biological sample. In some embodiments, the predictive model is further trained with cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof. In some embodiments, receiving comprises whole genome sequencing, shotgun sequencing, targeted sequencing, RNA sequencing, methylation sequencing, or any combination thereof sequencing of the fungal and non-fungal microbial presence nucleic acid molecules in the biological sample. In some embodiments, the health state of the one or more subjects comprises a non-cancerous health state or cancerous health state. In some embodiments, the non-cancerous health state comprises a non-cancerous disease health state or a non-diseased health state.

Another aspect of the disclosure described herein comprises a method for training a predictive model based on fungal and non-fungal microbial features to predict cancer in a subject, comprising: (a) receiving a fungal presence, a non-fungal microbial presence, and a health state of one or more subjects from a database; (b) removing contaminating fungal features of the fungal presence and contaminating non-fungal microbial features the non-fungal microbial presence while retaining decontaminated fungal features and decontaminated non-fungal microbial features, thereby producing a combined decontaminated fungal presence and a decontaminated non-fungal microbial presence; and (c) training a predictive model configured to predict cancer in a subject with the combined decontaminated fungal presence and decontaminated non-fungal microbial presence, and the corresponding health state of the one or more subjects. In some embodiments, the non-fungal microbial presence comprises a non-fungal microbial abundance of the biological sample from the one or more subjects. In some embodiments, the fungal presence comprises a fungal abundance of the biological sample from the one or more subjects. In some embodiments, the predictive model is configured to diagnose one or more cancers, one or more subtypes of cancer, one or more of its anatomic locations, or any combination thereof. In some embodiments, the predictive model is configured to predict a stage of cancer, cancer prognosis, a type of stage I or stage II cancer, a mutation status of one or more cancers, a future immunotherapy response, an optimal therapy, or any combination thereof for one or more subjects. In some embodiments, the predictive model is configured to diagnose one or more stage I or stage II cancers in the one or more subjects. In some embodiments, the predictive model is configured to simultaneously discriminate among one or more cancer types to diagnose a specific cancer type of the subject. In some embodiments, the associated type of cancer comprises bone, breast, lung, colon, brain, skin, ovary, pancreas, or any combination thereof type of cancer. In some embodiments, the predictive model is configured to diagnose adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some embodiments, the predictive model is configured to diagnose one or more of the following cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. In some embodiments, removing the contaminated microbial features and the contaminated fungal features is completed by in silico decontamination. In some embodiments, removing the contaminated microbial features and the contaminated fungal features is informed by experimental controls. In some embodiments, the predictive model comprises a machine learning model, regularized machine learning model, ensemble of machine learning models, or any combination thereof. In some embodiments, the predictive model comprises a random forest, neural network, naïve bayes, support vector machines, linear regression, k-nearest neighbors, k-means, decision tree, logistic regression, gradient boosting, or any combination thereof predictive model. In some embodiments, step (b) improves accuracy of the predictive model by at least 1%, at least 5%, at least 10%, at least 15%, or at least 20%. In some embodiments, step (b) is omitted. In some embodiments, the one or more subjects comprise non-human mammal or human subjects. In some embodiments, the biological sample comprises a tissue sample, a liquid biopsy, whole blood biopsy, or any combination thereof samples. In some embodiments, the liquid biopsy comprises whole blood, red blood cells, plasma, white blood cells, saliva, urine, tears, breast milk, or any combination thereof. In some embodiments, the whole blood biopsy comprises plasma, white blood cells, red blood cells, platelets, or any combination thereof. In some embodiments, the fungal presence comprises an abundance of fungal DNA, RNA, methylation, proteins, or any combination thereof. In some embodiments, the non-fungal microbial presence comprises an abundance of non-fungal microbial DNA, RNA, methylation, proteins, or any combination thereof. In some embodiments, detecting the fungal presence and the non-fungal microbial presence in the biological sample comprises: (a) sequencing one or more nucleic acid molecules of the biological sample, thereby generating one or more sequencing reads; (b) aligning the one or more sequencing reads to a reference human genome library and retain one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. In some embodiments, aligning the one or more sequencing reads to reference human genome library is omitted. In some embodiments, predictive model is configured to predict a bodily location of a cancer of a subject by providing the trained predictive model an input of a non-fungal microbial presence and a fungal presence of the subject's biological sample. In some embodiments, the predictive model is further trained with cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof. In some embodiments, detecting comprises whole genome sequencing, shotgun sequencing, targeted sequencing, RNA sequencing, methylation sequencing, or any combination thereof sequencing of the fungal and non-fungal microbial presence nucleic acid molecules. In some embodiments, the database comprises The Cancer Genome Atlas database (TCGA), the International Cancer Genome Consortium (ICGC) database, the Pan-Cancer Atlas of Whole Genomes (PCAWG) database, the Therapeutically Applicable Research to Generate Effective Treatments (TARGET) database, the Clinical Proteomic Tumor Analysis Consortium (CPTAC) database, the Hartwig Medical Foundation (HMF) metastasis database, the Tracking Non-Small-Cell Lung Cancer Evolution through Therapy (TRACERx) database, the 100,000 Genomes Project, or any combination thereof. In some embodiments, the health state of the one or more subjects comprises a non-cancerous health state or cancerous health state. In some embodiments, the non-cancerous health state comprises a non-cancerous disease health state or a non-diseased health state.

Another aspect of the disclosure described herein comprises a method of treating cancer of a subject based on a combined non-fungal microbial and fungal presence of a biological sample of the subject, comprising: (a) detecting a fungal presence and a non-fungal microbial presence in a biological sample from a subject; (b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and (c) administering a therapeutic to treat a cancer of the subject determined by at least a correlation between the combined decontaminated fungal presence and the decontaminated non-fungal microbial presence of the subject to a known combined fungal presence and non-fungal microbial presence of subjects with cancer treated with the therapeutic. In some embodiments, the non-fungal microbial presence comprises a non-fungal microbial abundance of the biological sample from the one or more subjects. In some embodiments, the fungal presence comprises a fungal abundance of the biological sample from the one or more subjects. In some embodiments, the cancer of the comprises one or more cancers, one or more subtypes of cancer, or any combination thereof. In some embodiments, the cancer comprises a stage I or stage II cancer. In some embodiments, the cancer comprises bone, breast, lung, colon, brain, skin, ovary, pancreas, or any combination thereof type of cancer. In some embodiments, the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some embodiments, the cancer comprises a cancer type outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. In some embodiments, removing the contaminating non-fungal microbial features and the contaminating fungal features is completed by in silico decontamination. In some embodiments, removing the contaminating non-fungal microbial features and the contaminating fungal features is informed by experimental controls. In some embodiments, the correlation is determined by a predictive model, wherein the predictive model comprises a machine learning model, regularized machine learning model, ensemble of machine learning models, or any combination thereof. In some embodiments, the predictive model comprises a random forest, neural network, naïve bayes, support vector machines, linear regression, k-nearest neighbors, k-means, decision tree, logistic regression, gradient boosting, or any combination thereof predictive model. In some embodiments, step (b) improves accuracy of the predictive model by at least 1%, at least 5%, at least 10%, at least 15%, or at least 20%. In some embodiments, step (b) is omitted. In some embodiments, the subject comprises a non-human mammal or human subject. In some embodiments, the biological sample comprises a tissue sample, a liquid biopsy, whole blood biopsy, or any combination thereof samples In some embodiments, the liquid biopsy comprises whole blood, red blood cells, plasma, white blood cells, saliva, urine, tears, breast milk, or any combination thereof. In some embodiments, the whole blood biopsy comprises plasma, white blood cells, red blood cells, platelets, or any combination thereof. In some embodiments, the fungal presence comprises an abundance of fungal DNA, RNA, methylation, proteins, or any combination thereof. In some embodiments, the non-fungal microbial presence comprises an abundance of non-fungal microbial DNA, RNA, methylation, proteins, or any combination thereof. In some embodiments, detecting the fungal presence and the non-fungal microbial presence in the biological sample comprises: (a) sequencing one or more nucleic acid molecules of the biological sample, thereby generating one or more sequencing reads; (b) aligning the one or more sequencing reads to a reference human genome library and retain one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. In some embodiments, the predictive model is trained with one or more subject's biologic sample decontaminated fungal presence, decontaminated non-fungal microbial presence cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof, a corresponding subject's cancer, and treatment provided to treat the subject's cancer. In some embodiments, detecting comprises whole genome sequencing, shotgun sequencing, targeted sequencing, RNA sequencing, methylation sequencing, or any combination thereof sequencing of the fungal and non-fungal microbial presence nucleic acid molecules. In some embodiments, the treatment repurposes an existing medication, which may or may not have been originally approved for targeting cancer. In some embodiments, the treatment comprises a small molecule, a biologic, a probiotic, a virus, a bacteriophage, immunotherapy, broad spectrum antibiotic, or any combination thereof. In some embodiments, the probiotic comprises an engineered bacterium strain or ensemble of engineered bacteria. In some embodiments, the treatment comprises an adjuvant given in combination with a primary treatment against the cancer to improve the efficacy of the primary treatment. In some embodiments, the treatment comprises adoptive cell transfer to target microbial antigens associated with the cancer or cancer microenvironment. In some embodiments, the treatment comprises a cancer vaccine that exploits microbial antigens associated with the cancer or cancer microenvironment. In some embodiments, the treatment comprises a monoclonal antibody against microbial antigens associated with the cancer or cancer microenvironment. In some embodiments, the treatment comprises an antibody-drug conjugate designed to at least partially target microbial antigens associated with the cancer or cancer microenvironment. In some embodiments, the treatment comprises a multi-valent antibody, antibody fragment, or antibody derivative thereof designed to at least partially target one or more microbial antigens associated with the cancer or cancer microenvironment. In some embodiments, the treatment comprises a targeted antibiotic against a particular kind of microbe or class of functionally or biologically similar microbes. In some embodiments, two or more of the following treatment types are combined such that at least one type exploits the cancer microbial presence or abundance to enhance overall therapeutic efficacy: small molecules, biologics, engineered host-derived cell types, probiotics, engineered bacteria, natural-but-selective viruses, engineered viruses, and bacteriophages.

Another aspect of the disclosure described herein comprises a computer-implemented method for utilizing a predictive model to predict cancer of a subject from a combined fungal and non-fungal microbial presence of a biological sample, comprising: (a) detecting a fungal presence and a non-fungal microbial presence in a biological sample from a subject; (b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and (c) predicting, using a computer that implements the predictive model, a cancer of the subject by correlating the combined decontaminated fungal presence and the decontaminated non-fungal microbial presence of the subject to a known combined fungal presence and non-fungal microbial presence for one or more cancers. In some embodiments, detecting comprises whole genome sequencing, shotgun sequencing, targeted sequencing, RNA sequencing, methylation sequencing, or any combination thereof. In some embodiments, the non-fungal microbial presence comprises bacteria, viruses, archaea, protists, or any combination thereof. In some embodiments, the non-fungal microbial presence comprises a non-fungal microbial abundance of the biological sample from the subject. In some embodiments, the fungal presence comprises a fungal abundance of the biological sample from the subject. In some embodiments, predicting the cancer further comprises predicting one or more cancers, one or more subtypes of cancer, the anatomic locations of one or more cancers, or any combination thereof in the subject. In some embodiments, predicting the cancer comprises predicting a stage of the cancer, cancer prognosis, a mutation status of the cancer, a future immunotherapy response of the cancer, an optimal therapy to treat the cancer, or any combination thereof for one or more subjects. In some embodiments, the cancer comprises a stage I or stage II cancer. In some embodiments, predicting the cancer comprises predicting a cancer type among one or more cancer types. In some embodiments, predicting the cancer comprises simultaneously discriminating among one or more cancer types to diagnose a specific cancer type of the subject. In some embodiments, the cancer comprises bone, breast, lung, colon, brain, skin, ovary, pancreas, or any combination thereof type of cancer. In some embodiments, the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some embodiments, the cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. In some embodiments, removing the contaminating non-fungal microbial features and the contaminating fungal features is completed by in silico decontamination. In some embodiments, removing the contaminating non-fungal microbial features and the contaminating fungal features is informed by experimental contamination controls. In some embodiments, the predictive model comprises a machine learning model, regularized machine learning model, ensemble of machine learning models, or any combination thereof. In some embodiments, the predictive model comprises a random forest, neural network, naïve bayes, support vector machines, linear regression, k-nearest neighbors, k-means, decision tree, logistic regression, gradient boosting, or any combination thereof predictive model. In some embodiments, step (b) improves accuracy of the predictive model by at least 1%, at least 5%, at least 10%, at least 15%, or at least 20%. In some embodiments, step (b) is omitted. In some embodiments, the subject comprises a non-human mammal or a human subject. In some embodiments, the biological sample comprises a tissue sample, a liquid biopsy, whole blood biopsy, or any combination thereof samples. In some embodiments, the liquid biopsy comprises whole blood, red blood cells, plasma, white blood cells, saliva, urine, tears, breast milk, or any combination thereof. In some embodiments, the whole blood biopsy comprises plasma, white blood cells, red blood cells, platelets, or any combination thereof. In some embodiments, the fungal presence comprises an abundance of fungal DNA, RNA, methylation, proteins, or any combination thereof. In some embodiments, the non-fungal microbial presence comprises an abundance of non-fungal microbial DNA, RNA, methylation, proteins, or any combination thereof. In some embodiments, detecting the fungal presence and the non-fungal microbial presence in the biological sample comprises: (a) sequencing one or more nucleic acid molecules of the biological sample, thereby generating one or more sequencing reads; (b) aligning the one or more sequencing reads to a reference human genome library and retaining one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. In some embodiments, aligning the one or more sequencing reads is omitted. In some embodiments, predicting further comprises predicting one or more anatomic locations of the cancer of the subject. In some embodiments, the predictive model is further configured to receive the subject's biological sample cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof as an input to predict the cancer. In some embodiments, detecting comprises whole genome sequencing, shotgun sequencing, targeted sequencing, RNA sequencing, methylation sequencing, or any combination thereof the one or more nucleic acid molecules of the biological sample. In some embodiments, an area under a receiver operating curve of the predictive model for predicting the cancer of the subject is increased by at least 1%, at least 2%, at least 4%, at least 5%, or at least 10% when the combined decontaminated fungal presence and the decontaminated non-fungal presence is utilized during the correlation.

Another aspect of the disclosure described herein comprises a computer system configured to predict cancer of a subject from a combined fungal and non-fungal microbial presence of a biological sample, comprising: (a) one or more processors; and (b) a non-transient computer readable storage medium including software, wherein the software comprises executable instructions that, as a result of the execution, cause the one or more processors of the computer system to: (i) detect a fungal presence and a non-fungal microbial presence in a biological sample from a subject; (ii) remove contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and (iii) predict a cancer of the subject by correlating the combined decontaminated fungal presence and the decontaminated non-fungal microbial presence of the subject to a known combined fungal presence and non-fungal microbial presence for one or more cancers. In some embodiments, detecting comprises whole genome sequencing, shotgun sequencing, targeted sequencing, RNA sequencing, methylation sequencing, or any combination thereof. In some embodiments, the non-fungal microbial presence comprises bacteria, viruses, archaea, protists, or any combination thereof. In some embodiments, the non-fungal microbial presence comprises a non-fungal microbial abundance of the biological sample from the subject. In some embodiments, the fungal presence comprises a fungal abundance of the biological sample from the subject. In some embodiments, predicting the cancer further comprises predicting one or more cancers, one or more subtypes of cancer, the anatomic locations of one or more cancers, or any combination thereof in the subject. In some embodiments, predicting the cancer comprises predicting a stage of the cancer, cancer prognosis, a mutation status of the cancer, a future immunotherapy response of the cancer, an optimal therapy to treat the cancer, or any combination thereof for one or more subjects. In some embodiments, the cancer comprises a stage I or stage II cancer. In some embodiments, predicting the cancer comprises predicting a cancer type among one or more cancer types. In some embodiments, predicting the cancer comprises simultaneously discriminating among one or more cancer types to diagnose a specific cancer type of the subject. In some embodiments, the cancer comprises bone, breast, lung, colon, brain, skin, ovary, pancreas, or any combination thereof type of cancer. In some embodiments, the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some embodiments, the cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. In some embodiments, removing the contaminating non-fungal microbial features and the contaminating fungal features is completed by in silico decontamination. In some embodiments, removing the contaminating non-fungal microbial features and the contaminating fungal features is informed by experimental contamination controls. In some embodiments, the predictive model comprises a machine learning model, regularized machine learning model, ensemble of machine learning models, or any combination thereof. In some embodiments, the predictive model comprises a random forest, neural network, naïve bayes, support vector machines, linear regression, k-nearest neighbors, k-means, decision tree, logistic regression, gradient boosting, or any combination thereof predictive model. In some embodiments, step (b) improves accuracy of the predictive model by at least 1%, at least 5%, at least 10%, at least 15%, or at least 20%. In some embodiments, step (b) is omitted. In some embodiments, the subject comprises a non-human mammal or a human subject. In some embodiments, the biological sample comprises a tissue sample, a liquid biopsy, whole blood biopsy, or any combination thereof samples. In some embodiments, the liquid biopsy comprises whole blood, red blood cells, plasma, white blood cells, saliva, urine, tears, breast milk, or any combination thereof. In some embodiments, the whole blood biopsy comprises plasma, white blood cells, red blood cells, platelets, or any combination thereof. In some embodiments, the fungal presence comprises an abundance of fungal DNA, RNA, methylation, proteins, or any combination thereof. In some embodiments, the non-fungal microbial presence comprises an abundance of non-fungal microbial DNA, RNA, methylation, proteins, or any combination thereof. In some embodiments, detecting the fungal presence and the non-fungal microbial presence in the biological sample comprises: (a) sequencing one or more nucleic acid molecules of the biological sample, thereby generating one or more sequencing reads; (b) aligning the one or more sequencing reads to a reference human genome library and retaining one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. In some embodiments, aligning the one or more sequencing reads to a reference human genome library is omitted. In some embodiments, predicting further comprises predicting one or more anatomic locations of the cancer of the subject. In some embodiments, the predictive model is further configured to receive the subject's biological sample cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof, as an input to predict the cancer. In some embodiments, detecting comprises whole genome sequencing, shotgun sequencing, targeted sequencing, RNA sequencing, methylation sequencing, or any combination thereof the one or more nucleic acid molecules of the biological sample. In some embodiments, an area under a receiver operating curve of the predictive model for predicting the cancer of the subject is increased by at least 1%, at least 2%, at least 4%, at least 5%, or at least 10% when the combined decontaminated fungal presence and the decontaminated non-fungal presence is utilized during the correlation.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a workflow diagram of a method of detecting cancer of a subject with a combined fungal and non-fungal microbial presence, as described in embodiments herein.

FIGS. 2A-2B show workflow diagrams of methods to train a predictive model to detect a subject's cancer from a fungal and non-fungal microbial presence, as described in embodiments herein.

FIG. 3 shows a workflow diagram of a method of administering a therapeutic to treat a cancer of a subject based at least on the subject's fungal and non-fungal microbial presence, as described in embodiments herein.

FIG. 4 shows a workflow diagram of a computer-implemented method of predicting a cancer of a subject by the subject's fungal and non-fungal microbial presence in a biological sample, as described in embodiments herein.

FIGS. 5A-5C show beta diversity analyses of fungal abundances derived from treatment-naïve, whole genome sequenced primary tumors within single sequencing centers, suggesting cancer-type specific mycobiomes that are more similar to their normal adjacent tissue (NAT) than other cancer types, as described in embodiments herein. Cancer type naming abbreviations are noted in Table 1.

FIGS. 6A-6E show graphs of alpha diversity of fungal abundances derived from treatment-naïve, whole genome sequenced primary tumors within single sequencing centers, suggesting cancer-type specific mycobiomes, as described in embodiments herein. Cancer type naming abbreviations are noted in Table 1.

FIG. 7 shows a graph of decontamination results based on 325 plate-center batches in TCGA using analyte concentrations from 12,878 samples, as described in embodiments herein.

FIGS. 8A-8C show graphs of data batch effects in The Cancer Genome Atlas (TCGA) mycobiome data, potentially due to differences in read depths between whole genome sequenced samples and RNA sequenced samples-differences that are mitigated using Voom-SNM, as described in embodiments herein.

FIGS. 9A-9D show graphs quantitatively representing the data improvement following batch effect correction by a concomitant reduction in technical effects; predictive modeling performances on pan-cancer, TCGA batch-corrected fungal data that are consistently higher in biological samples than scrambled or shuffled data counterparts; and correlated performances when splitting the data into halves, performing batch correction on each half separately, training predictive models on each half independently, and testing the predictive model on the counterpart half of the batch-corrected data. Cancer type naming abbreviations are noted in Table 1.

FIG. 10 shows a workflow diagram for processing and detecting a fungal and non-fungal microbial presence of a biological sample, as described in embodiments herein.

FIGS. 11A-11B show data for an example validation cohort and decontamination of blood-derived plasma mycobiome.

FIG. 12 shows a system configured to implement the methods of the disclosure, as described in embodiments herein.

FIG. 13 shows a graph representing percentage of fungal or non-fungal bacterial reads in TCGA primary tumors versus total reads, and their correlation, as described in embodiments herein. Cancer type naming abbreviations are noted in Table 1.

FIGS. 14A-14H show graphs of machine learning performances that reveal cancer type-specific tumor and blood mycobiomes that are statistically significantly better than scrambled or shuffled controls, using samples from the TCGA database, as well as synergistic performance enhancements when combining fungal and non-fungal microbial features, as described in embodiments herein. Cancer type naming abbreviations are noted in Table 1. “WIS” and “Weizmann” both denote independent data from the Weizmann Institute.

FIGS. 15A-15D show graphs of receiver operating characteristic curves, precision recall curves, and corresponding area under the curves thereof for clinical predictive performance of plasma-derived fungal and non-fungal microbial abundances, with synergy when combining them, in as early as stage I cancer, as well as a subset of 20 fungal species that provide as much discriminative performance as more than 200 species, as described in embodiments herein. Table 3 lists the 20 fungal species shown in this analysis.

FIGS. 16A-16D show graphs of the distribution of fungal nucleic acids across cancer types and sample types, inclusive of primary tumors and blood among others, as described in embodiments herein. Cancer type naming abbreviations are noted in Table 1.

FIGS. 17A-17F show graphs of data distribution of pan-microbial and non-fungal bacterial nucleic acids across TCGA cancer types and the pan-cancer comparison of genome-normalized fungal versus non-fungal bacterial proportions, as described in embodiments herein. Cancer type naming abbreviations are noted in Table 1.

FIGS. 18A-18E show graphs of the comparison of pan-cancer fungal and non-fungal bacterial read proportions in TCGA cancer data, and their correlations, as described in embodiments herein. Cancer type naming abbreviations are noted in Table 1.

FIGS. 19A-19B shows graphs of fungal genera or species overlap between Weizmann (WIS) and TCGA cancer cohorts on a per-cancer type basis, as described in embodiments herein. The intersected is bounded by the taxonomic database intersection used in the two cohorts.

FIGS. 20A-20P show graphs of machine learning classifier performance TCGA samples using fungal data to distinguish one cancer type versus all others, within single sequencing centers to bypass the need to batch correct the data; the superior performance of whole genome sequenced samples over RNA sequenced samples, potentially due to differences in sequencing depth; the differences in minority class sizes that may explain differences in machine learning performances between cancer types; and the similarities in performances when using subsets of fungal species found in independent datasets (e.g., the Weizmann) or taxonomic calling algorithms (e.g., EukDetect); as described in embodiments herein. Cancer type naming abbreviations are noted in Table 1.

FIGS. 21A-21G shows graphs of machine learning classifier performance trained on TCGA subsets of raw fungal count data summarized to various taxa levels in single sequencing centers to bypass batch correction to distinguish one cancer type versus all others, as described in embodiments herein. Cancer type naming abbreviations are noted in Table 1.

FIGS. 22A-22H show graphs evaluating biological samples versus scrambled or shuffled negative data controls for machine learning on TCGA raw data in single sequencing centers to bypass batch correction, as well machine learning performance on independent stratified halves that are cross-tested on each other, as described in embodiments herein.

FIGS. 23A-23G show representative differential abundance volcano plots of one cancer type versus all other using intratumoral decontaminated fungi in TCGA, as described in embodiments herein.

FIGS. 24A-24E show graphs evaluating WIS-associated features—fungal and/or non-fungal bacterial abundances—in TCGA and in the WIS-cohort for machine learning discriminatory performance, as described in some embodiments herein. GBM: glioblastoma; PDA: pancreatic ductal adenocarcinoma; LC, lung cancer; SARC, sarcoma; OV, ovarian cancer; SKCM, melanoma; BRCA, breast cancer.

FIGS. 25A-25K show differential abundance volcano plots of stage I versus stage IV tumors using intratumoral decontaminated fungi in TCGA, as described in some embodiments herein.

FIGS. 26A-26I show graphs of TCGA and WIS trained machine learning performance when differentiating between stage I and stage IV tumors and tumors versus normal tissue adjacent to the tumor (NAT) using fungal and/or non-fungal bacterial abundances. Cancer type naming abbreviations are noted in Table 1 except for LC, which is lung cancer.

FIGS. 27A-27E show graphs of representative differential abundance volcano plots of one cancer type versus all others using blood-derived decontaminated fungi in TCGA, as described in embodiments herein.

FIGS. 28A-28D show graphs of the performance of machine learning models trained on TCGA subsets (single sequencing centers to bypass batch correction) of raw fungal count data to distinguish blood samples from one cancer type versus all others, as described in embodiments herein. Cancer type naming abbreviations are noted in Table 1.

FIGS. 29A-29E show graphs of the performance of machine learning models trained on TCGA subsets (single sequencing centers to bypass batch correction) of raw fungal count data summarized to various taxa levels to distinguish blood samples from one cancer type versus all others, as described in embodiments herein. Cancer type naming abbreviations are noted in Table 1.

FIGS. 30A-30G show graphs evaluating biological samples negative scrambled and shuffled data controls for machine learning models trained on TCGA blood raw data, as well as performances when utilizing WIS-overlapping fungal features, as described in embodiments herein.

FIGS. 31A-31C show graphs of biological samples and negative scrambled and shuffled data controls for machine learning models trained on TCGA pan-cancer batch-corrected blood sample, as well as one cancer type versus all other machine learning performance when restricting the analyses to patients with stage I-II tumors, as described in embodiments herein. Cancer type naming abbreviations are noted in Table 1.

FIGS. 32A-32G show graphs of similarities in machine learning performance when utilizing various machine learning model types for cancer type discrimination in TCGA using batch-corrected and raw decontaminated data, inclusive of data summarized at various taxonomic levels, as described in embodiments herein. Cancer type naming abbreviations are noted in Table 1. GBM, gradient boosting machines; RF, random forests; CV, cross-validation.

FIGS. 33A-33G show graphs of similarities in performances when using different sampling strategies during machine learning training for cancer type discrimination in TCGA using batch-corrected and raw decontaminated data, inclusive of data summarized at various taxonomic levels, as described in embodiments herein. Cancer type naming abbreviations are noted in Table 1. CV, cross validation.

FIGS. 34A-34F show graphs of machine learning performances in the Hopkins dataset when discriminating cancer versus healthy samples when using plasma-derived mycobiomes; the performance of biological samples versus negative shuffled and scrambled data controls; and log-ratios of the fungi originally identified in the TCGA mycotypes testing for significant cancer type variation, as described in embodiments herein.

FIGS. 35A-35H show graphs of machine learning model performance in one cancer type versus all others, cancer versus healthy samples, the performance stability of the latter across various cancer stages, the identification of a subset of 20 fungal species that provide discriminatory performance better than >200 total fungal species, the utility of those 20 fungal species in two independent datasets (TCGA, University of California San Diego (UCSD)), and the replication of similar fungal-driven machine learning performances in another independent cohort (UCSD), as described in embodiments herein. Cancer type naming abbreviations are noted in Table 1.

FIGS. 36A-36D show graphs of additional machine learning and control analyses of decontaminated fungal abundances in UCSD cohort plasma samples comparing between cancer types, cancer versus healthy samples, and predicting immunotherapy responders, as described in embodiments herein.

FIG. 37 shows a table of identified contaminates determined from analysis, as described in embodiments herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

Fungi are understudied but important commensals and/or opportunistic pathogens that shape host immunity and infect immunocompromised e.g., cancer patients. Fungi have been found in individual tumor types, and contribute to carcinogenesis in a few cancer types, but their presence, identify, location, and effects in most cancer types are unknown.

Cancer-microbe associations have been explored for centuries but cancer-associated fungi have rarely been examined for their cancer diagnostic capabilities. Disclosed herein, in some embodiments are methods and systems configured to detect fungal presence and features of a subject and/or subjects' biologic sample(s) to predict a disease of the subject and/or subjects. In some instances, the disease may comprise cancer. In some cases, the methods and systems described herein may train a predictive model, where the trained predictive model may diagnose or predict cancer of a subject or subjects when provided, as an input, a fungal presence, a non-fungal microbial presence, or a combination thereof. In some instances, the methods and systems described herein may comprise a method of predicting a cancer of a subject with a combined fungal and non-fungal microbial presence of the subject's biological sample. By combining the fungal and non-fungal microbial presence an unexpected improvement in predictive performance of the predictive model may be achieved and/or realized. Even though fungi represent a fraction (e.g., 0.002% of total reads detected in a biological sample), combining a biological sample's fungal presence with non-fungal microbial presence improves predictive accuracy of the non-fungal microbial presence when predicting a cancer of a subject.

### Methods

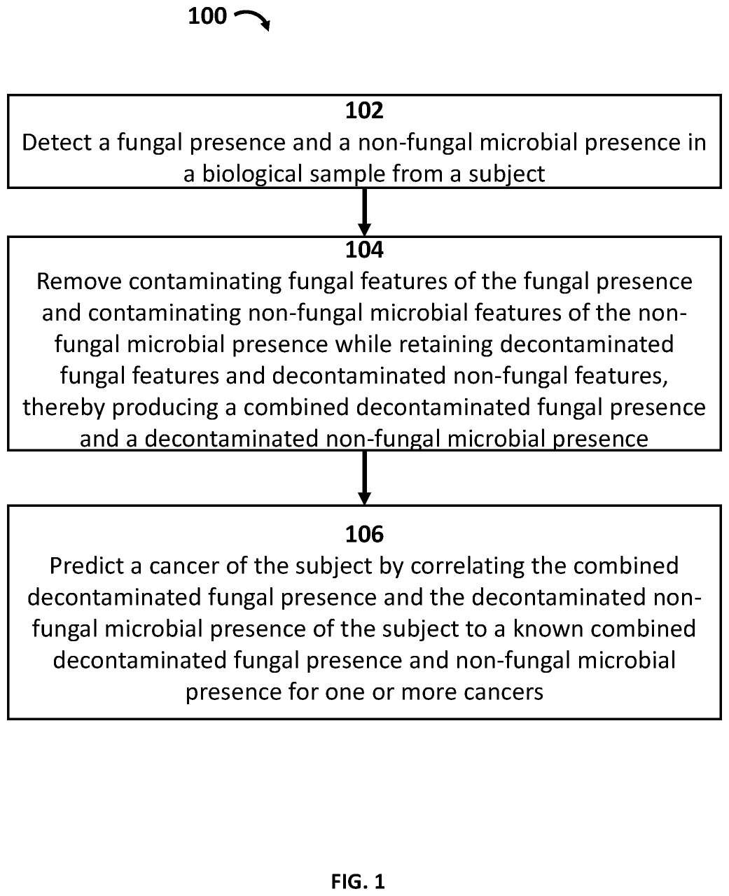

Aspects of the disclosure provided herein describe a method of predicting cancer of a subject from a combined fungal and non-fungal microbial presence of a biological sample 100, as shown in FIG. 1. In some cases, the method may comprise: (a) detecting a fungal presence and a non-fungal presence in a biological sample from a subject 102; (b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence 104; and (c) predicting a cancer of the subject by correlating the combined decontaminated fungal presence and the decontaminated non-fungal microbial presence of the subject to a known combined fungal presence and non-fungal microbial presence for one or more cancers. In some cases, the subject may comprise a non-human mammal or a human subject 106. In some instances, the biological sample may comprise a tissue sample, a liquid biopsy, whole blood biopsy, or any combination thereof samples. In some cases, the liquid biopsy may comprise whole blood, red blood cells, plasma, white blood cells, saliva, urine, tears, breast milk, or any combination thereof. In some instances, the whole blood biopsy may comprise plasma, white blood cells, red blood cells, platelets, or any combination thereof.

In some cases, detecting may comprise whole genome sequencing, shotgun sequencing, targeted sequencing, RNA sequencing, methylation sequencing, or any combination thereof. In some instances, detecting the fungal presence and the non-fungal microbial presence in the biological sample may comprise: (a) sequencing one or more nucleic acid molecules of the biological sample, thereby generating one or more sequencing reads; (b) aligning the one or more sequencing reads to a reference human genome library and retaining one or more non-human sequencing reads that do not align to the reference human genome library; and (c) mapping the one or more non-human sequencing reads to a fungal and a non-fungal microbial reference genome library thereby generating a fungal presence and a non-fungal microbial presence of the biological sample. In some cases, aligning the one or more sequencing reads to a reference human genome library may be omitted from detecting.

In some cases, mapping the one or more non-human sequencing reads to a fungal and non-fungal microbial reference genome library may comprise mapping to a functional genome database to generate one or more functional genomic features. In some instances, the functional genome database may comprise the Kyoto Encyclopedia of Genes and Genomes (KEGG). The one or more functional genomic features may comprise one or more metabolic features associated with one or more non-human sequencing reads. In some cases, the one or more metabolic features may comprise functional units of gene sets in metabolic pathways, functional units of gene sets that characterize phenotypic features, functional units of successive reaction steps in metabolic pathways, or any combination thereof. For example, as a result of mapping the one or more non-human sequencing reads to the KEGG database's one or more metabolic pathways, a presence and/or abundance of enzymes and/or their reaction products based on the one or more non-human sequencing reads, or any combination thereof, may be generated. In some cases, the one or more pathways may be utilized as features in addition to or in place of the one or more fungal and non-fungal microbial presence and abundance features to train a predictive model, described elsewhere herein.

In some instances, the non-fungal microbial presence may comprise bacteria, viruses, archaea, protists, or any combination thereof. In some cases, the non-fungal microbial presence may comprise a non-fungal microbial abundance of the biological sample from the subject. In some cases, the fungal presence may comprise a fungal abundance of the biological sample from the subject. In some cases, the fungal presence may comprise an abundance of fungal DNA, RNA, methylation, proteins, or any combination thereof. In some instances, the non-fungal microbial presence may comprise an abundance of non-fungal microbial DNA, RNA, methylation, proteins, or any combination thereof.

In some instances, predicting the cancer may further comprise predicting one or more cancers, one or more subtypes of cancer, the anatomic location of one or more cancers, or any combination thereof in the subject. In some cases, predicting the cancer may comprise predicting a stage of the cancer, cancer prognosis, a mutation status of the cancer, a future immunotherapy response of the cancer, an optimal therapy to treat the cancer, or any combination thereof for one or more subjects. In some instances, predicting the cancer may comprise predicting a cancer type among one or more cancer types. In some cases, predicting may further comprise predicting one or more anatomical locations of the cancer of the subject.

In some cases, the cancer may comprise a stage I or stage II cancer. In some instances, the cancer may comprise bone, breast, lung, colon, brain, skin, ovary, pancreas, or any combination thereof type of cancer. In some instances, the cancer may comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some cases, the cancer may comprises one or more cancer types outside the intestine comprising: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some cases, removing the contaminating non-fungal microbial features and the contaminating fungal features may be completed by in silico decontamination. In some instances, removing the contaminating non-fungal microbial features and the contaminating fungal features may be informed by experimental contamination controls, e.g., measuring fungal and non-fungal abundances in negative control samples and removing identified contaminants from the fungal and/or non-fungal microbial presence detected from a biological sample.

In some instances, predicting may be conducted with a predictive model, where the predictive model may comprise a machine learning model, regularized machine learning model, ensemble of machine learning models, or any combination thereof predictive models. In some cases, removing contaminating fungal and non-fungal microbial features may improve performance of the predictive model by at least 1%, at least 5%, at least 10%, at least 15% or at least 20% improvement. In some cases, removing contaminating fungal and non-fungal microbial features may be omitted from the method. In some cases, the predictive model may be further configured to receive the subject's biological sample cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof as an input to predict the cancer of the subject. In some cases, an area under a receiver operating characteristic curve of the predictive model may increase by at least 1%, at least 2%, at least 4% at least 5%, or at least 10% when the combined decontaminated fungal presence and decontaminated non-fungal presence are utilized during correlation.

In some cases, the predictive model may comprise a random forest, neural network, naïve bayes, support vector machines, linear regression, k-nearest neighbors, k-means, decision tree, logistic regression, gradient boosting, or any combination thereof predictive models.

Training a Predictive Model from a Biological Sample

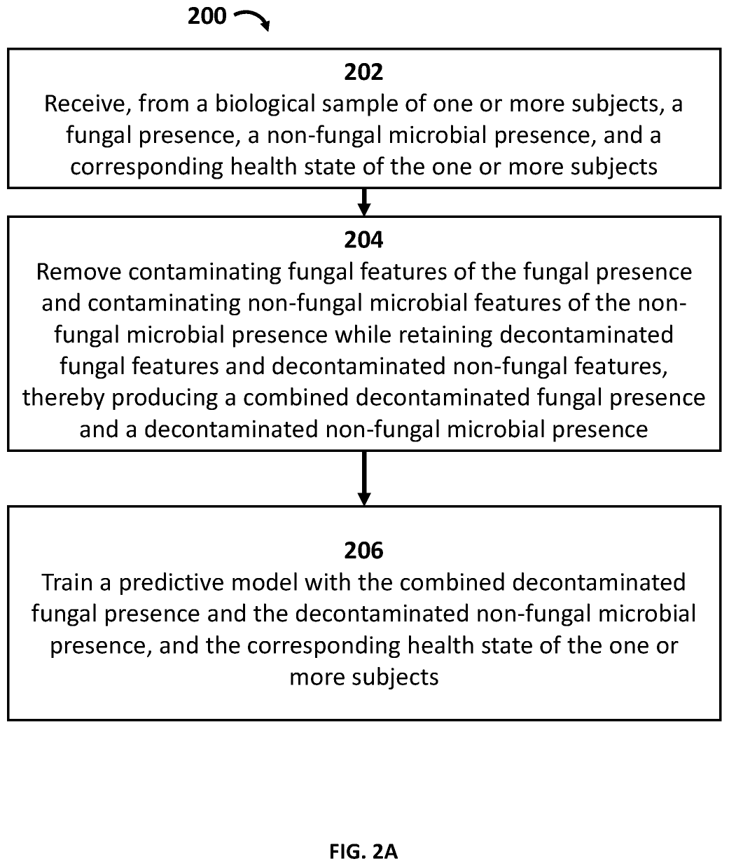

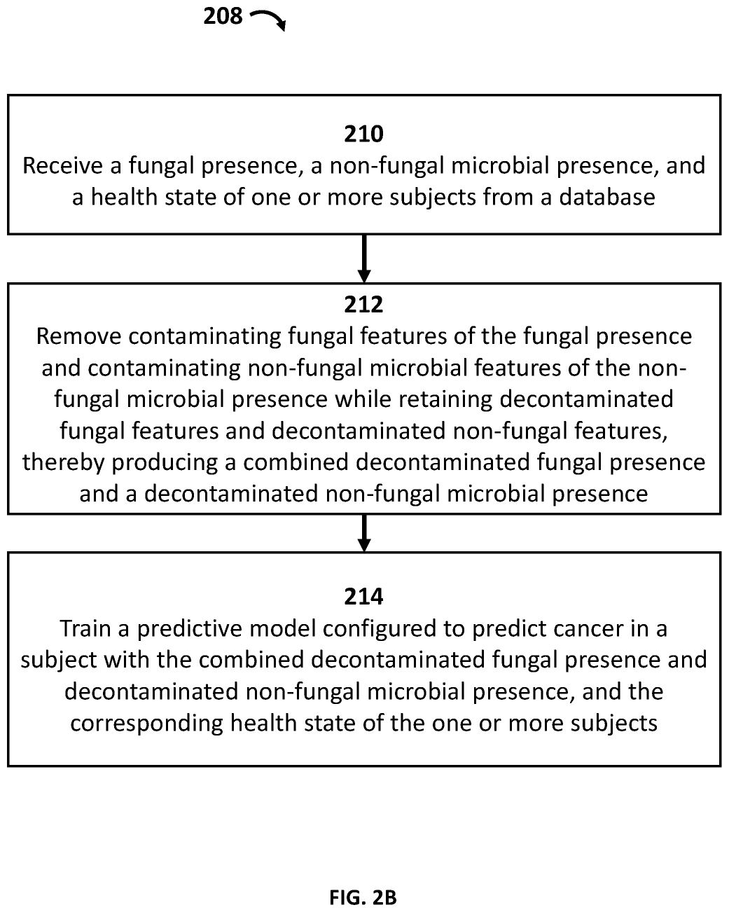

Another aspect of the disclosure may describe a method for training a predictive model based on fungal and non-fungal microbial features to diagnose cancer in a subject 200, as seen in FIG. 2A. In some cases, the method may comprise: (a) receiving, from a biological sample of one or more subjects, a fungal presence, a non-fungal microbial presence, and a corresponding health state of the one or more subjects 202; (b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence 204; (c) training a predictive model with the combined decontaminated fungal presence and the decontaminated non-fungal microbial presence, and the corresponding health state of the one or more subjects. In some cases, the non-fungal microbial presence may comprise a non-fungal microbial abundance of the biological sample from the one or more subjects. In some instances, the fungal presence may comprise a fungal abundance of the biological sample from the one or more subjects. In some cases, the fungal presence may comprise an abundance of fungal DNA, RNA, methylation, proteins, or any combination thereof. In some cases, the non-fungal microbial presence may comprise an abundance of non-fungal microbial DNA, RNA, methylation proteins, or any combination thereof.

In some cases, the one or more subjects may comprise non-human mammal or human subjects. In some cases, the biological sample may comprise a tissue sample, a liquid biopsy, whole blood biopsy, or any combination thereof samples. In some instances, the liquid biopsy may comprise whole blood, red blood cells, plasma, white blood cells, saliva, urine, tears, breast milk, or any combination thereof. In some cases, the whole blood biopsy may comprise plasma, white blood cells, red blood cells, platelets, or any combination thereof. In some cases, the health state of the one or more subjects may comprise a non-cancerous health state or cancerous health state. In some instances, the non-cancerous health state may comprise a non-cancerous disease health state or a non-diseased health state.

In some instances, receiving the fungal presence and the non-fungal microbial presence in the biological sample may comprise: (a) sequencing one or more nucleic acid molecules of the biological sample, thereby generating one or more sequencing reads; (b) aligning the one or more sequencing reads to a reference human genome library and retain one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. In some instances, aligning the one or more sequencing reads to a reference human genome library is omitted. In some cases, receiving the fungal presence and the non-fungal microbial presence in the biological sample may comprise whole genome sequencing, shotgun sequencing, target sequencing, RNA sequencing, methylation sequencing, or any combination thereof sequence of the fungal and non-fungal microbial presence nucleic acid molecules in the biological sample.

In some cases, mapping the one or more non-human sequencing reads to a fungal and non-fungal microbial reference genome library may comprise mapping to a functional genome database to generate one or more functional genomic features. In some instances, the functional genome database may comprise the Kyoto Encyclopedia of Genes and Genomes (KEGG). The one or more functional genomic features may comprise one or more metabolic features associated with one or more non-human sequencing reads. In some cases, the one or more metabolic features may comprise functional units of gene sets in metabolic pathways, functional units of gene sets that characterize phenotypic features, functional units of successive reaction steps in metabolic pathways, or any combination thereof. For example, as a result of mapping the one or more non-human sequencing reads to the KEGG database's one or more metabolic pathways, a presence and/or abundance of enzymes and/or their reaction products based on the one or more non-human sequencing reads, or any combination thereof, may be generated. In some cases, the one or more pathways may be utilized as features in addition to or in place of the one or more fungal and non-fungal microbial presence and abundance features to train a predictive model, described elsewhere herein.

In some instances, the predictive model may be configured to diagnose one or more cancers, one or more subtypes of cancer, one or more of the cancer's anatomic location, or any combination thereof. In some cases, the type of cancer may comprise bone, breast, lung, colon, brain, skin, ovary, pancreas, or any combination thereof type of cancer. In some cases, the predictive model may be configured to predict a stage of cancer, cancer prognosis, a type of cancer at a low stage (e.g., stage I or stage II cancer), a mutation status of one or more cancers, a future immunotherapy response, an optimal therapy, or any combination thereof for one or more subjects. In some instances, the predictive model may be configured to diagnose one or more stage I or stage II cancers. In some cases, the predictive model may be configured to predict one or more anatomic locations of the cancer of the subject by providing the trained predictive model an input of a non-fungal microbial presence and a fungal presence of the subject's biological sample. In some cases, the predictive model is further trained with cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof.

In some cases, the predictive model may be configured to simultaneously discriminate among one or more cancer types to diagnose a specific cancer type of the subject. In some cases, the predictive model may be configured to diagnose: adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some instances, the predictive model may be configured to diagnose one or more of the following cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some cases, removing the contaminating non-fungal microbial features and the contaminating fungal features may be completed by in silico decontamination. In some instances, removing the contaminating non-fungal microbial features and the contaminating fungal features is informed by negative experimental controls, described elsewhere herein. In some instances, removing the contaminating non-fungal microbial features and the contaminating fungal features may improve performance of the predictive model by at least 1%, at least 5%, at least 10% at least 15% or at least 20%. In some cases, the step of removing the contaminating non-fungal microbial features and the contaminated fungal features may be omitted.

In some instances, the predictive model comprises a machine learning model, regularized machine learning model, ensemble of machine learning models, or any combination thereof. In some cases, the predictive model comprises a random forest, neural network, naïve bayes, support vector machines, linear regression, k-nearest neighbors, k-means, decision tree, logistic regression, gradient boosting, or any combination thereof predictive models. In some cases, an area under a receiver operating characteristic curve of the predictive model may increase by at least 1%, at least 2%, at least 4% at least 5%, or at least 10% when the combined decontaminated fungal presence and decontaminated non-fungal presence are utilized as inputs to determine a cancer of one or more subjects.

Training a Predictive Model from a Database

Aspects of the disclosure describe a method for training a predictive model based on fungal and non-fungal microbial features to predict cancer in a subject 208, as seen in FIG. 2B. In some cases, the method may comprise: (a) receiving a fungal presence, a non-fungal microbial presence, and a health state of one or more subjects from a database 210; (b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence 212; (c) training a predictive model configured to predict cancer in a subject with the combined decontaminated fungal presence and decontaminated non-fungal microbial presence, and the corresponding health state of the one or more subjects 214. In some instances, the one or more subjects comprise non-human mammal or human subjects. In some cases, the database may comprise The Cancer Genome Atlas database (TCGA), the International Cancer Genome Consortium (ICGC) database, the Pan-Cancer Atlas of Whole Genomes (PCAWG) database, the Therapeutically Applicable Research to Generate Effective Treatments (TARGET) database, the Clinical Proteomic Tumor Analysis Consortium (CPTAC) database, the Hartwig Medical Foundation (HMF) metastasis database, the Tracking Non-Small-Cell Lung Cancer Evolution through Therapy (TRACERx) database, the 100,000 Genomes Project, or any combination thereof. In some instances, the health state of the one or more subjects comprises a non-cancerous health state or cancerous health state. In some cases, the non-cancerous health state comprises a non-cancerous disease health state or non-diseased health state.

In some cases, the non-fungal microbial presence may comprise a non-fungal microbial abundance of the biological sample from the one or more subjects. In some instances, the fungal presence may comprise a fungal abundance of the biological sample from the one or more subjects. In some instances, the fungal presence may comprise an abundance of fungal DNA, RNA, methylation, proteins, or any combination thereof. In some cases, the non-fungal microbial presence comprises an abundance of non-fungal microbial DNA, RNA, methylation, proteins, or any combination thereof.

In some cases receiving the fungal presence and the non-fungal microbial presence in the biological sample comprises: (a) sequencing one or more nucleic acid molecules of the biological sample, thereby generating one or more sequencing reads; (b) aligning the one or more sequencing reads to a reference human genome library and retain one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. In some cases, aligning the one or more sequencing reads to reference human genome library is omitted.

In some cases, the predictive model may be configured to diagnose one or more cancers, one or more subtypes of cancer, one or more of its anatomic locations, or any combination thereof. In some instances, the predictive model may be configured to predict a stage of cancer, cancer prognosis, a type of cancer at a low stage (stage I or stage II), a mutation status of one or more cancers, a future immunotherapy response, an optimal therapy, or any combination thereof for one or more subjects. In some cases, the predictive model may be configured to diagnose one or more stage I or stage II cancers. In some instances, the predictive model may be configured to simultaneously discriminate among one or more cancer types to diagnose a specific cancer type of the subject. In some cases, the type of cancer may comprise bone, breast, lung, colon, brain, skin, ovary, pancreas, or any combination thereof type of cancer. In some instances, the biological sample may comprise a tissue sample, a liquid biopsy, whole blood biopsy, or any combination thereof samples. In some cases, the liquid biopsy may comprise whole blood, red blood cells, plasma, white blood cells, saliva, urine, tears, breast milk, or any com-bination thereof. In some cases, the whole blood biopsy may comprise plasma, white blood cells, red blood cells, platelets, or any combination thereof. In some cases, mapping the one or more non-human sequencing reads to a fungal and non-fungal microbial reference genome library may comprise mapping to a functional genome database to generate one or more functional genomic features. In some instances, the functional genome database may comprise the Kyoto Encyclopedia of Genes and Genomes (KEGG). The one or more functional genomic features may comprise one or more metabolic features associated with one or more non-human sequencing reads. In some cases, the one or more metabolic features may comprise functional units of gene sets in metabolic pathways, functional units of gene sets that characterize phenotypic features, functional units of successive reaction steps in metabolic pathways, or any combination thereof. For example, as a result of mapping the one or more non-human sequencing reads to the KEGG database's one or more metabolic pathways, a presence and/or abundance of enzymes and/or their reaction products based on the one or more non-human sequencing reads, or any combination thereof, may be generated. In some cases, the one or more pathways may be utilized as features in addition to or in place of the one or more fungal and non-fungal microbial presence and abundance features to train a predictive model, described elsewhere herein.

In some cases, the predictive model comprises a machine learning model, regularized machine learning model, ensemble of machine learning models, or any combination thereof. In some instances, the predictive model comprises a random forest, neural network, naïve bayes, support vector machines, linear regression, k-nearest neighbors, k-means, decision tree, logistic regression, gradient boosting, or any combination thereof predictive model. In some instances, the predictive model is configured to predict a bodily location of a cancer of a subject by providing the trained predictive model an input of a non-fungal microbial presence and a fungal presence of the subject's biological sample. In some cases, the predictive model is further trained with cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof.

In some cases, the predictive model may be configured to diagnose adrenocortical carcinoma, bladder urothelial carcinoma, brain lower grade glioma, breast invasive carcinoma, cervical squamous cell carcinoma and endocervical adeno-carcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some cases, the predictive model may be configured to diagnose one or more of the following cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some instances, removing the contaminating non-fungal microbial features and the contaminating fungal features is completed by in silico decontamination. In some cases, removing the contaminated non-fungal microbial features and the contaminated fungal features is informed by experimental controls. In some cases, removing contaminating non-fungal microbial features and contaminating fungal features may improve performance of the predictive model by at least 1%, at least 5%, at least 10%, or at least 20%. In some cases, removing the contaminating fungal features and the contaminating non-fungal microbial features is omitted.

In some cases, receiving may comprise whole genome sequencing, shotgun sequencing, targeted sequencing, RNA sequencing, methylation sequencing, or any combination thereof sequencing of the fungal and non-fungal microbial presence nucleic acid molecules.

### Administering a Therapeutic to Treat a Cancer of a Subject

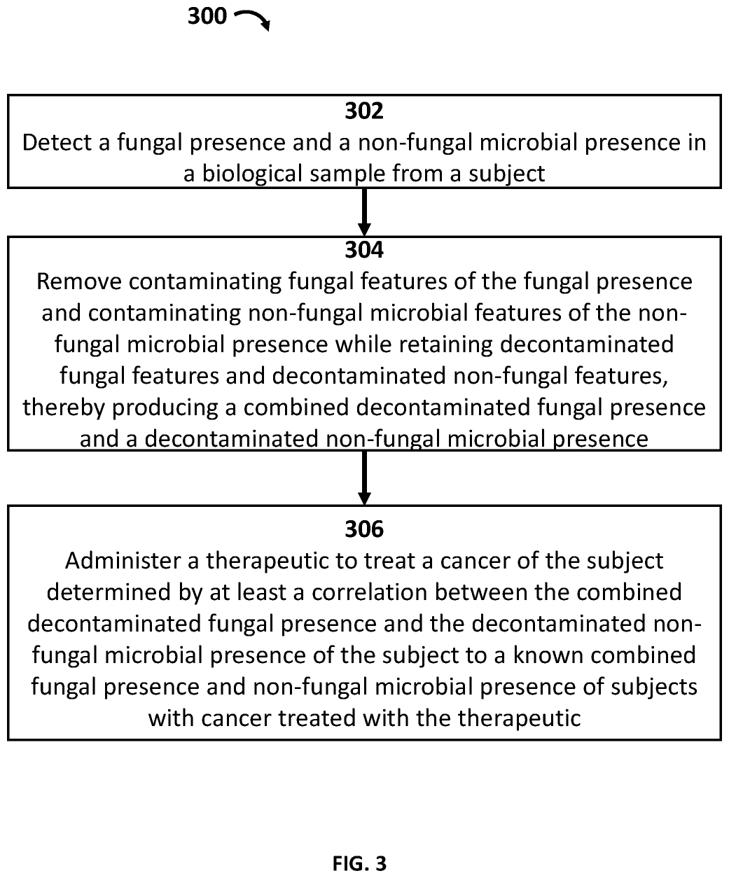

Aspects of the disclosure describe a method of treating cancer of a subject based on a combined non-fungal microbial and fungal presence of a biological sample of the subject 300, as seen in FIG. 3. In some cases, the method comprises: (a) detecting a fungal presence and a non-fungal microbial presence in a biological sample from a subject 302; (b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence 304; and (c) administering a therapeutic to treat a cancer of the subject determined by at least a correlation between the combined decontaminated fungal presence and the decontaminated non-fungal microbial presence of the subject to a known combined fungal presence and non-fungal microbial presence of subjects with cancer treated with the therapeutic 306. In some cases, the subject may comprise a non-human mammal or human subject. In some instances, the biological sample may comprise a tissue sample, a liquid biopsy, whole blood biopsy, or any combination thereof samples. In some cases, the liquid biopsy may comprise whole blood, red blood cells, plasma, white blood cells, saliva, urine, tears, breast milk, or any combination thereof. In some instances, the whole blood biopsy may comprise plasma, white blood cells, red blood cells, platelets, or any combination thereof.

In some cases, the non-fungal microbial presence may comprise a non-fungal microbial abundance of the biological sample from the one or more subjects. In some instances, the fungal presence may comprise a fungal abundance of the biological sample from the one or more subjects. In some instances, the fungal presence may comprise an abundance of fungal DNA, RNA, methylation, proteins, or any combination thereof. In some cases, the non-fungal microbial presence may comprise an abundance of non-fungal microbial DNA, RNA, methylation, proteins, or any combination thereof.

In some cases, the cancer may comprise one or more cancers, one or more subtypes of cancer, or any combination thereof. In some instances, wherein the cancer comprises a cancer at a low stage (stage I or stage II). In some instances, the cancer may comprise bone, breast, lung, colon, brain, skin, ovary, pancreas, or any combination thereof type of cancer. In some instances, the cancer may comprise adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some instances, the cancer may comprise a cancer type outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some cases, removing the contaminating non-fungal microbial features and the contaminating fungal features may be completed by in silico decontamination. In some instances, removing the contaminating non-fungal microbial features and the contaminating fungal features may be informed by experimental controls. In some instances, removing contaminating non-fungal microbial features and contaminating fungal features may improve accuracy of the predictive model by at least 1%, at least 5%, at least 10%, at least 15%, or at least 20%. In some cases, removing the contaminating non-fungal microbial features and the contaminating fungal features may be omitted.

In some instances, the correlation may be determined by a predictive model, where the predictive model may comprise a machine learning model, regularized machine learning model, ensemble of machine learning models, or any combination thereof. In some cases, the predictive model may comprise a random forest, neural network, naïve bayes, support vector machines, linear regression, k-nearest neighbors, k-means, decision tree, logistic regression, gradient boosting, or any combination thereof predictive model.

In some cases, detecting the fungal presence and the non-fungal microbial presence in the biological sample, may comprise: (a) sequencing one or more nucleic acid molecules of the biological sample, thereby generating one or more sequencing reads; (b) aligning the one or more sequencing reads to a reference human genome library and retain one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. In some cases, detecting may comprise whole genome sequencing, shotgun sequencing, targeted sequencing, RNA sequencing, methylation sequencing, or any combination thereof sequencing of the fungal and non-fungal microbial presence nucleic acid molecules.

In some cases, mapping the one or more non-human sequencing reads to a fungal and non-fungal microbial reference genome library may comprise mapping to a functional genome database to generate one or more functional genomic features. In some instances, the functional genome database may comprise the Kyoto Encyclopedia of Genes and Genomes (KEGG). The one or more functional genomic features may comprise one or more metabolic features associated with one or more non-human sequencing reads. In some cases, the one or more metabolic features may comprise functional units of gene sets in metabolic pathways, functional units of gene sets that characterize phenotypic features, functional units of successive reaction steps in metabolic pathways, or any combination thereof. For example, as a result of mapping the one or more non-human sequencing reads to the KEGG database's one or more metabolic pathways, a presence and/or abundance of enzymes and/or their reaction products based on the one or more non-human sequencing reads, or any combination thereof, may be generated. In some cases, the one or more pathways may be utilized as features in addition to or in place of the one or more fungal and non-fungal microbial presence and abundance features to train a predictive model, described elsewhere herein.

In some cases, the predictive model may be trained with one or more subject's biologic sample decontaminated fungal presence, decontaminated non-fungal microbial presence, cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof, a corresponding subject's cancer, and treatment provided to treat the subject's cancer.

In some cases, the treatment may repurpose an existing medication, which may or may not have been originally approved for targeting cancer. In some instances, the treatment comprises a small molecule, a biologic, a probiotic, a virus, a bacteriophage, immunotherapy, broad spectrum antibiotic, or any combination thereof. In some cases, the probiotic comprises an engineered bacterium strain or ensemble of engineered bacteria. In some instances, the treatment may comprise an adjuvant given in combination with a primary treatment against the cancer to improve the efficacy of the primary treatment. In some cases, the treatment may comprise adoptive cell transfer to target microbial antigens associated with the cancer or cancer microenvironment. In some instances, the treatment may comprise a cancer vaccine that exploits microbial antigens associated with the cancer or cancer microenvironment. In some instances, the treatment may comprise a monoclonal antibody against microbial antigens associated with the cancer or cancer microenvironment. In some cases, the treatment may comprise an antibody-drug conjugate designed to at least partially target microbial antigens associated with the cancer or cancer microenvironment. In some instances, the treatment may comprise a multi-valent antibody, antibody fragment, or antibody derivative thereof designed to at least partially target one or more microbial antigens associated with the cancer or cancer microenvironment. In some cases, the treatment may comprise a targeted antibiotic against a particular kind of microbe or class of functionally or biologically similar microbes. In some cases, two or more of the following treatment types are combined such that at least one type exploits the cancer microbial presence or abundance to enhance overall therapeutic efficacy: small molecules, biologics, engineered host-derived cell types, probiotics, engineered bacteria, natural-but-selective viruses, engineered viruses, and bacteriophages.

### Computer Implemented Methods for Predicting Cancer

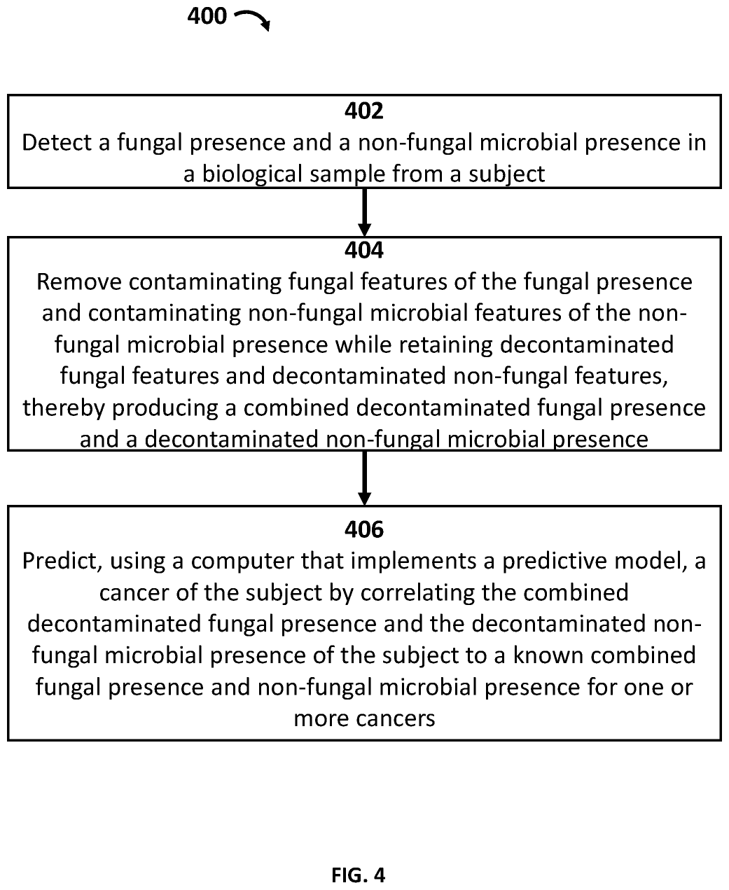

Aspects of the disclosure describe a computer-implemented method for utilizing a predictive model to predict cancer of a subject from a combined fungal and non-fungal microbial presence of a biological sample 400, as seen in FIG. 4. In some instances, the method may comprise: (a) detecting a fungal presence and a non-fungal microbial presence in a biological sample from a subject 402; (b) removing contaminating fungal features of the fungal presence and contaminating non-fungal features of the non-fungal microbial presence while retaining decontaminated fungal features and decontaminated non-fungal microbial features, thereby producing a combined decontaminated fungal presence and a decontaminated non-fungal microbial presence 404; and (c) predicting, using a computer that implements the predictive model, a cancer of the subject by correlating the combined decontaminated fungal presence and the decontaminated non-fungal microbial presence of the subject to a known combined fungal presence and non-fungal microbial presence for one or more cancers. In some instances, the subject may comprise a non-human mammal or a human subject. In some cases, the biological sample may comprise a tissue sample, a liquid biopsy, whole blood biopsy, or any combination thereof samples. In some instances, the liquid biopsy may comprise whole blood, red blood cells, plasma, white blood cells, saliva, urine, tears, breast milk, or any combination thereof. In some cases, the whole blood biopsy may comprise plasma, white blood cells, red blood cells, platelets, or any combination thereof.

In some cases, the non-fungal microbial presence may comprise bacteria, viruses, archaea, protists, or any combination thereof. In some instances, the non-fungal microbial presence may comprise a non-fungal microbial abundance of the biological sample from the subject. In some instances, the fungal presence may comprise a fungal abundance of the biological sample from the subject. In some instances, the fungal presence may comprise an abundance of fungal DNA, RNA, methylation, proteins, or any combination thereof. In some cases, the non-fungal microbial presence comprises an abundance of non-fungal microbial DNA, RNA, methylation, proteins, or any combination thereof.

In some cases, detecting the fungal presence and the non-fungal microbial presence in the biological sample may comprise: (a) sequencing one or more nucleic acid molecules of the biological sample, thereby generating one or more sequencing reads; (b) aligning the one or more sequencing reads to a reference human genome library and retaining one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. In some cases, aligning the one or more sequencing reads to the reference human genome library is omitted. In some instances, detecting may comprise whole genome sequencing, shotgun sequencing, target sequencing, RNA sequencing, methylation sequencing, or any combination thereof the one or more nucleic acid molecules of the biological sample.

In some cases, mapping the one or more non-human sequencing reads to a fungal and non-fungal microbial reference genome library may comprise mapping to a functional genome database to generate one or more functional genomic features. In some instances, the functional genome database may comprise the Kyoto Encyclopedia of Genes and Genomes (KEGG). The one or more functional genomic features may comprise one or more metabolic features associated with one or more non-human sequencing reads. In some cases, the one or more metabolic features may comprise functional units of gene sets in metabolic pathways, functional units of gene sets that characterize phenotypic features, functional units of successive reaction steps in metabolic pathways, or any combination thereof. For example, as a result of mapping the one or more non-human sequencing reads to the KEGG database's one or more metabolic pathways, a presence and/or abundance of enzymes and/or their reaction products based on the one or more non-human sequencing reads, or any combination thereof, may be generated. In some cases, the one or more pathways may be utilized as features in addition to or in place of the one or more fungal and non-fungal microbial presence and abundance features to train a predictive model, described elsewhere herein.

In some cases, removing the contaminating non-fungal microbial features and the contaminating fungal features may be completed by in silico decontamination. In some instances, removing the contaminating non-fungal microbial features and the contaminating fungal features may be informed by experimental contamination controls. In some instances, removing the contaminating non-fungal microbial features and the contaminating fungal features may improve accuracy of the predictive model by at least 1%, at least 5%, at least 10%, at least 15%, or at least 20%.

In some instances, the cancer may comprise a stage I or stage II cancer. In some cases, the cancer may comprise a bone, breast, lung, colon, brain, skin, ovary, pancreas, or any combination thereof type of cancer. In some cases, the cancer may comprise adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some instances, the cancer may comprise one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some cases, the predictive model may comprise a machine learning model, regularized machine learning model, ensemble of machine learning models, or any combination thereof. In some instances, the predictive model may comprise a random forest, neural network, naïve bayes, support vector machines, linear regression, k-nearest neighbors, k-means, decision tree, logistic regression, gradient boosting, or any combination thereof predictive model.

In some instances, predicting the cancer may further comprise predicting one or more cancers, one or more subtypes of cancer, the anatomical locations of one or more cancers, or any combination thereof. In some cases, predicting the cancer comprises predicting a stage of the cancer, cancer prognosis, a mutation status of the cancer, a future immunotherapy response of the cancer, an optimal therapy to treat the cancer, or any combination thereof for one or more subjects. In some instances, predicting the cancer may comprise predicting a cancer type among one or more cancer types. In some cases, predicting may further comprise predicting one or more anatomical locations of the cancer in the subject. In some instances, the predictive model may be further configured to receive the subject's biological sample cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof as an input to predict the cancer. In some instances, the area under a receiver operating characteristic curve of the predictive model for predicting the cancer of the subject may increase by at least 1%, at least 2%, at least 4%, at least 5%, or at least 10% when the combined decontaminated fungal presence and the decontaminated non-fungal presence is utilized during the correlation.

### Predictive Models

The methods and systems of the present disclosure may utilize or access external capabilities of artificial intelligence techniques to identify fungal and/or non-fungal microbial features to predict cancer. In some cases, the fungal and/or non-fungal microbial features may be used to train one or more predictive models, described elsewhere herein. These features may be used to accurately predict diseases or disorders (e.g., hours, days, months, or years earlier than with standard of clinical care). In some cases, the diseases or disorders may comprise cancer, as described elsewhere herein. Using such a predictive capability, health care providers (e.g., physicians) may be able to make informed, accurate risk-based decisions, thereby improving quality of care and monitoring provided to patients.

The methods and systems of the present disclosure may analyze a fungal and/or non-fungal microbial presence and/or abundance of a biological sample of a subject to determine one or more fungal features and/or non-fungal microbial features. In some cases, the methods and systems, described elsewhere herein, may train a predictive model with the one or more fungal features and/or non-fungal microbial features indicative of cancer of a subject. In some cases, the trained predictive model may then be used to generate a likelihood (e.g., a prediction) of cancer of second one or more subjects from a fungal and/or non-fungal microbial presence of the second one or more subjects' biological samples. The trained predictive model may comprise an artificial intelligence-based model, such as a machine learning based classifier, configured to process the fungal and/or non-fungal microbial presence and/or abundance data to generate the likelihood of the subject having the disease or disorder. The model may be trained using fungal and/or non-fungal microbial presence and/or abundance from one or more cohorts of patients, e.g., cancer patients receiving a treatment to train a predictive model configured to provide treatment recommendations to a patient not part of the training dataset of the predictive model. Such a predictive model may output a treatment recommendation for the patient not part of the training dataset when provided an input of the patient's fungal and/or non-fungal microbial presence and/or abundance.

The model may comprise one or more machine learning algorithms. Examples of machine learning algorithms may include a support vector machine (SVM), a naïve Bayes classification, a random forest, a neural network (such as a deep neural network (DNN), a recurrent neural network (RNN), a deep RNN, a long short-term memory (LSTM) recurrent neural network (RNN), a gated recurrent unit (GRU), a gradient boosting machine, a random forest, or other supervised learning algorithm or unsupervised machine learning, statistical, or deep learning algorithm for classification and regression. The model may likewise involve the estimation of ensemble models, comprised of multiple predictive models, and utilize techniques such as gradient boosting, for example in the construction of gradient-boosting decision trees. The model may be trained using one or more training datasets corresponding to patient data.

Training datasets may be generated from, for example, one or more cohorts of patients having common clinical disease or disorder diagnosis. Training datasets may comprise a set of fungal and/or non-fungal microbial features in the form of presence and/or abundance of the fungi and non-fungal microbes present in a biological sample of a subject. Features may comprise a corresponding cancer diagnosis of one or more subjects to aforementioned fungal and/or non-fungal microbial features. In some cases, features may comprise patient information such as patient age, patient medical history, other medical conditions, current or past medications, clinical risk scores, and time since the last observation. For example, a set of features collected from a given patient at a given time point may collectively serve as a signature, which may be indicative of a health state or status of the patient at the given time point.

Labels may comprise clinical outcomes such as, for example, a presence, absence, diagnosis, or prognosis of a disease or disorder in the subject (e.g., patient). Clinical outcomes may comprise treatment efficacy (e.g., whether a subject is a positive responder to a cancer based treatment).

Input features may be structured by aggregating the data into bins or alternatively using a one-hot encoding. Inputs may also include feature values or vectors derived from the previously mentioned inputs, such as cross-correlations.

Training records may be constructed from fungal and/or non-fungal microbial presence and/or abundance features.

The model may process the input features to generate output values comprising one or more classifications, one or more predictions, or a combination thereof. For example, such classifications or predictions may include a binary classification of a cancer or no cancer present in a subject (e.g., absence of a disease or disorder), a classification between a group of categorical labels (e.g., ‘no disease or disorder’, ‘apparent disease or disorder’, and ‘likely disease or disorder’), a likelihood (e.g., relative likelihood or probability) of developing a particular disease or disorder, a score indicative of a presence of disease or disorder, a ‘risk factor’ for the likelihood of mortality of the patient, and a confidence interval for any numeric predictions. Various machine learning techniques may be cascaded such that the output of a machine learning technique may also be used as input features to subsequent layers or subsections of the model.

In order to train the model (e.g., by determining weights and correlations of the model) to generate real-time classifications or predictions, the model can be trained using datasets. Such datasets may be sufficiently large to generate statistically significant classifications or predictions. For example, datasets may comprise: databases of data including fungal and/or non-fungal microbial presence and/or abundance of one or more subjects' biological samples.

Datasets may be split into subsets (e.g., discrete or overlapping), such as a training dataset, a development dataset, and a test dataset. For example, a dataset may be split into a training dataset comprising 80% of the dataset, a development dataset comprising 10% of the dataset, and a test dataset comprising 10% of the dataset. The training dataset may comprise about 10%, about 20%, about 30%, about 40%, about 50%, about 60%, about 70%, about 80%, or about 90% of the dataset. The development dataset may comprise about 10%, about 20%, about 30%, about 40%, about 50%, about 60%, about 70%, about 80%, or about 90% of the dataset. The test dataset may comprise about 10%, about 20%, about 30%, about 40%, about 50%, about 60%, about 70%, about 80%, or about 90% of the dataset. In some embodiments, leave one out cross validation may be employed. Training sets (e.g., training datasets) may be selected by random sampling of a set of data corresponding to one or more patient cohorts to ensure independence of sampling. Alternatively, training sets (e.g., training datasets) may be selected by proportionate sampling of a set of data corresponding to one or more patient cohorts to ensure independence of sampling.

To improve the accuracy of model predictions and reduce overfitting of the model, the datasets may be augmented to increase the number of samples within the training set. For example, data augmentation may comprise rearranging the order of observations in a training record. To accommodate datasets having missing observations, methods to impute missing data may be used, such as forward-filling, back-filling, linear interpolation, and multi-task Gaussian processes. Datasets may be filtered or batch corrected to remove or mitigate confounding factors. For example, within a database, a subset of patients may be excluded.

The model may comprise one or more neural networks, such as a neural network, a convolutional neural network (CNN), a deep neural network (DNN), a recurrent neural network (RNN), or a deep RNN. The recurrent neural network may comprise units which can be long short-term memory (LSTM) units or gated recurrent units (GRU). For example, the model may comprise an algorithm architecture comprising a neural network with a set of input features such as vital sign and other measurements, patient medical history, and/or patient demographics. Neural network techniques, such as dropout or regularization, may be used during training the model to prevent overfitting. The neural network may comprise a plurality of sub-networks, each of which is configured to generate a classification or prediction of a different type of output information (e.g., which may be combined to form an overall output of the neural network). The machine learning model may alternatively utilize statistical or related algorithms including random forest, classification and regression trees, support vector machines, discriminant analyses, regression techniques, as well as ensemble and gradient-boosted variations thereof.

When the model generates a classification or a prediction of a disease or disorder, a notification (e.g., alert or alarm) may be generated and transmitted to a health care provider, such as a physician, nurse, or other member of the patient's treating team within a hospital. Notifications may be transmitted via an automated phone call, a short message service (SMS) or multimedia message service (MMS) message, an e-mail, or an alert within a dashboard. The notification may comprise output information such as a prediction of a disease or disorder, a likelihood of the predicted disease or disorder, a time until an expected onset of the disease or disorder, a confidence interval of the likelihood or time, or a recommended course of treatment for the disease or disorder.

To validate the performance of the model, different performance metrics may be generated. For example, an area under the receiver-operating characteristic curve (AUROC) may be used to determine the diagnostic capability of the model. For example, the model may use classification thresholds which are adjustable, such that specificity and sensitivity are tunable, and the receiver-operating characteristic curve (ROC) can be used to identify the different operating points corresponding to different values of specificity and sensitivity.

In some cases, such as when datasets are not sufficiently large, cross-validation may be performed to assess the robustness of a model across different training and testing datasets.

To calculate performance metrics such as sensitivity, specificity, accuracy, positive predictive value (PPV), negative predictive value (NPV), area under the precision-recall curve (AUPR), AUROC, or similar, the following definitions may be used. A “false positive” may refer to an outcome in which a positive outcome or result has been incorrectly or prematurely generated (e.g., before the actual onset of, or without any onset of, the disease or disorder). A “true positive” may refer to an outcome in which positive outcome or result has been correctly generated, when the patient has the disease or disorder (e.g., the patient shows symptoms of the disease or disorder, or the patient's record indicates the disease or disorder). A “false negative” may refer to an outcome in which a negative outcome or result has been generated, but the patient has the disease or disorder (e.g., the patient shows symptoms of the disease or disorder, or the patient's record indicates the disease or disorder). A “true negative” may refer to an outcome in which a negative outcome or result has been generated (e.g., before the actual onset of, or without any onset of, the disease or disorder).

The model may be trained until certain pre-determined conditions for accuracy or performance are satisfied, such as having minimum desired values corresponding to diagnostic accuracy measures. For example, the diagnostic accuracy measure may correspond to prediction of a likelihood of occurrence of a disease or disorder in the subject. As another example, the diagnostic accuracy measure may correspond to prediction of a likelihood of deterioration or recurrence of a disease or disorder for which the subject has previously been treated. Examples of diagnostic accuracy measures may include sensitivity, specificity, positive predictive value (PPV), negative predictive value (NPV), accuracy, AUPR, and AUROC corresponding to the diagnostic accuracy of detecting or predicting a disease or disorder.

For example, such a pre-determined condition may be that the sensitivity of predicting the disease or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the specificity of predicting the disease or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the positive predictive value (PPV) of predicting the disease or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the negative predictive value (NPV) of predicting the disease or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the area under the curve (AUC) of a Receiver Operating Characteristic (ROC) curve (AUROC) of predicting the disease or disorder comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

As another example, such a pre-determined condition may be that the area under the precision-recall curve (AUPR) of predicting the disease or disorder comprises a value of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the disease or disorder with a sensitivity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the disease or disorder with a specificity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the disease or disorder with a positive predictive value (PPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the disease or disorder with a negative predictive value (NPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the disease or disorder with an area under the curve (AUC) of a Receiver Operating Characteristic (ROC) curve (AUROC) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the disease or disorder with an area under the precision-recall curve (AUPR) of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

The training data sets may be collected from training subjects (e.g., humans). Each training has a diagnostic status indicating that they have either been diagnosed with the biological condition, or have not been diagnosed with the biological condition.

In some embodiments, the model is a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

In some embodiments, independent component analysis (ICA) is used to de-dimensionalize the data, such as that described in Lee, T.-W. (1998): Independent component analysis: Theory and applications, Boston, Mass: Kluwer Academic Publishers, ISBN 0-7923-8261-7, and Hyvärinen, A.; Karhunen, J.; Oja, E. (2001): Independent Component Analysis, New York: Wiley, ISBN 978-0-471-40540-5, which is hereby incorporated by reference in its entirety.

In some embodiments, principal component analysis (PCA) is used to de-dimensionalize the data, such as that described in Jolliffe, I. T. (2002). Principal Component Analysis. Springer Series in Statistics. New York: Springer-Verlag. doi:10.1007/b98835. ISBN 978-0-387-95442-4, which is hereby incorporated by reference in its entirety.

SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of “kernels,” which automatically realizes a non-linear mapping to a feature space. The hyper-plane found by the SVM in feature space corresponds to a non-linear decision boundary in the input space.

Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests-Random Features,” Technical Report 567, Statistics Department, U.C. Berkeley, September 1999, which is hereby incorporated by reference in its entirety.

Clustering (e.g., unsupervised clustering model algorithms and supervised clustering model algorithms) is described on pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973. Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda 1973. Criterion functions are discussed in Section 6.8 of Duda 1973. More recently, Duda et al., Pattern Classification, 2^{nd} edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, New Jersey, each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering, where no preconceived notion of what clusters should form when the training set is clustered, are imposed.

Regression models, such as that of the multi-category logit models, are described in Agresti, An Introduction to Categorical Data Analysis, 1996, John Wiley & Sons, Inc., New York, Chapter 8, which is hereby incorporated by reference in its entirety. In some embodiments, the model makes use of a regression model disclosed in Hastie et al., 2001, The Elements of Statistical Learning, Springer-Verlag, New York, which is hereby incorporated by reference in its entirety. In some embodiments, gradient-boosting models are used toward, for example, the classification algorithms described herein; these gradient-boosting models are described in Boehmke, Bradley; Greenwell, Brandon (2019). “Gradient Boosting”. Hands-On, Machine Learning with R, Chapman & Hall. pp. 221-245 ISBN 9783-1-138-49568-5, which is hereby incorporated by reference in its entirety. In some embodiments, ensemble modeling techniques are used: these ensemble modeling techniques are described in the implementation of classification models herein, and are described in Zhou Zhihua (2012). Ensemble Methods: Foundations and Algorithms. Chapman and Hall/CRC. ISBN 978-1-439-83003-1, which is hereby incorporated by reference in its entirety.

In some embodiments, the machine learning analysis is performed by a device executing one or more programs (e.g., one or more programs stored in the Non-Persistent Memory or in Persistent Memory) including instructions to perform the data analysis. In some embodiments, the data analysis is performed by a system comprising at least one processor (e.g., a processing core) and memory (e.g., one or more programs stored in Non-Persistent Memory or in the Persistent Memory) comprising instructions to perform the data analysis.

### Computer Systems

The present disclosure provides computer systems that are programmed to implement methods of the disclosure. FIG. 12 shows a computer system 901 that is programmed or otherwise configured to predict cancer, train a predictive model, generate a recommended therapeutic, or any combination thereof methods, described elsewhere herein. The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 904 (e.g., random-access memory, read-only memory, flash memory), electronic storage unit 906 (e.g., hard disk), communication interface 908 (e.g., network adapter) for communicating with one or more other systems, and peripheral devices 907, such as cache, other memory, data storage and/or electronic display adapters. The memory 904, storage unit 906, interface 908 and peripheral devices 907 are in communication with the CPU 905 through a communication bus (solid lines), such as a motherboard. The storage unit 906 can be a data storage unit (or data repository) for storing data. The computer system 901 can be operatively coupled to a computer network (“network”) 900 with the aid of the communication interface 908. The network 900 can be the Internet, an internet and/or extranet, or an intranet and/or extranet that is in communication with the Internet. The network 900 in some cases is a telecommunication and/or data network. The network 900 can include one or more computer servers, which can enable distributed computing, such as cloud computing. The network 900, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 904. The instructions can be directed to the CPU 905, which can subsequently program or otherwise configure the CPU 905 to implement methods of the present disclosure, described elsewhere herein. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 906 can store files, such as drivers, libraries and saved programs. The storage unit 906 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 900. For instance, the computer system 901 can communicate with a remote computer system of a user. Examples of remote computer systems may include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 900.

Methods as described herein can be implemented by way of machine (e.g., computer processor) executable code stored on an electronic storage location of the computer system 901, such as, for example, on the memory 904 or electronic storage unit 906. The machine executable or machine-readable code can be provided in the form of software. During use, the code can be executed by the processor 905. In some cases, the code can be retrieved from the storage unit 906 and stored on the memory 904 for ready access by the processor 905. In some situations, the electronic storage unit 906 can be precluded, and machine-executable instructions are stored on memory 904.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 902 that comprises a user interface (UI) 903 for providing, for example, a display for visualization of prediction results or an interface for training a predictive model. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 905. The algorithm can, for example, predict cancer of a subject or subjects, determine a tailored treatment and/or therapeutic to treat a subject's or subjects' cancer, or any combination thereof.

Aspects of the disclosure describe a computer system configured to predict cancer of a subject from a combined fungal and non-fungal microbial presence of a biological sample. In some cases, the system may comprise: (a) one or more processors; and (b) a non-transient computer readable storage medium including software, where the software comprises executable instructions that, as a result of the execution, cause the one or more processors of the computer system to: (i) detect a fungal presence and a non-fungal microbial presence in a biological sample from a subject; (ii) remove contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and (iii) predict a cancer of the subject by correlating the combined decontaminated fungal presence and the decontaminated non-fungal microbial presence of the subject to a known combined fungal presence and non-fungal microbial presence for one or more cancers.

In some cases, the non-fungal microbial presence may comprise bacteria, viruses, archaea, protists, or any combination thereof. In some instances, the non-fungal microbial presence may comprise a non-fungal microbial abundance of the biological sample from the subject. In some cases, the fungal presence may comprise a fungal abundance of the biological sample from the subject. In some instances, the fungal presence may comprise an abundance of fungal DNA, RNA, methylation, proteins, or any combination thereof. In some cases, the non-fungal microbial presence may comprise an abundance of non-fungal microbial DNA, RNA, methylation, proteins, or any combination thereof.

In some cases, detecting fungal presence and the non-fungal presence in the biological sample may comprise: (a) sequencing one or more nucleic acid molecules of the biological sample, thereby generating one or more sequencing reads; (b) aligning the one or more sequencing reads to a reference human genome library and retaining one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. In some instances, aligning the one or more sequencing reads to a reference human genome library is omitted. In some cases, detecting may comprise whole genome sequencing, shotgun sequencing, targeted sequencing, RNA sequencing, methylation sequencing, or any combination thereof. In some cases, the subject may comprise a non-human mammal or a human subject. In some instances, the biological sample may comprise a tissue sample, a liquid biopsy, a whole blood biopsy, or any combination thereof samples. In some instances, the liquid biopsy may comprise whole blood, red blood cells, plasma, white blood cells, saliva, urine, tears, breast milk, or any combination thereof. In some cases, the whole blood biopsy comprises plasma, white blood cells, red blood cells, platelets, or any combination thereof.

In some cases, mapping the one or more non-human sequencing reads to a fungal and non-fungal microbial reference genome library may comprise mapping to a functional genome database to generate one or more functional genomic features. In some instances, the functional genome database may comprise the Kyoto Encyclopedia of Genes and Genomes (KEGG). The one or more functional genomic features may comprise one or more metabolic features associated with one or more non-human sequencing reads. In some cases, the one or more metabolic features may comprise functional units of gene sets in metabolic pathways, functional units of gene sets that characterize phenotypic features, functional units of successive reaction steps in metabolic pathways, or any combination thereof. For example, as a result of mapping the one or more non-human sequencing reads to the KEGG database's one or more metabolic pathways, a presence and/or abundance of enzymes and/or their reaction products based on the one or more non-human sequencing reads, or any combination thereof, may be generated. In some cases, the one or more pathways may be utilized as features in addition to or in place of the one or more fungal and non-fungal microbial presence and abundance features to train a predictive model, described elsewhere herein.

In some cases, the cancer may comprise a stage I or stage II cancer. In some instances, the cancer may comprise bone, breast, lung, colon, brain, skin, ovary, pancreas, or any combination thereof type of cancer. In some instances, the cancer may comprise: adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. In some cases, the cancer may comprise one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

In some cases, removing the contaminating non-fungal microbial features and the contaminating fungal features is completed by in silico decontamination. In some instances, removing the contaminating non-fungal microbial features and the contaminating fungal features is informed by experimental contamination controls. In some cases, removing the contaminating non-fungal microbial features and the contaminating fungal features improves accuracy of the predictive model by at least 1%, at least 5%, at least 10%, at least 15% or at least 20%. In some cases, removing the contaminating non-fungal microbial features and the contaminating fungal features is omitted.

In some instances, the predictive model may comprise a machine learning model, regularized machine learning model, ensemble of machine learning models, or any combination thereof. In some instances, the predictive model may comprise a random forest, neural network, naïve bayes, support vector machines, linear regression, k-nearest neighbors, k-means, decision tree, logistic regression, gradient boosting, or any combination thereof. In some cases, an area under a receiver operating characteristic curve of the predictive model for predicting the cancer of the subject may increase by at least 1%, at least 2%, at least 4%, at least 5%, or at least 10% when the combined decontamination fungal presence and the decontaminated non-fungal microbial presence is utilized during the correlation.

In some cases, predicting the cancer may comprise predicting one or more cancers, one or more subtypes of cancer, the anatomical location of one or more cancers, or any combination thereof in the subject. In some instances, predicting the cancer may comprise predicting a stage of the cancer, cancer prognosis, a mutation status of the cancer, a future immunotherapy response of the cancer, an optimal therapy to treat the cancer, or any combination thereof for one or more subjects. In some cases, predicting the cancer may comprise predicting a cancer type among one or more cancer types. In some instances, predicting may comprise predicting one or more anatomical locations of the cancer of the subject. In some cases, the predictive model is further configured to receive the subject's biological sample cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof as an input to predict the cancer.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### Embodiments

Numbered embodiment 1 comprises a method of predicting cancer of a subject from a combined fungal and non-fungal microbial presence of a biological sample, comprising: (a) detecting a fungal presence and a non-fungal microbial presence in a biological sample from a subject; (b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and (c) predicting a cancer of the subject by correlating the combined decontaminated fungal presence and the decontaminated non-fungal microbial presence of the subject to a known combined fungal presence and non-fungal microbial presence for one or more cancers. Numbered embodiment 2 comprises the method of embodiment 1 wherein detecting comprises whole genome sequencing, shotgun sequencing, targeted sequencing, RNA sequencing, methylation sequencing, or any combination thereof. Numbered embodiment 3 comprises the method as in embodiments 1 or 2, wherein the non-fungal microbial presence comprises bacteria, viruses, archaea, protists, or any combination thereof. Numbered embodiment 4 comprises the method as in any of embodiments 1-3, wherein the non-fungal microbial presence comprises a non-fungal microbial abundance of the biological sample from the subject. Numbered embodiment 5 comprises the method as in any of embodiments 1-4, wherein the fungal presence comprises a fungal abundance of the biological sample from the subject. Numbered embodiment 6 comprises the method as in any of embodiments 1-5, wherein predicting the cancer further comprises predicting one or more cancers, one or more subtypes of cancer, the anatomic locations of one or more cancers, or any combination thereof in the subject. Numbered embodiment 7 comprises the method as in any of embodiments 1-5, wherein predicting the cancer comprises predicting a stage of the cancer, cancer prognosis, a mutation status of the cancer, a future immunotherapy response of the cancer, an optimal therapy to treat the cancer, or any combination thereof for one or more subjects. Numbered embodiment 8 comprises the method as in any of embodiments 1-5, wherein the cancer comprises a stage I or stage II cancer. Numbered embodiment 9 comprises the method as in any of embodiments 1-5, wherein the predicting the cancer comprises simultaneously discriminating among one or more cancer types to diagnose a specific cancer type of the subject. Numbered embodiment 10 comprises the method as in any of embodiments 1-9, wherein the cancer comprises bone, breast, lung, colon, brain, skin, ovary, pancreas, or any combination thereof type of cancer. Numbered embodiment 11 comprises the method as in any of embodiments 1-9, wherein the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. Numbered embodiment 12 comprises the method as in any of embodiments 1-8, wherein cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. Numbered embodiment 13 comprises the method as in any of embodiments 1-12, wherein removing the contaminating non-fungal microbial features and the contaminating fungal features is completed by in silico decontamination. Numbered embodiment 14 comprises the method as in any of embodiments 1-12, wherein removing the contaminating non-fungal microbial features and the contaminating fungal features is informed by experimental contamination controls. Numbered embodiment 15 comprises the method as in any of embodiments 1-14, wherein predicting is conducted with a predictive model, wherein the predictive model comprises a machine learning model, regularized machine learning model, ensemble of machine learning models, or any combination thereof. Numbered embodiment 16 comprises the method as in any of embodiments 1-15, wherein the predictive model comprises a random forest, neural network, naïve bayes, support vector machines, linear regression, k-nearest neighbors, k-means, decision tree, logistic regression, gradient boosting, or any combination thereof predictive model. Numbered embodiment 17 comprises the method as in any of embodiments 1-16, wherein step (b) improves accuracy of the predictive model by at least 1%, at least 5%, at least 10%, at least 15%, or at least 20%. Numbered embodiment 18 comprises the method as in any of embodiments 1-16, wherein step (b) is omitted. Numbered embodiment 19 comprises the method as in any of embodiments 1-18, wherein the subject comprises a non-human mammal or a human subject. Numbered embodiment 20 comprises the method as in any of embodiments 1-19, wherein the biological sample comprises a tissue sample, a liquid biopsy, whole blood biopsy, or any combination thereof samples. Numbered embodiment 21 comprises the method as in any of embodiments 1-20, wherein the liquid biopsy comprises whole blood, red blood cells, plasma, white blood cells, saliva, urine, tears, breast milk, or any combination thereof. Numbered embodiment 22 comprises the method of embodiment 20, wherein the whole blood biopsy comprises plasma, white blood cells, red blood cells, platelets, or any combination thereof. Numbered embodiment 23 comprises the method as in any of embodiments 1-22, wherein the fungal presence comprises an abundance of fungal DNA, RNA, methylation, proteins, or any combination thereof. Numbered embodiment 24 comprises the method as in any of embodiments 1-23, wherein the non-fungal microbial presence comprises an abundance of non-fungal microbial DNA, RNA, methylation, proteins, or any combination thereof. Numbered embodiment 25 comprises the method as in any of embodiments 1-24, wherein detecting the fungal presence and the non-fungal microbial presence in the biological sample comprises: (a) sequencing one or more nucleic acid molecules of the biological sample, thereby generating one or more sequencing reads; (b) aligning the one or more sequencing reads to a reference human genome library and retaining one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. Numbered embodiment 26 comprises the method as in any of embodiments 1-25, wherein aligning the one or more sequencing reads to a reference human genome library is omitted. Numbered embodiment 27 comprises the method as in any of embodiments 1-26, wherein predicting further comprises predicting one or more anatomic locations of the cancer of the subject. Numbered embodiment 28 comprises the method as in any of embodiments 1-27, wherein the predictive model is further configured to receive the subject's biological sample cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof as an input to predict the cancer. Numbered embodiment 29 comprises the method as in any of embodiments 1-28, wherein an area under a receiver operating curve of the predictive model is increased by at least 1%, at least 2%, at least 4%, at least 5%, or at least 10% when the combined decontaminated fungal presence and the decontaminated non-fungal presence is utilized during the correlation.

Numbered embodiment 30 comprises a method for training a predictive model based on fungal and non-fungal microbial features to diagnose cancer in a subject, comprising: (a) receiving, from a biological sample of one or more subjects, a fungal presence, a non-fungal microbial presence, and a corresponding health state of the one or more subjects; (b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and (c) training a predictive model with the combined decontaminated fungal presence and the decontaminated non-fungal microbial presence, and the corresponding health state of the one or more subjects. Numbered embodiment 31 comprises the method of embodiment 30, wherein the non-fungal microbial presence comprises a non-fungal microbial abundance of the biological sample from the one or more subjects. Numbered embodiment 32 comprises the method as in embodiments 30 or 31, wherein the fungal presence comprises a fungal abundance of the biological sample from the one or more subjects. Numbered embodiment 33 comprises the method as in any of embodiments 30-32, wherein the predictive model is configured to diagnose one or more cancers, one or more subtypes of cancer, one or more of the cancer's anatomic locations, or any combination thereof. Numbered embodiment 34 comprises the method as in any of embodiments 30-32, wherein the predictive model is configured to predict a stage of cancer, cancer prognosis, a type of cancer at a low stage (stage I or stage II), a mutation status of one or more cancers, a future immunotherapy response, an optimal therapy, or any combination thereof for one or more subjects. Numbered embodiment 35 comprises the method as in any of embodiments 30-32, wherein the predictive model is configured to diagnose one or more stage I or stage II cancers of one or more subjects. Numbered embodiment 36 comprises the method as in any of embodiments 30-32, wherein the predictive model is configured to simultaneously discriminate among one or more cancer types to diagnose a specific cancer type of the subject. Numbered embodiment 37 comprises the method as in any of embodiments 30-36, wherein the associated type of cancer comprises bone, breast, lung, colon, brain, skin, ovary, pancreas, or any combination thereof type of cancer. Numbered embodiment 38 comprises the method as in any of embodiments 30-37, wherein the predictive model is configured to diagnose adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. Numbered embodiment 39 comprises the method as in any of embodiments 30-37, wherein the predictive model is configured to diagnose one or more of the following cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. Numbered embodiment 40 comprises the method as in any of embodiments 30-39, wherein removing the contaminating non-fungal microbial features and the contaminating fungal features is completed by in silico decontamination. Numbered embodiment 41 comprises the method as in any of embodiments 30-39, wherein removing the contaminating non-fungal microbial features and the contaminating fungal features is informed by negative experimental controls. Numbered embodiment 42 comprises the method as in any of embodiments 30-41, wherein the predictive model comprises a machine learning model, regularized machine learning model, ensemble of machine learning models, or any combination thereof. Numbered embodiment 43 comprises the method as in any of embodiments 30-42, wherein the predictive model comprises a random forest, neural network, naïve bayes, support vector machines, linear regression, k-nearest neighbors, k-means, decision tree, logistic regression, gradient boosting, or any combination thereof predictive model. Numbered embodiment 44 comprises the method as in any of embodiments 30-43, wherein step (b) improves accuracy of the predictive model by at least 1%, at least 5%, at least 10%, at least 15%, or at least 20%. Numbered embodiment 45 comprises the method as in any of embodiments 30-43, wherein step (b) is omitted. Numbered embodiment 46 comprises the method as in any of embodiments 30-45, wherein the one or more subjects comprise non-human mammal or human subjects. Numbered embodiment 47 comprises the method as in any of embodiments 30-46, wherein the biological sample comprises a tissue sample, a liquid biopsy, whole blood biopsy, or any combination thereof samples. Numbered embodiment 48 comprises the method as in any of embodiments 30-47, wherein the liquid biopsy comprises whole blood, red blood cells, plasma, white blood cells, saliva, urine, tears, breast milk, or any combination thereof. Numbered embodiment 49 comprises the method of embodiment 47, wherein the whole blood biopsy comprises plasma, white blood cells, red blood cells, platelets, or any combination thereof. Numbered embodiment 50 comprises the method as in any of embodiments 30-49, wherein the fungal presence comprises an abundance of fungal DNA, RNA, methylation, proteins, or any combination thereof. Numbered embodiment 51 comprises the method as in any of embodiments 30-50, wherein the non-fungal microbial presence comprises an abundance of non-fungal microbial DNA, RNA, methylation, proteins, or any combination thereof. Numbered embodiment 52 comprises the method as in any of embodiments 30-51, wherein detecting the fungal presence and the non-fungal microbial presence in the biological sample comprises: (a) sequencing one or more nucleic acid molecules of the biological sample, thereby generating one or more sequencing reads; (b) aligning the one or more sequencing reads to a reference human genome library and retain one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. Numbered embodiment 53 comprises the method as in any of embodiments 30-52, wherein aligning the one or more sequencing reads to a reference human genome library is omitted. Numbered embodiment 54 comprises the method as in any of embodiments 30-52, wherein predictive model is configured to predict one or more anatomic locations of a cancer of a subject by providing the trained predictive model an input of a non-fungal microbial presence and a fungal presence of the subject's biological sample. Numbered embodiment 55 comprises the method as in any of embodiments 30-54, wherein the predictive model is further trained with cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof. Numbered embodiment 56 comprises the method as in any of embodiments 30-55, wherein receiving comprises whole genome sequencing, shotgun sequencing, targeted sequencing, RNA sequencing, methylation sequencing, or any combination thereof sequencing of the fungal and non-fungal microbial presence nucleic acid molecules in the biological sample. Numbered embodiment 57 comprises the method as in any of embodiments 30-56, wherein the health state of the one or more subjects comprises a non-cancerous health state or cancerous health state. Numbered embodiment 58 comprises the method as in any of embodiments 30-57, wherein the non-cancerous health state comprises a non-cancerous disease health state or a non-diseased health state

Numbered embodiment 59 comprises a method for training a predictive model based on fungal and non-fungal microbial features to predict cancer in a subject, comprising: (a) receiving a fungal presence, a non-fungal microbial presence, and a health state of one or more subjects from a database; (b) removing contaminating fungal features of the fungal presence and contaminating non-fungal microbial features the non-fungal microbial presence while retaining decontaminated fungal features and decontaminated non-fungal microbial features, thereby producing a combined decontaminated fungal presence and a decontaminated non-fungal microbial presence; and (c) training a predictive model configured to predict cancer in a subject with the combined decontaminated fungal presence and decontaminated non-fungal microbial presence, and the corresponding health state of the one or more subjects. Numbered embodiment 60 comprises the method of embodiment 59, wherein the non-fungal microbial presence comprises a non-fungal microbial abundance of the biological sample from the one or more subjects. Numbered embodiment 61 comprises the method as in embodiments 59 or 60, wherein the fungal presence comprises a fungal abundance of the biological sample from the one or more subjects. Numbered embodiment 62 comprises the method as in any of embodiments 59-61, wherein the predictive model is configured to diagnose one or more cancers, one or more subtypes of cancer, one or more of its anatomic locations, or any combination thereof. Numbered embodiment 63 comprises the method as in any of embodiments 59-61, wherein the predictive model is configured to predict a stage of cancer, cancer prognosis, a type of cancer stage I or stage II, a mutation status of one or more cancers, a future immunotherapy response, an optimal therapy, or any combination thereof for one or more subjects. Numbered embodiment 64 comprises the method as in any of embodiments 59-61, wherein the predictive model is configured to diagnose one or more stage I or stage II cancers of one or more subjects. Numbered embodiment 65 comprises the method as in any of embodiments 59-61, wherein the predictive model is configured to simultaneously discriminate among one or more cancer types to diagnose a specific cancer type of the subject. Numbered embodiment 66 comprises the method as in any of embodiments 59-65, wherein the associated type of cancer comprises bone, breast, lung, colon, brain, skin, ovary, pancreas, or any combination thereof type of cancer. Numbered embodiment 67 comprises the method as in any of embodiments 59-66, wherein the predictive model is configured to diagnose adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. Numbered embodiment 68 comprises the method as in any of embodiments 59-66, wherein the predictive model is configured to diagnose one or more of the following cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. Numbered embodiment 69 comprises the method as in any of embodiments 59-68, wherein removing the contaminating non-fungal microbial features and the contaminating fungal features is completed by in silico decontamination. Numbered embodiment 70 comprises the method as in any of embodiments 59-68, wherein removing the contaminated non-fungal microbial features and the contaminated fungal features is informed by experimental controls. Numbered embodiment 71 comprises the method as in any of embodiments 59-70, wherein the predictive model comprises a machine learning model, regularized machine learning model, ensemble of machine learning models, or any combination thereof. Numbered embodiment 72 comprises the method as in any of embodiments 59-71, wherein the predictive model comprises a random forest, neural network, naïve bayes, support vector machines, linear regression, k-nearest neighbors, k-means, decision tree, logistic regression, gradient boosting, or any combination thereof predictive model. Numbered embodiment 73 comprises the method as in any of embodiments 59-72, wherein step (b) improves accuracy of the predictive model by at least 1%, at least 5%, at least 10%, at least 15%, or at least 20%. Numbered embodiment 74 comprises the method as in any of embodiments 59-72, wherein step (b) is omitted. Numbered embodiment 75 comprises the method as in any of embodiments 59-74, wherein the one or more subjects comprise non-human mammal or human subjects. Numbered embodiment 76 comprises the method as in any of embodiments 59-75, wherein the biological sample comprises a tissue sample, a liquid biopsy, whole blood biopsy, or any combination thereof samples. Numbered embodiment 77 comprises the method as in any of embodiments 59-76, wherein the liquid biopsy comprises whole blood, red blood cells, plasma, white blood cells, saliva, urine, tears, breast milk, or any combination thereof. Numbered embodiment 78 comprises the method of embodiment 76, wherein the whole blood biopsy comprises plasma, white blood cells, red blood cells, platelets, or any combination thereof. Numbered embodiment 79 comprises the method as in any of embodiments 59-78, wherein the fungal presence comprises an abundance of fungal DNA, RNA, methylation, proteins, or any combination thereof. Numbered embodiment 80 comprises the method as in any of embodiments 59-79, wherein the non-fungal microbial presence comprises an abundance of non-fungal microbial DNA, RNA, methylation, proteins, or any combination thereof. Numbered embodiment 81 comprises the method as in any of embodiments 59-80, wherein detecting the fungal presence and the non-fungal microbial presence in the biological sample comprises: (a) sequencing one or more nucleic acid molecules of the biological sample, thereby generating one or more sequencing reads; (b) aligning the one or more sequencing reads to a reference human genome library and retain one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. Numbered embodiment 82 comprises the method as in any of embodiments 59-81, wherein aligning the one or more sequencing reads to reference human genome library is omitted. Numbered embodiment 83 comprises the method as in any of embodiments 59-81, wherein predictive model is configured to predict an anatomic location of a cancer of a subject by providing the trained predictive model an input of a non-fungal microbial presence and a fungal presence of the subject's biological sample. Numbered embodiment 84 comprises the method as in any of embodiments 59-83, wherein the predictive model is further trained with cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof. Numbered embodiment 85 comprises the method as in any of embodiments 59-84, wherein detecting comprises whole genome sequencing, shotgun sequencing, targeted sequencing, RNA sequencing, methylation sequencing, or any combination thereof sequencing of the fungal and non-fungal microbial presence nucleic acid molecules. Numbered embodiment 86 comprises the method as in any of embodiments 59-85, wherein the database comprises The Cancer Genome Atlas database (TCGA), the International Cancer Genome Consortium (ICGC) database, the Pan-Cancer Atlas of Whole Genomes (PCAWG) database, the Therapeutically Applicable Research to Generate Effective Treatments (TARGET) database, the Clinical Proteomic Tumor Analysis Consortium (CPTAC) database, the Hartwig Medical Foundation (HMF) metastasis database, the Tracking Non-Small-Cell Lung Cancer Evolution through Therapy (TRACERx) database, the 100,000 Genomes Project, or any combination thereof. Numbered embodiment 87 comprises the method as in any of embodiments 59-86, wherein the health state of the one or more subjects comprises a non-cancerous health state or cancerous health state. Numbered embodiment 88 comprises the method as in any of embodiments 59-87, wherein the non-cancerous health state comprises a non-cancerous disease health state or a non-diseased health state.

Numbered embodiment 89 comprises a method of treating cancer of a subject based on a combined non-fungal microbial and fungal presence of a biological sample of the subject, comprising: (a) detecting a fungal presence and a non-fungal microbial presence in a biological sample from a subject; (b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and (c) administering a therapeutic to treat a cancer of the subject determined by at least a correlation between the combined decontaminated fungal presence and the decontaminated non-fungal microbial presence of the subject to a known combined fungal presence and non-fungal microbial presence of subjects with cancer treated with the therapeutic. Numbered embodiment 90 comprises the method of embodiment 89, wherein the non-fungal microbial presence comprises a non-fungal microbial abundance of the biological sample from the one or more subjects. Numbered embodiment 91 comprises the method as in embodiments 89 or 90, wherein the fungal presence comprises a fungal abundance of the biological sample from the one or more subjects. Numbered embodiment 92 comprises the method as in any of embodiments 89-91, wherein the cancer of the comprises one or more cancers, one or more subtypes of cancer, or any combination thereof. Numbered embodiment 93 comprises the method as in any of embodiments 89-91, wherein the cancer comprises a cancer at a low stage (stage I or stage II). Numbered embodiment 94 comprises the method as in any of embodiments 89-93, wherein the cancer comprises bone, breast, lung, colon, brain, skin, ovary, pancreas, or any combination thereof type of cancer. Numbered embodiment 95 comprises the method as in any of embodiments 89-94, wherein the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. Numbered embodiment 96 comprises the method as in any of embodiments 89-94, wherein the cancer comprises a cancer type outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. Numbered embodiment 97 comprises the method as in any of embodiments 89-96, wherein removing the contaminating non-fungal microbial features and the contaminating fungal features is completed by in silico decontamination. Numbered embodiment 98 comprises the method as in any of embodiments 89-96, wherein removing the contaminated non-fungal microbial features and the contaminated fungal features is informed by experimental controls. Numbered embodiment 99 comprises the method as in any of embodiments 89-98, wherein the correlation is determined by a predictive model, wherein the predictive model comprises a machine learning model, regularized machine learning model, ensemble of machine learning models, or any combination thereof. Numbered embodiment 100 comprises the method as in any of embodiments 89-99, wherein the predictive model comprises a random forest, neural network, naïve bayes, support vector machines, linear regression, k-nearest neighbors, k-means, decision tree, logistic regression, gradient boosting, or any combination thereof predictive model. Numbered embodiment 101 comprises the method as in any of embodiments 89-100, wherein step (b) improves accuracy of the predictive model by at least 1%, at least 5%, at least 10%, at least 15%, or at least 20%. Numbered embodiment 102 comprises the method as in any of embodiments 89-100, wherein step (b) is omitted. Numbered embodiment 103 comprises the method as in any of embodiments 89-102, wherein the subject comprises a non-human mammal or human subject. Numbered embodiment 104 comprises the method as in any of embodiments 89-103, wherein the biological sample comprises a tissue sample, a liquid biopsy, whole blood biopsy, or any combination thereof samples. Numbered embodiment 105 comprises the method as in any of embodiments 89-104, wherein the liquid biopsy comprises whole blood, red blood cells, plasma, white blood cells, saliva, urine, tears, breast milk, or any combination thereof. Numbered embodiment 106 comprises the method of embodiment 104, wherein the whole blood biopsy comprises plasma, white blood cells, red blood cells, platelets, or any combination thereof. Numbered embodiment 107 comprises the method as in any of embodiments 89-106, wherein the fungal presence comprises an abundance of fungal DNA, RNA, methylation, proteins, or any combination thereof. Numbered embodiment 108 comprises the method as in any of embodiments 89-107, wherein the non-fungal microbial presence comprises an abundance of non-fungal microbial DNA, RNA, methylation, proteins, or any combination thereof. Numbered embodiment 109 comprises the method as in any of embodiments 89-108, wherein detecting the fungal presence and the non-fungal microbial presence in the biological sample comprises: (a) sequencing one or more nucleic acid molecules of the biological sample, thereby generating one or more sequencing reads; (b) aligning the one or more sequencing reads to a reference human genome library and retain one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. Numbered embodiment 110 comprises the method as in any of embodiments 89-109, wherein the predictive model is trained with one or more subject's biologic sample decontaminated fungal presence, decontaminated non-fungal microbial presence, cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof, a corresponding subject's cancer, and treatment provided to treat the subject's cancer. Numbered embodiment 111 comprises the method as in any of embodiments 89-110, wherein detecting comprises whole genome sequencing, shotgun sequencing, targeted sequencing, RNA sequencing, methylation sequencing, or any combination thereof sequencing of the fungal and non-fungal microbial presence nucleic acid molecules. Numbered embodiment 112 comprises the method as in any of embodiments 89-111, wherein the treatment repurposes an existing medication, which may or may not have been originally approved for targeting cancer. Numbered embodiment 113 comprises the method as in any of embodiments 89-112, wherein the treatment comprises a small molecule, a biologic, a probiotic, a virus, a bacteriophage, immunotherapy, broad spectrum antibiotic, or any combination thereof. Numbered embodiment 114 comprises the method as in any of embodiments 89-113, wherein the probiotic comprises an engineered bacterium strain or ensemble of engineered bacteria. Numbered embodiment 115 comprises the method as in any of embodiments 89-112, wherein the treatment comprises an adjuvant given in combination with a primary treatment against the cancer to improve the efficacy of the primary treatment. Numbered embodiment 116 comprises the method as in any of embodiments 89-112, wherein the treatment comprises adoptive cell transfer to target microbial antigens associated with the cancer or cancer microenvironment. Numbered embodiment 117 comprises the method as in any of embodiments 89-112, wherein the treatment comprises a cancer vaccine that exploits microbial antigens associated with the cancer or cancer microenvironment. Numbered embodiment 118 comprises the method as in any of embodiments 89-112, wherein the treatment comprises a monoclonal antibody against microbial antigens associated with the cancer or cancer microenvironment. Numbered embodiment 119 comprises the method as in any of embodiments 89-112, wherein the treatment comprises an antibody-drug conjugate designed to at least partially target microbial antigens associated with the cancer or cancer microenvironment. Numbered embodiment 120 comprises the method as in any of embodiments 89-112, wherein the treatment comprises a multi-valent antibody, antibody fragment, or antibody derivative thereof designed to at least partially target one or more microbial antigens associated with the cancer or cancer microenvironment. Numbered embodiment 121 comprises the method as in any of embodiments 89-112, wherein the treatment comprises a targeted antibiotic against a particular kind of microbe or class of functionally or biologically similar microbes. Numbered embodiment 122 comprises the method as in any of embodiments 89-112, wherein two or more of the following treatment types are combined such that at least one type exploits the cancer microbial presence or abundance to enhance overall therapeutic efficacy: small molecules, biologics, engineered host-derived cell types, probiotics, engineered bacteria, natural-but-selective viruses, engineered viruses, and bacteriophages.

Numbered embodiment 123 comprises a computer-implemented method for utilizing a predictive model to predict cancer of a subject from a combined fungal and non-fungal microbial presence of a biological sample, comprising: (a) detecting a fungal presence and a non-fungal microbial presence in a biological sample from a subject; (b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and (c) predicting, using a computer that implements the predictive model, a cancer of the subject by correlating the combined decontaminated fungal presence and the decontaminated non-fungal microbial presence of the subject to a known combined fungal presence and non-fungal microbial presence for one or more cancers. Numbered embodiment 124 comprises the computer-implemented method of embodiment 123, wherein detecting comprises whole genome sequencing, shotgun sequencing, targeted sequencing, RNA sequencing, methylation sequencing, or any combination thereof. Numbered embodiment 125 comprises the computer-implemented method as in embodiments 123 or 124, wherein the non-fungal microbial presence comprises bacteria, viruses, archaea, protists, or any combination thereof. Numbered embodiment 126 comprises the computer-implemented method as in any of embodiments 123-125, wherein the non-fungal microbial presence comprises a non-fungal microbial abundance of the biological sample from the subject. Numbered embodiment 127 comprises the computer-implemented method as in any of embodiments 123-126, wherein the fungal presence comprises a fungal abundance of the biological sample from the subject. Numbered embodiment 128 comprises the computer-implemented method as in any of embodiments 123-127, wherein predicting the cancer further comprises predicting one or more cancers, one or more subtypes of cancer, the anatomic locations of one or more cancers, or any combination thereof in the subject. Numbered embodiment 129 comprises the computer-implemented method as in any of embodiments 123-127, wherein predicting the cancer comprises predicting a stage of the cancer, cancer prognosis, a mutation status of the cancer, a future immunotherapy response of the cancer, an optimal therapy to treat the cancer, or any combination thereof for one or more subjects. Numbered embodiment 130 comprises the computer-implemented method as in any of embodiments 123-127, wherein the cancer comprises a stage I or stage II cancer. Numbered embodiment 131 comprises the computer-implemented method as in any of embodiments 123-127, wherein the predicting the cancer comprises simultaneously discriminating among one or more cancer types to diagnose a specific cancer type of the subject. Numbered embodiment 132 comprises the computer-implemented method as in any of embodiments 123-131, wherein the cancer comprises bone, breast, lung, colon, brain, skin, ovary, pancreas, or any combination thereof type of cancer. Numbered embodiment 133 comprises the computer-implemented method as in any of embodiments 123-132, wherein the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. Numbered embodiment 134 comprises the computer-implemented method as in any of embodiments 123-132, wherein cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. Numbered embodiment 135 comprises the computer-implemented method as in any of embodiments 123-134, wherein removing the contaminating non-fungal microbial features and the contaminating fungal features is completed by in silico decontamination. Numbered embodiment 136 comprises the computer-implemented method as in any of embodiments 123-134, wherein removing the contaminating non-fungal microbial features and the contaminating fungal features is informed by experimental contamination controls. Numbered embodiment 137 comprises the computer-implemented method as in any of embodiments 123-136, wherein the predictive model comprises a machine learning model, regularized machine learning model, ensemble of machine learning models, or any combination thereof. Numbered embodiment 138 comprises the computer-implemented method as in any of embodiments 123-137, wherein the predictive model comprises a random forest, neural network, naïve bayes, support vector machines, linear regression, k-nearest neighbors, k-means, decision tree, logistic regression, gradient boosting, or any combination thereof predictive model. Numbered embodiment 139 comprises the computer-implemented method as in any of embodiments 123-138, wherein step (b) improves accuracy of the predictive model by at least 1%, at least 5%, at least 10%, at least 15%, or at least 20%. Numbered embodiment 140 comprises the computer-implemented method as in any of embodiments 123-139, wherein step (b) is omitted. Numbered embodiment 141 comprises the computer-implemented method as in any of embodiments 123-140, wherein the subject comprises a non-human mammal or a human subject. Numbered embodiment 142 comprises the computer-implemented method as in any of embodiments 123-141, wherein the biological sample comprises a tissue sample, a liquid biopsy, whole blood biopsy, or any combination thereof samples. Numbered embodiment 143 comprises the computer-implemented method as in any of embodiments 123-142, wherein the liquid biopsy comprises whole blood, red blood cells, plasma, white blood cells, saliva, urine, tears, breast milk, or any combination thereof. Numbered embodiment 144 comprises the computer-implemented method as in any of embodiments 123-143, wherein the whole blood biopsy comprises plasma, white blood cells, red blood cells, platelets, or any combination thereof. Numbered embodiment 145 comprises the computer-implemented method as in any of embodiments 123-144, wherein the fungal presence comprises an abundance of fungal DNA, RNA, methylation, proteins, or any combination thereof. Numbered embodiment 146 comprises the computer-implemented method as in any of embodiments 123-145, wherein the non-fungal microbial presence comprises an abundance of non-fungal microbial DNA, RNA, methylation, proteins, or any combination thereof. Numbered embodiment 147 comprises the computer-implemented method as in any of embodiments 123-146, wherein detecting the fungal presence and the non-fungal microbial presence in the biological sample comprises: (a) sequencing one or more nucleic acid molecules of the biological sample, thereby generating one or more sequencing reads; (b) aligning the one or more sequencing reads to a reference human genome library and retaining one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. Numbered embodiment 148 comprises the computer-implemented method as in any of embodiments 123-147, wherein aligning the one or more sequencing reads to a reference human genome library is omitted. Numbered embodiment 149 comprises the computer-implemented method as in any of embodiments 123-148, wherein predicting further comprises predicting one or more anatomic locations of the cancer of the subject. Numbered embodiment 150 comprises the computer-implemented method as in any of embodiments 123-149, wherein the predictive model is further configured to receive the subject's biological sample cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof as an input to predict the cancer. Numbered embodiment 151 comprises the computer-implemented method as in any of embodiments 123-150, wherein detecting comprises whole genome sequencing, shotgun sequencing, targeted sequencing, RNA sequencing, methylation sequencing, or any combination thereof the one or more nucleic acid molecules of the biological sample. Numbered embodiment 152 comprises the computer-implemented method as in any of embodiments 123-151, wherein an area under a receiver operating curve of the predictive model for predicting the cancer of the subject is increased by at least 1%, at least 2%, at least 4%, at least 5%, or at least 10% when the combined decontaminated fungal presence and the decontaminated non-fungal presence is utilized during the correlation.

Numbered embodiment 153 comprises a computer system configured to predict cancer of a subject from a combined fungal and non-fungal microbial presence of a biological sample, comprising: (a) one or more processors; and (b) a non-transient computer readable storage medium including software, wherein the software comprises executable instructions that, as a result of the execution, cause the one or more processors of the computer system to: (i) detect a fungal presence and a non-fungal microbial presence in a biological sample from a subject; (ii) remove contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and (iii) predict a cancer of the subject by correlating the combined decontaminated fungal presence and the decontaminated non-fungal microbial presence of the subject to a known combined fungal presence and non-fungal microbial presence for one or more cancers. Numbered embodiment 154 comprises the computer system of embodiment 153, wherein detecting comprises whole genome sequencing, shotgun sequencing, targeted sequencing, RNA sequencing, methylation sequencing, or any combination thereof. Numbered embodiment 155 comprises the computer system as in embodiments 153 or 154, wherein the non-fungal microbial presence comprises bacteria, viruses, archaea, protists, or any combination thereof. Numbered embodiment 156 comprises the computer system as in any of embodiments 153-155, wherein the non-fungal microbial presence comprises a non-fungal microbial abundance of the biological sample from the subject. Numbered embodiment 157 comprises the computer system as in any of embodiments 153-156, wherein the fungal presence comprises a fungal abundance of the biological sample from the subject. Numbered embodiment 158 comprises the computer system as in any of embodiments 153-157, wherein predicting the cancer further comprises predicting one or more cancers, one or more subtypes of cancer, the anatomic locations of one or more cancers, or any combination thereof in the subject. Numbered embodiment 159 comprises the computer system as in any of embodiments 153-157, wherein predicting the cancer comprises predicting a stage of the cancer, cancer prognosis, a mutation status of the cancer, a future immunotherapy response of the cancer, an optimal therapy to treat the cancer, or any combination thereof for one or more subjects. Numbered embodiment 160 comprises the computer system as in any of embodiments 153-157, wherein the cancer comprises a stage I or stage II cancer. Numbered embodiment 161 comprises the computer system as in any of embodiments 153-157, wherein the predicting the cancer comprises predicting a cancer type among one or more cancer types. Numbered embodiment 162 comprises the computer system as in any of embodiments 153-161, wherein the cancer comprises bone, breast, lung, colon, brain, skin, ovary, pancreas, or any combination thereof type of cancer. Numbered embodiment 163 comprises the computer system as in any of embodiments 153-161, wherein the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers. Numbered embodiment 164 comprises the computer system as in any of embodiments 153-161, wherein cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers. Numbered embodiment 165 comprises the computer system as in any of embodiments 153-164, wherein removing the contaminating non-fungal microbial features and the contaminating fungal features is completed by in silico decontamination. Numbered embodiment 166 comprises the computer system as in any of embodiments 153-164, wherein removing the contaminating non-fungal microbial features and the contaminating fungal features is informed by experimental contamination controls. Numbered embodiment 167 comprises the computer system as in any of embodiments 153-166, wherein the predictive model comprises a machine learning model, regularized machine learning model, ensemble of machine learning models, or any combination thereof. Numbered embodiment 168 comprises the computer system as in any of embodiments 153-167, wherein the predictive model comprises a random forest, neural network, naïve bayes, support vector machines, linear regression, k-nearest neighbors, k-means, decision tree, logistic regression, gradient boosting, or any combination thereof predictive model. Numbered embodiment 169 comprises the computer system as in any of embodiments 153-168, wherein step (b) improves accuracy of the predictive model by at least 1%, at least 5%, at least 10%, at least 15%, or at least 20%. Numbered embodiment 170 comprises the computer system as in any of embodiments 153-168, wherein step (b) is omitted. Numbered embodiment 171 comprises the computer system as in any of embodiments 153-170, wherein the subject comprises a non-human mammal or a human subject. Numbered embodiment 172 comprises the computer system as in any of embodiments 153-171, wherein the biological sample comprises a tissue sample, a liquid biopsy, whole blood biopsy, or any combination thereof samples. Numbered embodiment 173 comprises the computer system as in any of embodiments 153-172, wherein the liquid biopsy comprises whole blood, red blood cells, plasma, white blood cells, saliva, urine, tears, breast milk, or any combination thereof. Numbered embodiment 174 comprises the computer system as in any of embodiments 153-173, wherein the whole blood biopsy comprises plasma, white blood cells, red blood cells, platelets, or any combination thereof. Numbered embodiment 175 comprises the computer system as in any of embodiments 153-174, wherein the fungal presence comprises an abundance of fungal DNA, RNA, methylation, proteins, or any combination thereof. Numbered embodiment 176 comprises the computer system as in any of embodiments 153-175, wherein the non-fungal microbial presence comprises an abundance of non-fungal microbial DNA, RNA, methylation, proteins, or any combination thereof. Numbered embodiment 177 comprises the computer system as in any of embodiments 153-176, wherein detecting the fungal presence and the non-fungal microbial presence in the biological sample comprises: (a) sequencing one or more nucleic acid molecules of the biological sample, thereby generating one or more sequencing reads; (b) aligning the one or more sequencing reads to a reference human genome library and retaining one or more non-human sequencing reads that do not align to the reference human genome library; and (c) mapping the one or more non-human sequencing reads to a fungal and non-fungal microbial reference genome library thereby generating a fungal presence and a non-fungal microbial presence of the biological sample. Numbered embodiment 178 comprises the computer system as in any of embodiments 153-177, wherein aligning the one or more sequencing reads to a reference human genome library is omitted. Numbered embodiment 179 comprises the computer system as in any of embodiments 153-178, wherein predicting further comprises predicting one or more anatomic locations of the cancer of the subject. Numbered embodiment 180 comprises the computer system as in any of embodiments 153-179, wherein the predictive model is configured to receive the subject's biological sample cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof as an input to predict the cancer. Numbered embodiment 181 comprises the computer system as in any of embodiments 153-180, wherein detecting comprises whole genome sequencing, shotgun sequencing, targeted sequencing, RNA sequencing, methylation sequencing, or any combination thereof the one or more nucleic acid molecules of the biological sample. Numbered embodiment 182 comprises the computer system as in any of embodiments 153-181, wherein an area under a receiver operating curve of the predictive model for predicting the cancer of the subject is increased by at least 1%, at least 2%, at least 4%, at least 5%, or at least 10% when the combined decontaminated fungal presence and the decontaminated non-fungal presence is utilized during the correlation.

### EXAMPLES

### Example 1: Exploration of the Cancer Predictive Capabilities of Fungal Microbes

Fungal compositions, as described in the methods and systems herein, were acquired from multiple large cohorts of cancer samples, several of which were previously examined for bacterial compositions.

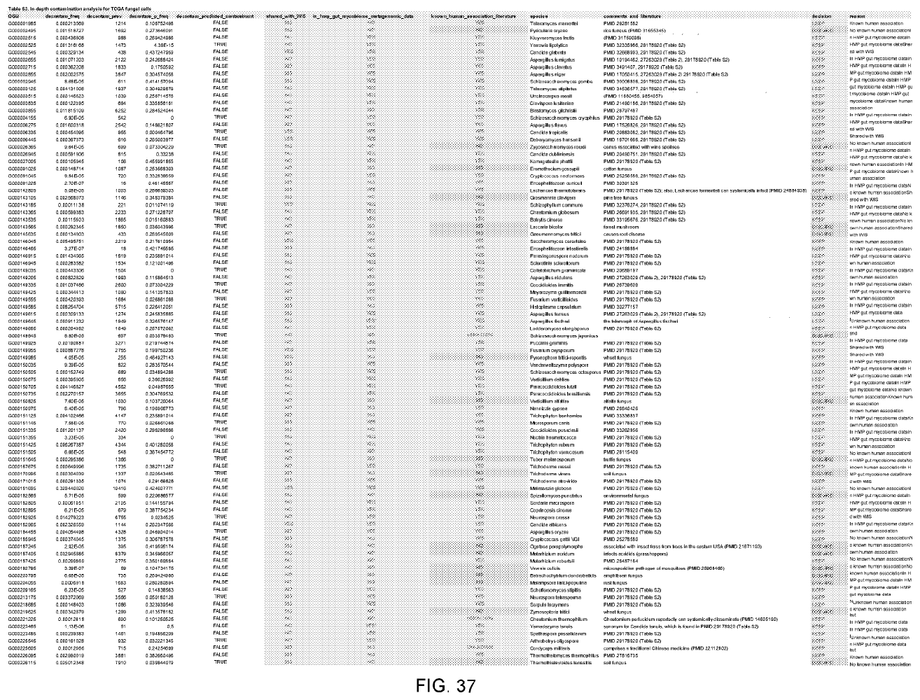

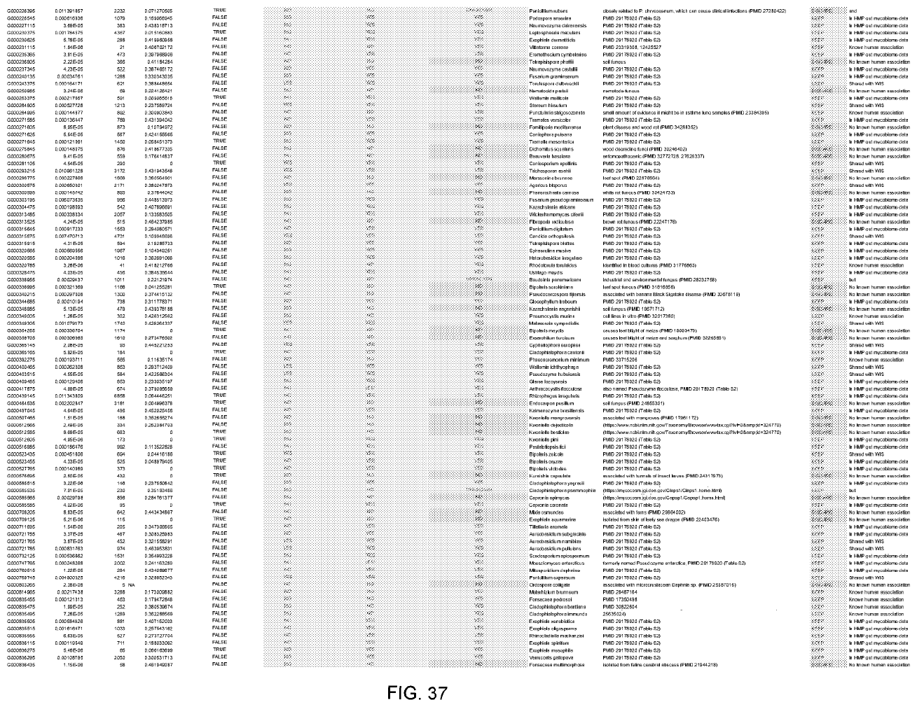

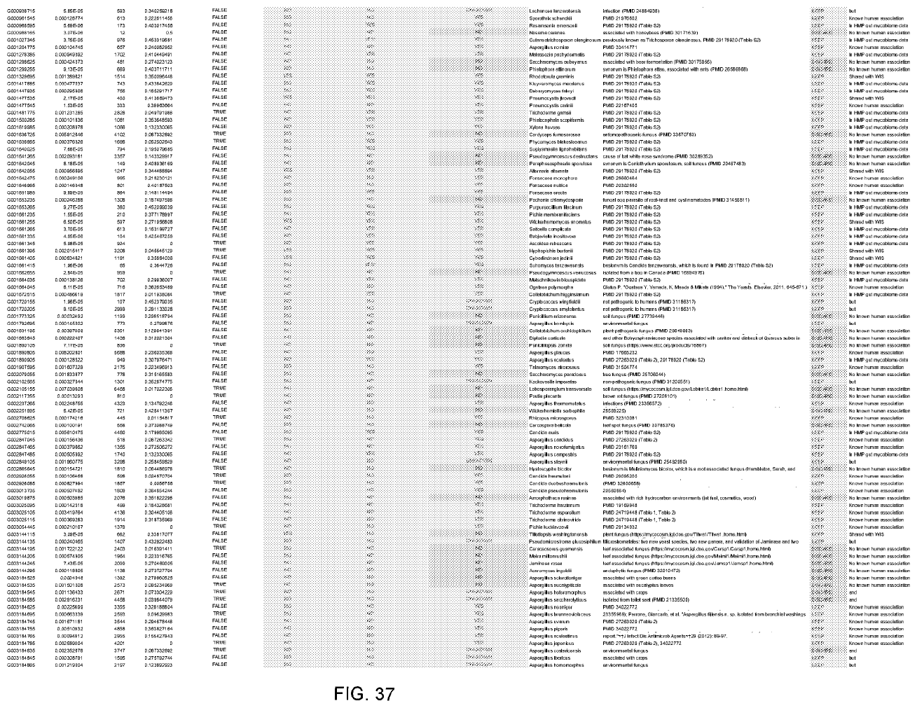

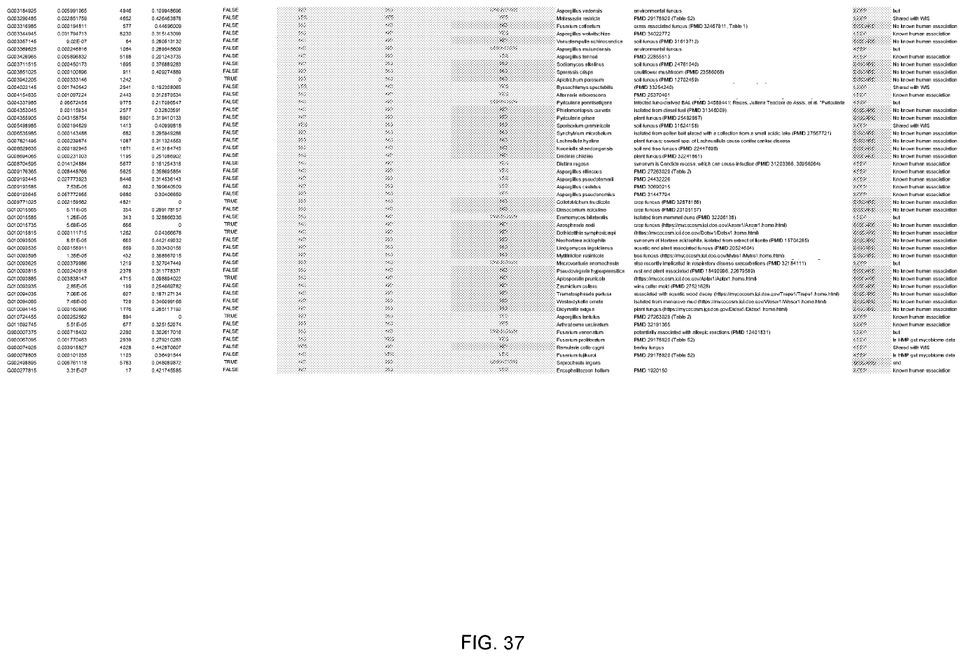

The first cohort encompassed whole-genome sequencing (WGS) and transcriptome sequencing (RNA-Seq) data from The Cancer Genome Atlas (TCGA). For quality control, all (˜10¹¹) unmapped DNA and RNA were re-aligned reads to a uniform human reference (GRCh38), removing poor-quality reads. Remaining reads were aligned to the RefSeq release 200 multi-domain database of 11,955 microbial (with 320 fungal) genomes. 15,512 samples (WGS: 4,736; RNA-Seq: 10,776) had non-zero microbial feature counts, of which, (97%) contained fungi. Of 6.06×10¹² total reads, 7.3% did not map to the human genome: 98.8% of these unmapped reads mapped to no organism in our microbial database. Of the remaining 1.2% of non-human reads that mapped to our microbial database (0.11% of total reads), 80.2% (0.067% of total) were classified as bacterial, and 2.3% (0.002% of total) as fungal, 1.172×10⁸ fungal reads for downstream analyses with an average read length of 57.4 bp SD=15.9; median=51 bp; a 45 bp minimum read length was enforced). Fungal-containing TCGA samples had an average of 7780 (95% CI: [7039, 8521]) fungal reads/sample. Although TCGA lacked contamination controls, in silico decontamination was implemented based on sequencing plate and center, and cross-referenced all fungal species against an independent cohort collected at the Weizmann Institute (WIS), the Human Microbiome Project (HMP)'s gut mycobiome cohort, and >100 other publications to obtain a final decontaminated list (FIG. 37). Of note, the cancer type abbreviations within TCGA and used elsewhere herein are shown in Table 1.

TABLE 1

| Study | |
|---|---|
| Abbreviation | Study Name |
| LAML | Acute Myeloid Leukemia |
| ACC | Adrenocortical carcinoma |
| BLCA | Bladder Urothelial Carcinoma |
| LGG | Brain Lower Grade Glioma |
| BRCA | Breast invasive carcinoma |
| CESC | Cervical squamous cell carcinoma and endocervical |
| | adenocarcinoma |
| CHOL | Cholangiocarcinoma |
| COAD | Colon adenocarcinoma |
| ESCA | Esophageal carcinoma |
| GBM | Glioblastoma multiforme |
| HNSC | Head and Neck squamous cell carcinoma |
| KICH | Kidney Chromophobe |
| KIRC | Kidney renal clear cell carcinoma |
| KIRP | Kidney renal papillary cell carcinoma |
| LIHC | Liver hepatocellular carcinoma |
| LUAD | Lung adenocarcinoma |
| LUSC | Lung squamous cell carcinoma |
| DLBC | Lymphoid Neoplasm Diffuse Large B-cell Lymphoma |
| MESO | Mesothelioma |
| OV | Ovarian serous cystadenocarcinoma |
| PAAD | Pancreatic adenocarcinoma |
| PCPG | Pheochromocytoma and Paraganglioma |
| PRAD | Prostate adenocarcinoma |
| READ | Rectum adenocarcinoma |
| SARC | Sarcoma |
| SKCM | Skin Cutaneous Melanoma |
| STAD | Stomach adenocarcinoma |
| TGCT | Testicular Germ Cell Tumors |
| THYM | Thymoma |
| THCA | Thyroid carcinoma |
| UCS | Uterine Carcinosarcoma |
| UCEC | Uterine Corpus Endometrial Carcinoma |
| UVM | Uveal Melanoma |

The second WIS cohort comprised independently collected tissue samples of tumor and normal adjacent tissue (NAT) from eight cancer types (bone, breast, colon, brain lung melanoma, ovary and pancreas). These samples underwent internal transcribed spacer 2 (ITS2) amplicon sequencing to characterize fungi and additionally had paraffin-only and DNA-extraction negative controls processed in parallel, which enabled removal of fungal contaminants.

The third cohort comprised more than four hundred plasma samples from treatment-naïve, early-stage, cancer-bearing patients across lung, pancreatic, colorectal, bile duct, gastric, ovarian, and breast cancers, as well as healthy individuals, that were independently collected and sequenced by a group at Johns Hopkins (PMID: 31142840). Raw sequencing data from these samples were extracted, human-depleted, and processed for fungal and non-fungal microbial presence and abundances.

The fourth cohort comprised more than hundred plasma samples from mostly treated, late-stage, cancer-bearing patients across prostate, lung, and melanoma cancers, as well as HIV-negative healthy individuals, that were formerly collected, sequenced, and analyzed for non-fungal microbial presence and abundances (PMID: 32214244). Raw sequencing data from these samples were extracted, human-depleted, and reprocessed to also identify fungal microbial presence and abundances in addition to non-fungal microbial presence and abundances.

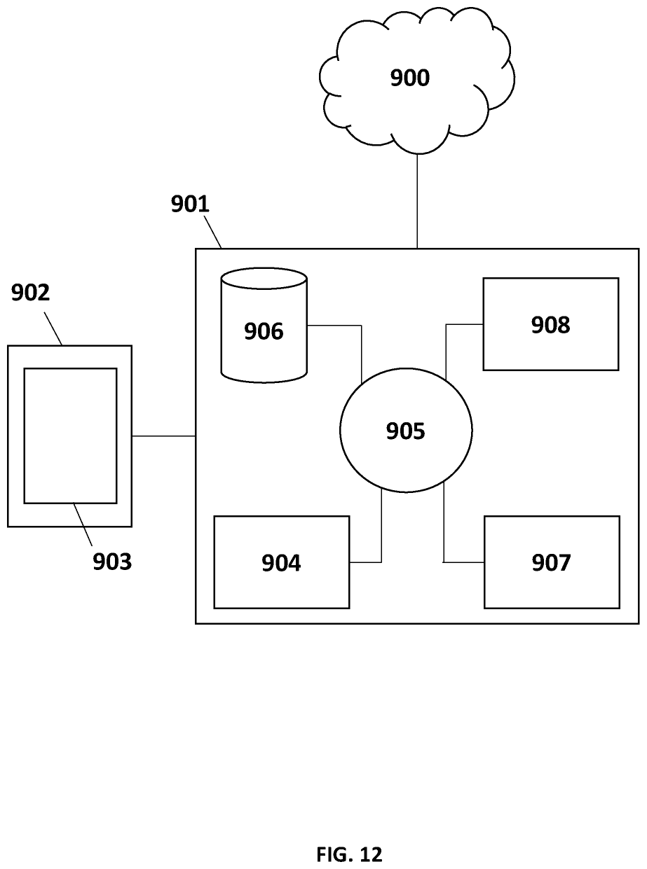

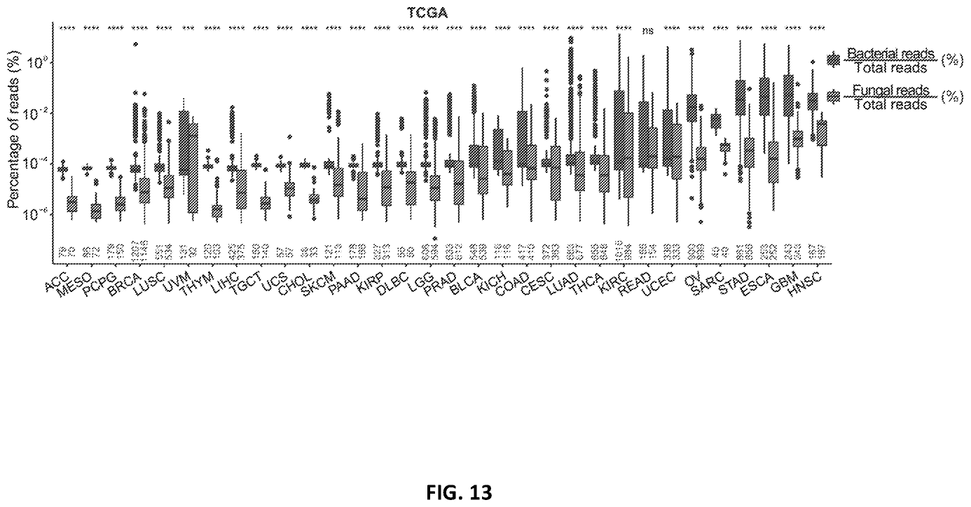

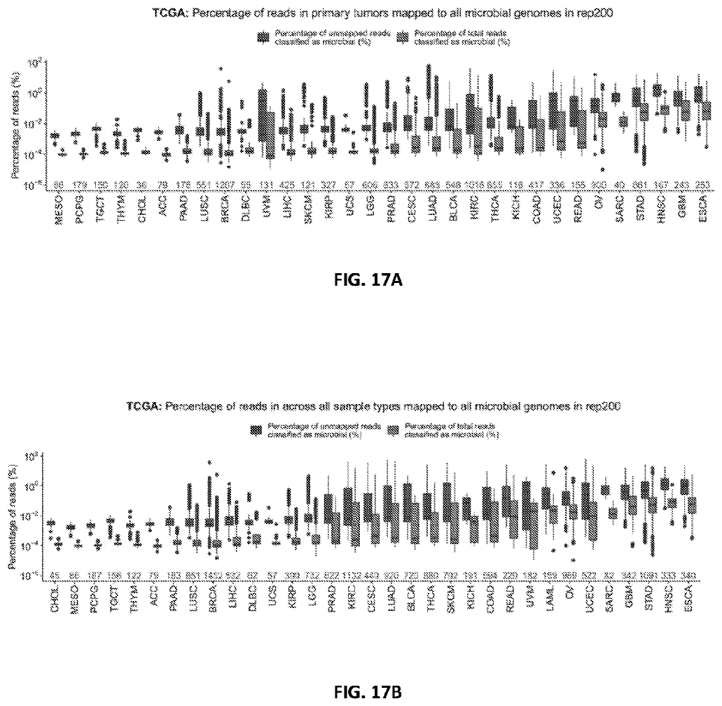

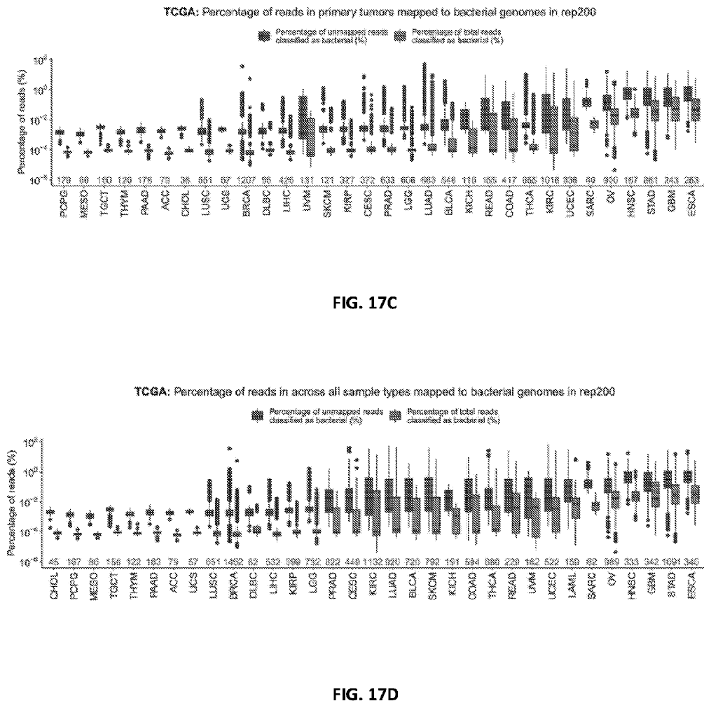

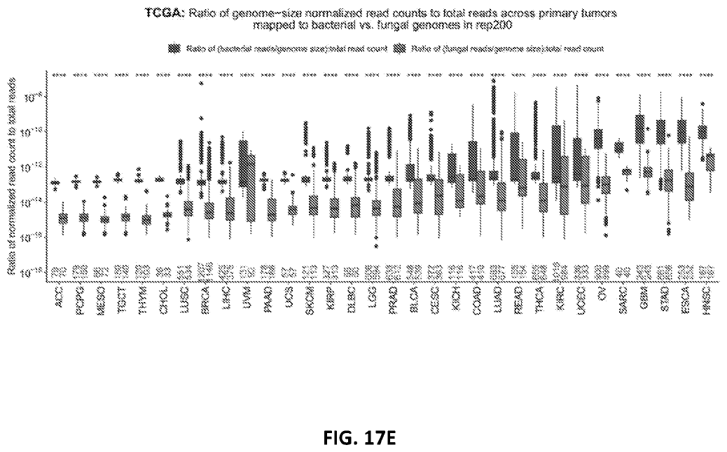

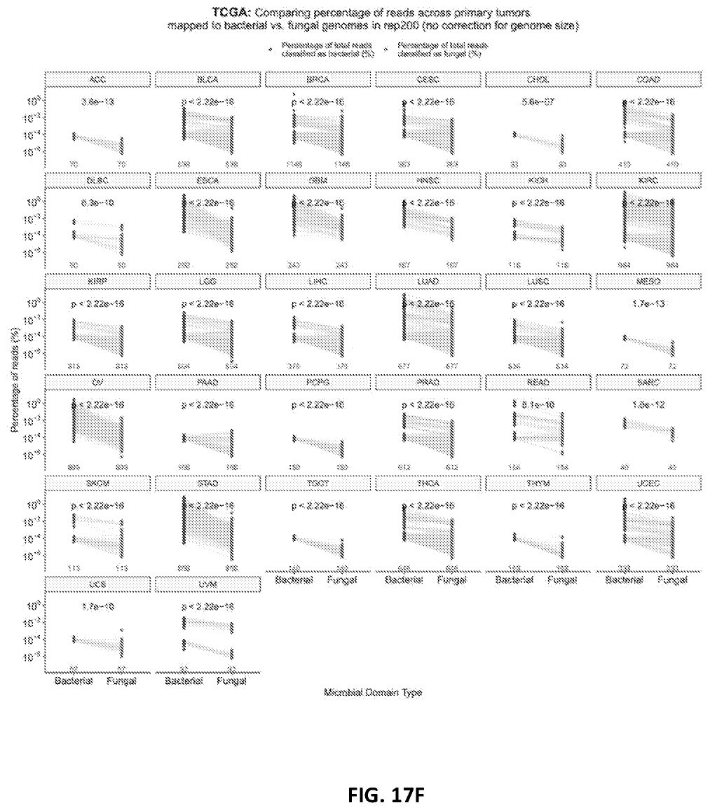

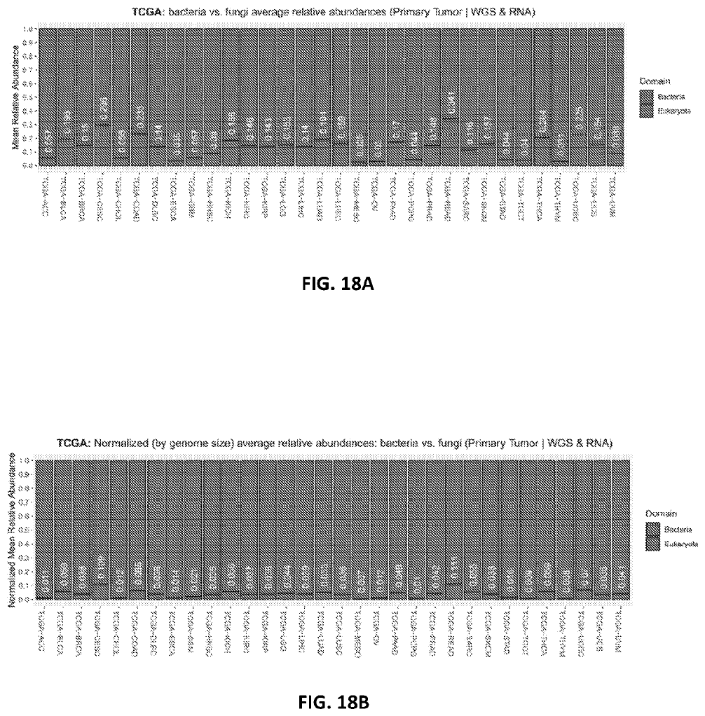

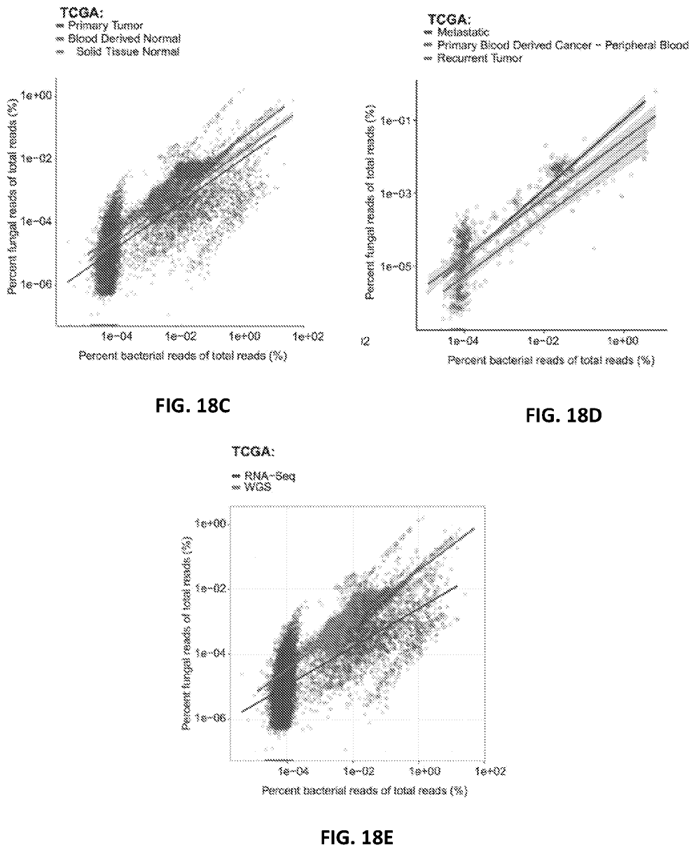

In the TCGA cohort, a significant, cancer type-specific differences in the percentage of classified fungal, bacterial, and pan-microbial reads out of total or unmapped reads was observed. In 31 of 32 cancer types, bacterial read proportions in primary tumors were significantly higher than fungal reads (FIG. 13), and all cancer types had significantly higher bacterial proportions during paired analyses (FIG. 17F) or after normalizing by genome sizes (FIG. 17E). Calculating average relative abundances of bacteria and fungi in TCGA primary tumors revealed 86.7% bacteria and 13.3% fungi without genome size normalization (FIG. 18A), or 96% bacteria and 4% fungi (FIG. 18B) with normalization, suggesting that bacteria predominate over fungi in the tumor microbiome. Fungal and bacterial read proportions had high Spearman correlations (FIG. 18C-E), including primary tumors (ρ=0.76, p<2.2×10⁻³⁰⁸), NATs (p=0.84, p<2.2×10⁻³⁰⁸), and blood (ρ=0.84, p<2.2×10⁻³⁰⁸). These data support a bacterial-dominated but polymicrobial cancer microbiome.

Motivated by the ˜117 million fungal reads in TCGA, per-sample and aggregate fungal genome coverages across all WGS and RNA-Seq samples (Table 2) were calculated. This revealed 31 fungi with ≥1% aggregate genome coverage, including Saccharomyces cerevisiae (99.7% overage), Malassezia restricta (98.6% coverage), Candida albicans (84.1% coverage), Malassezia globosa (40.5% coverage), and Blastomyces gilchristii (35.0% coverage). No one sample explained these top five aggregate coverages, ruling out the possibility that contamination solely explained them. Specifically, M restricta and globosa had no samples above 26.0% or 4.3% coverage, respectively. S. cerevisiae, C. albicans, and B. gilchristi had no samples above 64.8%, 50.0%, or 30.0% coverage, respectively. Many fungi had equally contributing coverages from different diseases and sequencing centers. Moreover, WIS-TCGA overlapping fungi were significantly more likely to have 10 aggregate genome coverage than non-WIS-overlapping species (Fisher exact test: p=0.05×10-8, odds ratio=13.). Several of these well-covered fungi were also identifiable when applying metagenomic assembly methods.

TABLE 2

| Pan-TCGA fungal genome coverages | | | | |
|---|---|---|---|---|
| OGU | covered_length | total_length | coverage_ratio | species |
| G000146045 | 12121399 | 12157425 | 0.997036708 | Saccharomyces cerevisiae |
| G003290485 | 7269088 | 7369807 | 0.986333563 | Malassezia restricta |
| G000182965 | 12021257 | 14282806 | 0.841659335 | Candida albicans |
| G000181695 | 3632704 | 8959438 | 0.405461146 | Malassezia globosa |
| G000003855 | 26409972 | 75406772 | 0.350233425 | Blastomyces gilchristii |
| G000226395 | 9758700 | 32224695 | 0.302832967 | Penicillium rubens |
| G002079055 | 3063377 | 12093003 | 0.253318138 | Saccharomyces paradoxus |
| G001642055 | 4111437 | 32992394 | 0.124617722 | Alternaria alternata |
| G004154835 | 3596204 | 33949929 | 0.105926702 | Alternaria arborescens |
| G000026945 | 1462577 | 14618562 | 0.100049307 | Candida dubliniensis |
| G000721785 | 2941007 | 29624778 | 0.099275242 | Aureobasidium pullulans |
| G000002545 | 1112432 | 12338568 | 0.090158923 | [Candida] glabrata |
| G001661405 | 958407 | 13019915 | 0.073610849 | Cyberlindnera jadinii |
| G000006335 | 693604 | 14630599 | 0.047407765 | Candida tropicalis |
| G000300575 | 1424553 | 30234305 | 0.047117108 | Agaricus bisporus |
| G004353045 | 1609976 | 40373728 | 0.039876823 | Phialemoniopsis curvata |
| G000349305 | 240871 | 7670969 | 0.031400335 | Malassezia sympodialis |
| G000006445 | 303690 | 12182088 | 0.024929224 | Debaryomyces hansenii |
| G000091025 | 193609 | 9119452 | 0.021230333 | Eremothecium gossypii |
| G001298625 | 234465 | 11734633 | 0.019980599 | Saccharomyces eubayanus |
| G900007375 | 761100 | 38660286 | 0.019686869 | Fusarium venenatum |
| G000769745 | 609280 | 32363668 | 0.018826049 | Penicillium expansum |
| G000149425 | 193544 | 10610114 | 0.018241463 | Meyerozyma guilliermondii |
| G000185945 | 257850 | 18375020 | 0.014032638 | Cryptococcus gattii VGI |
| G001417885 | 150197 | 10966627 | 0.013695825 | Kluyveromyces marxianus |
| G000243375 | 124605 | 9220818 | 0.013513443 | Torulaspora delbrueckii |
| G000002515 | 143173 | 10729567 | 0.013343782 | Kluyveromyces lactis |
| G000315645 | 345368 | 26054211 | 0.013255746 | Penicillium digitatum |
| G002775015 | 164394 | 12741558 | 0.01290219 | [Candida] auris |
| G000149645 | 409456 | 31778757 | 0.012884582 | Aspergillus fischeri |
| G000149955 | 630627 | 61388614 | 0.010272703 | Fusarium oxysporum |
| G000002525 | 196013 | 20551017 | 0.009537873 | Yarrowia lipolytica |
| G000003835 | 113082 | 12115052 | 0.009334009 | Clavispora lusitaniae |
| G003184545 | 327887 | 35611647 | 0.009207297 | Aspergillus heteromorphus |
| G900067095 | 410859 | 45210944 | 0.009087601 | Fusarium proliferatum |
| G000835515 | 345585 | 38227354 | 0.009040254 | Exophiala oligosperma |
| G000263375 | 78030 | 9816686 | 0.007948711 | Wallemia mellicola |
| G001447935 | 92352 | 11707957 | 0.007887969 | Debaryomyces fabryi |
| G902498895 | 166700 | 21189308 | 0.007867175 | Saprochaete ingens |
| G900079805 | 335986 | 43832534 | 0.00766522 | Fusarium fujikuroi |
| G001329695 | 147691 | 21014498 | 0.007028053 | Rhodotorula graminis |
| G001661255 | 98057 | 14146466 | 0.006931554 | Wickerhamomyces anomalus |
| G000149555 | 287457 | 41845334 | 0.006869511 | Fusarium verticillioides |
| G000146915 | 241267 | 37216127 | 0.006482862 | Parastagonospora nodorum |
| G001890805 | 178331 | 27994982 | 0.006370106 | Aspergillus glaucus |
| G000143535 | 269987 | 42630406 | 0.006333203 | Botrytis cinerea |
| G005498985 | 118595 | 19566268 | 0.006061197 | Sporisorium graminicola |
| G002849105 | 228395 | 37848680 | 0.006034424 | Aspergillus steynii |
| G000149685 | 92781 | 15547812 | 0.005967463 | Lodderomyces elongisporus |
| G000237345 | 66861 | 11219719 | 0.00595924 | Naumovozyma castellii |
| G001278385 | 48435 | 8152448 | 0.00594116 | Malassezia pachydermatis |
| G002251995 | 47941 | 8153160 | 0.005880051 | Wickerhamiella sorbophila |
| G003054445 | 63011 | 10812635 | 0.005827534 | Pichia kudriavzevii |
| G000171015 | 192453 | 36144224 | 0.005324585 | Trichoderma atroviride |
| G001672515 | 255215 | 50716583 | 0.005032181 | Colletotrichum higginsianum |
| G001641265 | 172296 | 35819841 | 0.004810072 | Pseudogymnoascus destructans |
| G000149585 | 156974 | 33035906 | 0.004751618 | Histoplasma capsulatum |
| G000150035 | 67574 | 14680191 | 0.004603074 | Vanderwaltozyma polyspora |
| G000167675 | 150625 | 33397233 | 0.004510104 | Trichoderma reesei |
| G000002715 | 119094 | 27862281 | 0.004274381 | Aspergillus clavatus |
| G002105155 | 180950 | 42771689 | 0.004230602 | Lobosporangium transversale |
| G000240135 | 152748 | 36458406 | 0.004189651 | Fusarium graminearum |
| G001664045 | 36641 | 8974491 | 0.004082794 | Ogataea polymorpha |
| G000227115 | 54981 | 13527780 | 0.004064303 | Naumovozyma dairenensis |
| G003316985 | 150948 | 37780712 | 0.003995372 | Fusarium coffeatum |
| G003184535 | 131963 | 34795012 | 0.003792584 | Aspergillus eucalypticola |
| G000315875 | 47765 | 12659541 | 0.003773044 | Candida orthopsilosis |
| G000026365 | 35716 | 9764755 | 0.003657644 | Zygosaccharomyces rouxii |
| G000315915 | 51223 | 14048773 | 0.003646084 | Tetrapisispora blattae |
| G000304475 | 40271 | 11130360 | 0.003618122 | Kazachstania africana |
| G002847485 | 100151 | 28258716 | 0.003544075 | Aspergillus campestris |
| G000264905 | 164316 | 46514783 | 0.003532554 | Stereum hirsutum |
| G000150735 | 105596 | 29953660 | 0.003525312 | Paracoccidioides brasiliensis |
| G002847465 | 110150 | 32441349 | 0.003395358 | Aspergillus novofumigatus |
| G000002855 | 110327 | 34007051 | 0.003244239 | Aspergillus niger |
| G001481775 | 121907 | 37911671 | 0.003215553 | Trichoderma gamsii |
| G000150705 | 101556 | 32927428 | 0.003084237 | Paracoccidioides lutzii |
| G001773325 | 102671 | 33736952 | 0.00304328 | Penicillium arizonense |
| G001661395 | 37245 | 12403630 | 0.00300275 | Hyphopichia burtonii |
| G000348985 | 32460 | 10846061 | 0.002992792 | Kazachstania naganishii |
| G000747765 | 52386 | 18115885 | 0.002891716 | Moesziomyces antarcticus |
| G000223485 | 37388 | 13182239 | 0.00283624 | Spathaspora passalidarum |
| G000142805 | 28900 | 10393002 | 0.002780717 | Lachancea thermotolerans |
| G000002655 | 81400 | 29385098 | 0.002770112 | Aspergillus fumigatus |
| G001661345 | 47707 | 17505238 | 0.002725299 | Ascoidea rubescens |
| G000003515 | 60699 | 22350618 | 0.002715764 | Uncinocarpus reesii |
| G000149985 | 100941 | 37998405 | 0.002656454 | Pyrenophora tritici-repentis |
| G003025115 | 86964 | 33225801 | 0.002617364 | Trichoderma citrinoviride |
| G000204055 | 263974 | 101138248 | 0.002610031 | Melampsora larici-populina |
| G000359705 | 109959 | 43021665 | 0.002555898 | Exserohilum turcicum |
| G003344945 | 94895 | 37519776 | 0.0025292 | Aspergillus welwitschiae |
| G002926085 | 31743 | 12580540 | 0.002523183 | [Candida] duobushaemulonis |
| G000523435 | 77705 | 31284796 | 0.002483794 | Bipolaris zeicola |
| G000236905 | 30000 | 12115390 | 0.002476189 | Tetrapisispora phaffii |
| G000182805 | 93655 | 38423023 | 0.002437471 | Sordaria macrospora |
| G000001985 | 69678 | 28652885 | 0.002431797 | Talaromyces marneffei |
| G000230375 | 109300 | 45126119 | 0.002422101 | Leptosphaeria maculans |
| G000354255 | 79762 | 32933287 | 0.002421926 | Bipolaris maydis |
| G000027005 | 22295 | 9216438 | 0.002419047 | Komagataella phaffii |
| G000235365 | 22843 | 9669564 | 0.002362361 | Eremothecium cymbalariae |
| G000523455 | 73762 | 31374457 | 0.002351021 | Bipolaris oryzae |
| G000721755 | 59597 | 25798196 | 0.002310123 | Aureobasidium subglaciale |
| G003025105 | 85877 | 37472970 | 0.002291705 | Trichoderma asperellum |
| G010094145 | 78815 | 34392727 | 0.002291618 | Didymella exigua |
| G000184455 | 86584 | 37912234 | 0.002283801 | Aspergillus oryzae |
| G000721765 | 57790 | 25429521 | 0.002272556 | Aureobasidium namibiae |
| G000006275 | 83184 | 36898944 | 0.002254373 | Aspergillus flavus |
| G000527765 | 73949 | 32843075 | 0.002251586 | Bipolaris victoriae |
| G000938715 | 24759 | 11092591 | 0.00223203 | Lachancea lanzarotensis |
| G003184835 | 81988 | 36949618 | 0.002218913 | Aspergillus costaricensis |
| G001640025 | 35300 | 15938318 | 0.002214788 | Sugiyamaella lignohabitans |
| G003184625 | 77566 | 35419804 | 0.002189905 | Aspergillus neoniger |
| G003184755 | 73696 | 35281251 | 0.002088815 | Aspergillus piperis |
| G004022145 | 61865 | 30107509 | 0.002054803 | Byssochlamys spectabilis |
| G000143365 | 71196 | 34887659 | 0.002040722 | Chaetomium globosum |
| G000143185 | 78479 | 38482726 | 0.002039331 | Schizophyllum commune |
| G008694065 | 77567 | 38655209 | 0.002006638 | Daldinia childiae |
| G004355905 | 89328 | 44558422 | 0.002004739 | Pyricularia grisea |
| G001653265 | 76652 | 38537841 | 0.001989006 | Purpureocillium lilacinum |
| G004337985 | 96640 | 49119180 | 0.00196746 | Pyricularia pennisetigena |
| G000182925 | 80786 | 41102778 | 0.001965463 | Neurospora crassa |
| G001477535 | 16325 | 8397620 | 0.001944003 | Pneumocystis jirovecii |
| G003184925 | 68964 | 35664554 | 0.001933685 | Aspergillus vadensis |
| G000281105 | 54852 | 28648681 | 0.001914643 | Coniosporium apollinis |
| G000303195 | 70456 | 36978859 | 0.001905305 | Fusarium pseudograminearum |
| G000149335 | 53191 | 29016139 | 0.001833152 | Coccidioides immitis |
| G000338995 | 62047 | 34412227 | 0.001803051 | Bipolaris sorokiniana |
| G003144135 | 30961 | 17184862 | 0.001801644 | Pseudomicrostroma glucosiphilum |
| G000151335 | 47960 | 27014492 | 0.001775343 | Coccidioides posadasii |
| G002102565 | 30540 | 17466453 | 0.001748495 | Kockovaella imperatae |
| G002237265 | 53950 | 30958894 | 0.001742633 | Aspergillus thermomutatus |
| G000149615 | 50608 | 29364542 | 0.001723439 | Aspergillus terreus |
| G010724455 | 51842 | 30244490 | 0.001714097 | Aspergillus lentulus |
| G010093625 | 56918 | 33337586 | 0.001707322 | Macroventuria anomochaeta |
| G000170995 | 66422 | 39024506 | 0.001702059 | Trichoderma virens |
| G003851025 | 65946 | 39020969 | 0.001690014 | Sparassis crispa |
| G009193645 | 60959 | 37787974 | 0.001613185 | Aspergillus pseudonomius |
| G000149845 | 18764 | 11733774 | 0.001599144 | Schizosaccharomyces japonicus |
| G002926055 | 21163 | 13314543 | 0.001589465 | [Candida] haemuloni |
| G000150825 | 51381 | 32863508 | 0.001563467 | Verticillium alfalfae |
| G000320565 | 45530 | 29353523 | 0.001551091 | Sphaerulina musiva |
| G000708205 | 20832 | 13637588 | 0.001527543 | Mixia osmundae |
| G001661415 | 19525 | 13136504 | 0.001486316 | Suhomyces tanzawaensis |
| G003942205 | 37606 | 25480076 | 0.001475898 | Apiotrichum porosum |
| G000219625 | 58405 | 39686651 | 0.001471654 | Zymoseptoria tritici |
| G000209165 | 22671 | 15441319 | 0.001468204 | Scheffersomyces stipitis |
| G000149035 | 75004 | 51657367 | 0.001451952 | Colletotrichum graminicola |
| G000146945 | 55581 | 38459884 | 0.001445168 | Sclerotinia sclerotiorum |
| G000151425 | 32111 | 22530713 | 0.00142521 | Trichophyton rubrum |
| G001636725 | 47583 | 33494542 | 0.00142062 | Cordyceps fumosorosea |
| G000226115 | 52408 | 36912356 | 0.001419796 | Thermothielavioides terrestris |
| G000151145 | 32818 | 23263411 | 0.001410713 | Microsporum canis |
| G009193445 | 53760 | 38248889 | 0.001405531 | Aspergillus pseudotamarii |
| G001831195 | 67682 | 48562862 | 0.001393699 | Colletotrichum orchidophilum |
| G000149925 | 123510 | 88732216 | 0.001391941 | Puccinia graminis |
| G001638985 | 74973 | 53940747 | 0.001389914 | Phycomyces blakesleeanus |
| G000003125 | 49476 | 35701823 | 0.001385812 | Talaromyces stipitatus |
| G008704595 | 18471 | 13449319 | 0.001373378 | Diutina rugosa |
| G000091045 | 26132 | 19052182 | 0.001371601 | Cryptococcus neoformans |
| G000225605 | 43735 | 32269198 | 0.001355317 | Cordyceps militaris |
| G000151125 | 29843 | 22225500 | 0.001342737 | Trichophyton benhamiae |
| G000002945 | 16777 | 12591311 | 0.001332427 | Schizosaccharomyces pombe |
| G000230625 | 34769 | 26376967 | 0.001318158 | Exophiala dermatitidis |
| G003144115 | 24169 | 18762681 | 0.001288142 | Tilletiopsis washingtonensis |
| G003184525 | 47146 | 36723105 | 0.001283824 | Aspergillus sclerotioniger |
| G000150505 | 14900 | 11634553 | 0.001280668 | Schizosaccharomyces octosporus |
| G000149205 | 38587 | 30277769 | 0.001274433 | Aspergillus nidulans |
| G000226095 | 49315 | 38744336 | 0.001272831 | Thermothelomyces thermophilus |
| G001661235 | 14564 | 11582330 | 0.001257433 | Pichia membranifaciens |
| G003025095 | 51114 | 40991268 | 0.001246948 | Trichoderma harzianum |
| G000213175 | 48224 | 39147933 | 0.00123184 | Neurospora tetrasperma |
| G000271585 | 54813 | 44799648 | 0.001223514 | Trametes versicolor |
| G000203795 | 29701 | 24317601 | 0.001221379 | Batrachochytrium dendrobatidis |
| G000280675 | 40572 | 33702514 | 0.001203827 | Beauveria bassiana |
| G000150975 | 27572 | 23273302 | 0.001184705 | Nannizzia gypsea |
| G000497045 | 20248 | 17326287 | 0.001168629 | Kalmanozyma brasiliensis |
| G010093885 | 38292 | 32825345 | 0.001166538 | Aplosporella prunicola |
| G000277815 | 2626 | 2252004 | 0.001166073 | Encephalitozoon hellem |
| G000313485 | 18523 | 15908412 | 0.001164353 | Wickerhamomyces ciferrii |
| G001890105 | 30151 | 26093131 | 0.001155515 | Penicilliopsis zonata |
| G003144205 | 20078 | 17384135 | 0.001154961 | Meira miltonrushii |
| G001890905 | 40131 | 35437594 | 0.001132441 | Aspergillus aculeatus |
| G000143105 | 32996 | 29799766 | 0.001107257 | Grosmannia clavigera |
| G000403515 | 20312 | 18444398 | 0.001101256 | Pseudozyma hubeiensis |
| G001883845 | 38483 | 34989679 | 0.001099839 | Diplodia corticola |
| G002742065 | 39708 | 37062053 | 0.001071392 | Cercospora beticola |
| G000146465 | 2374 | 2217098 | 0.001070769 | Encephalitozoon intestinalis |
| G000814965 | 39664 | 37067986 | 0.001070034 | Metarhizium brunneum |
| G003144295 | 20652 | 19330651 | 0.001068355 | Acaromyces ingoldii |
| G000187245 | 9386 | 8874709 | 0.001057612 | Ogataea parapolymorpha |
| G001477545 | 7896 | 7662676 | 0.001030449 | Pneumocystis carinii |
| G010015815 | 35129 | 34434679 | 0.001020163 | Dothidotthia symphoricarpi |
| G003426965 | 38653 | 38719648 | 0.000998279 | Aspergillus tanneri |
| G011692745 | 23434 | 23563336 | 0.000994511 | Arthroderma uncinatum |
| G000298775 | 51635 | 51951637 | 0.000993905 | Marssonina brunnea |
| G003013735 | 12511 | 12591975 | 0.000993569 | [Candida] pseudohaemulonis |
| G000293215 | 24261 | 24541851 | 0.000988556 | Trichosporon asahii |
| G000223465 | 10520 | 10747530 | 0.00097883 | Yamadazyma tenuis |
| G000143565 | 62916 | 64890724 | 0.000969568 | Laccaria bicolor |
| G000516985 | 50116 | 51911486 | 0.000965413 | Pestalotiopsis fici |
| G001792695 | 35897 | 37483585 | 0.000957673 | Aspergillus bombycis |
| G003711515 | 41188 | 43450656 | 0.000947926 | Sodiomyces alkalinus |
| G001204775 | 33963 | 36159580 | 0.000939253 | Aspergillus nomiae |
| G000218685 | 40161 | 42798133 | 0.000938382 | Serpula lacrymans |
| G003184585 | 29069 | 31098569 | 0.000934738 | Aspergillus saccharolyticus |
| G000732125 | 39714 | 43440639 | 0.000914213 | Scedosporium apiospermum |
| G000150675 | 30909 | 33901404 | 0.000911732 | Verticillium dahliae |
| G000400465 | 8751 | 9657252 | 0.000906158 | Wallemia ichthyophaga |
| G001664035 | 14403 | 16056143 | 0.00089704 | Metschnikowia bicuspidata |
| G003184785 | 32234 | 36103194 | 0.00089283 | Aspergillus japonicus |
| G003144195 | 21398 | 24153100 | 0.000885932 | Ceraceosorus guamensis |
| G000226545 | 30064 | 34719534 | 0.00086591 | Podospora anserina |
| G000836115 | 28073 | 32912889 | 0.000852949 | Exophiala spinifera |
| G000221225 | 24052 | 28323206 | 0.000849198 | Chaetomium thermophilum |
| G009193585 | 33961 | 40030911 | 0.000848369 | Aspergillus caelatus |
| G000187405 | 33436 | 39427129 | 0.000848046 | Metarhizium acridum |
| G000349005 | 6228 | 7501723 | 0.000830209 | Pneumocystis murina |
| G009771025 | 48172 | 58065355 | 0.000829617 | Colletotrichum fructicola |
| G000328475 | 16102 | 19664876 | 0.00081882 | Ustilago maydis |
| G003184745 | 28940 | 35854123 | 0.00080716 | Aspergillus uvarum |
| G000760515 | 4315 | 5647252 | 0.000764088 | Mitosporidium daphniae |
| G008629635 | 16059 | 21300174 | 0.000753938 | Kwoniella shandongensis |
| G000464535 | 27896 | 37148751 | 0.000750927 | Endocarpon pusillum |
| G003184765 | 27359 | 36474049 | 0.000750095 | Aspergillus aculeatinus |
| G010093815 | 26272 | 35442567 | 0.000741256 | Pseudovirgaria hyperparasitica |
| G000002495 | 30356 | 40980161 | 0.000740749 | Pyricularia oryzae |
| G000271645 | 20769 | 28640799 | 0.000725154 | Tremella mesenterica |
| G000151355 | 37167 | 51290657 | 0.000724635 | [Nectria] haematococca |
| G000585565 | 20588 | 28889978 | 0.000712635 | Capronia epimyces |
| G002847045 | 19141 | 27323112 | 0.000700542 | Aspergillus candidus |
| G000417875 | 16204 | 23306140 | 0.000695267 | Anthracocystis flocculosa |
| G000961545 | 22432 | 32375403 | 0.000692872 | Sporothrix schenckii |
| G009176365 | 27786 | 40175128 | 0.000691622 | Aspergillus alliaceus |
| G003184695 | 25663 | 37480240 | 0.000684707 | Aspergillus brunneoviolaceus |
| G003184865 | 23271 | 34057886 | 0.000683278 | Aspergillus homomorphus |
| G001027345 | 13553 | 19839138 | 0.000683145 | Cutaneotrichosporon oleaginosum |
| G001661265 | 9546 | 14136866 | 0.000675256 | Saitoella complicata |
| G000576695 | 7671 | 11371366 | 0.000674589 | Kuraishia capsulata |
| G001651985 | 23096 | 34749095 | 0.00066465 | Fonsecaea erecta |
| G000145635 | 28606 | 43778984 | 0.000653419 | Gaeumannomyces tritici |
| G000835505 | 20458 | 31406040 | 0.000651403 | Exophiala xenobiotica |
| G003184845 | 21772 | 33440283 | 0.000651071 | Aspergillus ibericus |
| G000182565 | 15542 | 24131852 | 0.000644045 | Spizellomyces punctatus |
| G000004155 | 7365 | 11555344 | 0.000637367 | Schizosaccharomyces cryophilus |
| G000836295 | 20231 | 31787789 | 0.000636439 | Verruconis gallopava |
| G001907595 | 19114 | 30859502 | 0.000619388 | Talaromyces atroroseus |
| G000151505 | 13946 | 22551407 | 0.000618409 | Trichophyton verrucosum |
| G000320585 | 20276 | 33650247 | 0.000602551 | Heterobasidion irregulare |
| G001619985 | 14375 | 24338283 | 0.000590633 | Xylona heveae |
| G001642475 | 20324 | 35236257 | 0.000576792 | Fonsecaea monophora |
| G000439145 | 75430 | 136748513 | 0.000551596 | Rhizophagus irregularis |
| G001646965 | 18342 | 33792587 | 0.000542782 | Fonsecaea nubica |
| G010015735 | 21086 | 38902129 | 0.000542027 | Aaosphaeria arxii |
| G000091225 | 1304 | 2497719 | 0.000522076 | Encephalitozoon cuniculi |
| G007821495 | 17503 | 33842024 | 0.000517197 | Lachnellula hyalina |
| G001653235 | 22855 | 44216763 | 0.000516885 | Pochonia chlamydosporia |
| G000338955 | 11005 | 21875980 | 0.000503063 | Baudoinia panamericana |
| G000365145 | 14367 | 28781663 | 0.000499172 | Cyphellophora europaea |
| G000968595 | 13872 | 28266025 | 0.000490766 | Rasamsonia emersonii |
| G003369625 | 21545 | 45345076 | 0.000475134 | Aspergillus mulundensis |
| G010015565 | 12559 | 26538295 | 0.000473241 | Dissoconium aciculare |
| G003019875 | 13502 | 28633538 | 0.000471545 | Amorphotheca resinae |
| G010093505 | 9587 | 20433208 | 0.000469187 | Neohortaea acidophila |
| G001299255 | 14126 | 30366235 | 0.000465188 | Phialophora attinorum |
| G000225545 | 18129 | 40077109 | 0.000452353 | Arthrobotrys oligospora |
| G000187425 | 18671 | 41658580 | 0.000448191 | Metarhizium robertsii |
| G002865645 | 36513 | 82388947 | 0.000443178 | Hyaloscypha bicolor |
| G002708625 | 11277 | 25974995 | 0.000434148 | Rhizopus microsporus |
| G001661335 | 6606 | 15222010 | 0.000433977 | Babjeviella inositovora |
| G001642045 | 16130 | 38465224 | 0.00041934 | Paraphaeosphaeria sporulosa |
| G000507465 | 8769 | 22655731 | 0.000387054 | Kwoniella mangrovensis |
| G000392275 | 18165 | 47477909 | 0.000382599 | Phaeoacremonium minimum |
| G010094085 | 10292 | 26981606 | 0.000381445 | Westerdykella ornata |
| G900074925 | 11990 | 32256462 | 0.000371708 | Ramularia collo-cygni |
| G000320785 | 7432 | 20225802 | 0.000367451 | Rhodotorula toruloides |
| G000182895 | 12444 | 36193930 | 0.000343815 | Coprinopsis cinerea |
| G000365165 | 9291 | 28991471 | 0.000320474 | Cladophialophora carrionii |
| G000835455 | 10662 | 34689080 | 0.000307359 | Fonsecaea pedrosoi |
| G000340215 | 22399 | 74142267 | 0.000302108 | Pseudocercospora fijiensis |
| G001662655 | 9082 | 30177856 | 0.000300949 | Pseudogymnoascus verrucosus |
| G002117355 | 12715 | 42463848 | 0.000299431 | Postia placenta |
| G000271605 | 17801 | 63382639 | 0.00028085 | Fomitiporia mediterranea |
| G000585535 | 10728 | 39422567 | 0.000272128 | Cladophialophora psammophila |
| G000151645 | 33874 | 124953642 | 0.000271093 | Tuber melanosporum |
| G000344685 | 9722 | 37187045 | 0.000261435 | Gloeophyllum trabeum |
| G000264995 | 8884 | 34175781 | 0.00025995 | Punctularia strigosozonata |
| G010094035 | 12241 | 47747773 | 0.000256368 | Trematosphaeria pertusa |
| G010093935 | 9376 | 38253023 | 0.000245105 | Zasmidium cellare |
| G000835475 | 8780 | 36723732 | 0.000239082 | Cladophialophora bantiana |
| G000313525 | 6606 | 28395239 | 0.000232645 | Fibroporia radiculosa |
| G000512565 | 5459 | 23862632 | 0.000228768 | Kwoniella dejecticola |
| G000836275 | 6508 | 29273883 | 0.000222314 | Exophiala mesophila |
| G000300595 | 10244 | 46316045 | 0.000221176 | Phanerochaete carnosa |
| G000709125 | 9052 | 41569958 | 0.000217753 | Exophiala aquamarina |
| G000275845 | 9134 | 42759250 | 0.000213615 | Dichomitus squalens |
| G000835495 | 8878 | 43034672 | 0.000206299 | Cladophialophora immunda |
| G010093535 | 13853 | 69034626 | 0.000200667 | Lindgomyces ingoldianus |
| G001500285 | 9594 | 48877657 | 0.000196286 | Phialocephala scopiformis |
| G000585585 | 4971 | 25807813 | 0.000192616 | Capronia coronata |
| G000409485 | 7562 | 39510154 | 0.000191394 | Glarea lozoyensis |
| G003144245 | 3035 | 16615986 | 0.000182655 | Jaminaea rosea |
| G010015585 | 4899 | 26865895 | 0.00018235 | Eremomyces bilateralis |
| G010093595 | 8395 | 47276492 | 0.000177572 | Mytilinidion resinicola |
| G001720205 | 3575 | 20255276 | 0.000176497 | Cryptococcus amylolentus |
| G000585515 | 4823 | 27898562 | 0.000172876 | Cladophialophora yegresii |
| G000711695 | 3231 | 18709770 | 0.000172691 | Tilletiaria anomala |
| G000271625 | 7384 | 42972724 | 0.00017183 | Coniophora puteana |
| G006535985 | 4409 | 26247535 | 0.000167978 | Synchytrium microbalum |
| G000512585 | 3770 | 24360992 | 0.000154756 | Kwoniella bestiolae |
| G003357145 | 5082 | 33640415 | 0.000151068 | Venustampulla echinocandica |
| G000836435 | 4912 | 33448395 | 0.000146853 | Fonsecaea multimorphosa |
| G001720155 | 2846 | 19971439 | 0.000142504 | Cryptococcus wingfieldii |
| G000512605 | 2942 | 20828924 | 0.000141246 | Kwoniella pini |
| G000803265 | 276 | 2290808 | 0.000120482 | Ordospora colligata |
| G000988165 | 642 | 5701448 | 0.000112603 | Nosema ceranae |
| G000231115 | 325 | 3217896 | 0.000100998 | Vittaforma corneae |
| G000250985 | 383 | 4072626 | 9.40E−05 | Nematocida parisii |
| G000192795 | 573 | 6126249 | 9.35E−05 | Vavraia culicis |
| G000835555 | 2184 | 32474751 | 6.73E−05 | Rhinocladiella mackenziei |

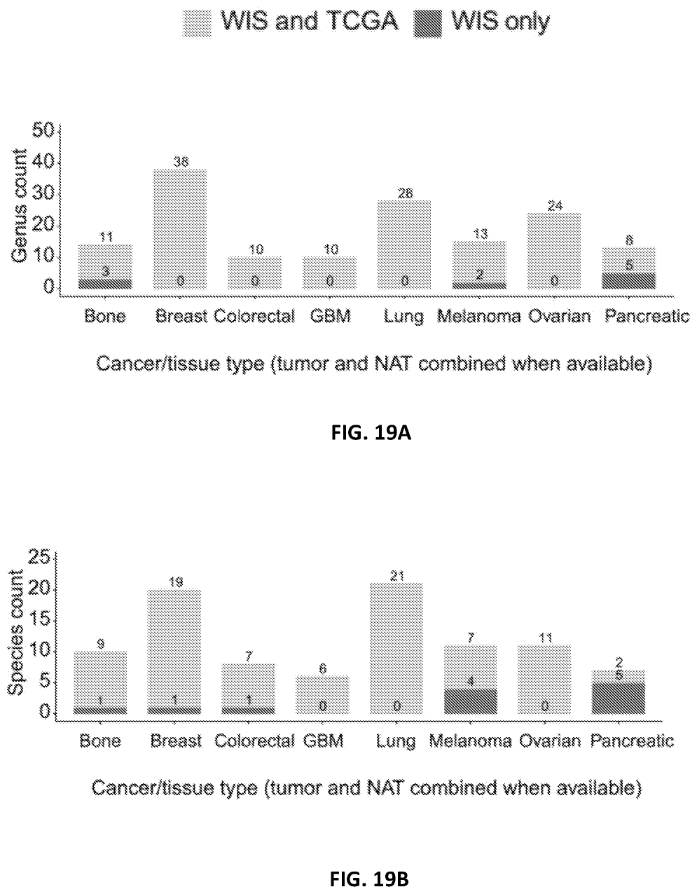

Despite geographical and technical processing differences between the TCGA and WIS samples, it was identified, within the intersection of the WIS cohort and TCGA fungal reference database, that 87.2% of WIS species- and 93.4% of fungal genera existed in matched TCGA cancer types (FIGS. 19A-19B). To be conservative, versions of TCGA mycobiome data subset to WIS-intersecting fungi, with similar conclusions were then analyzed in downstream machine learning analyses irrespective of cohort.

### Different Cancer Types Exhibit Cancer Type-Specific Mycobiomes

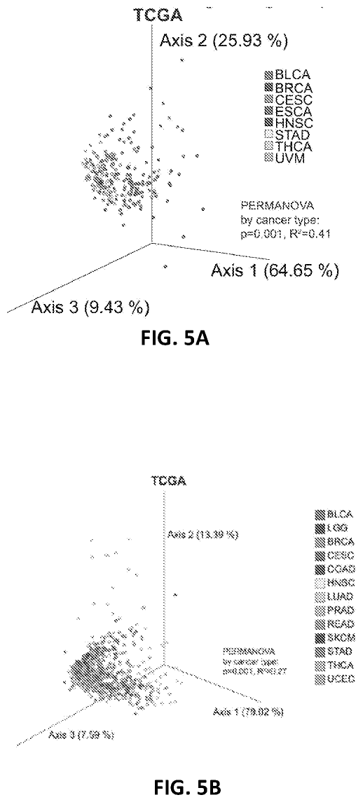

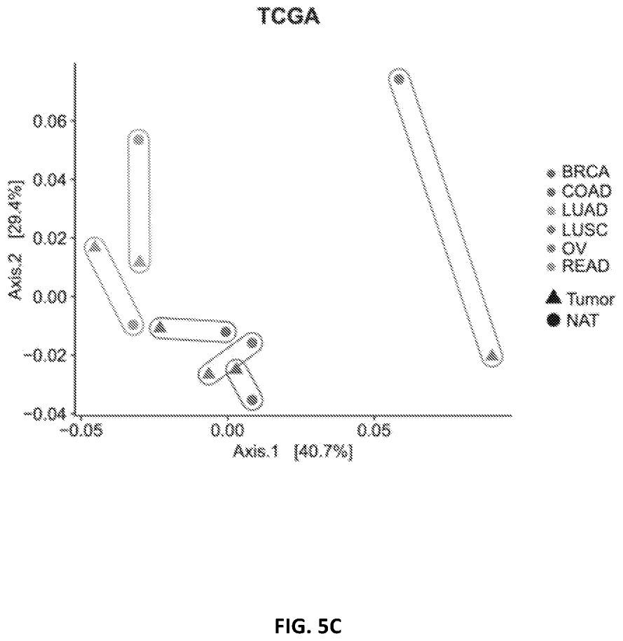

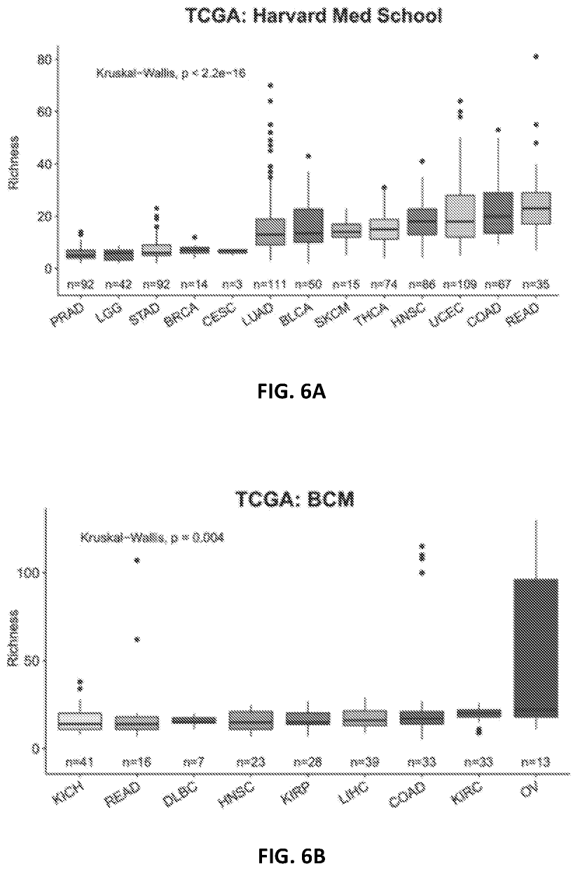

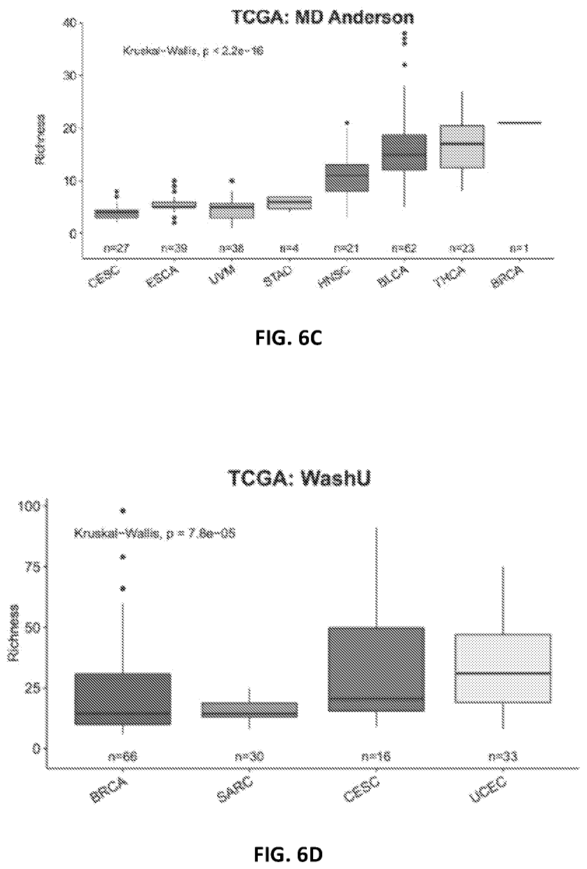

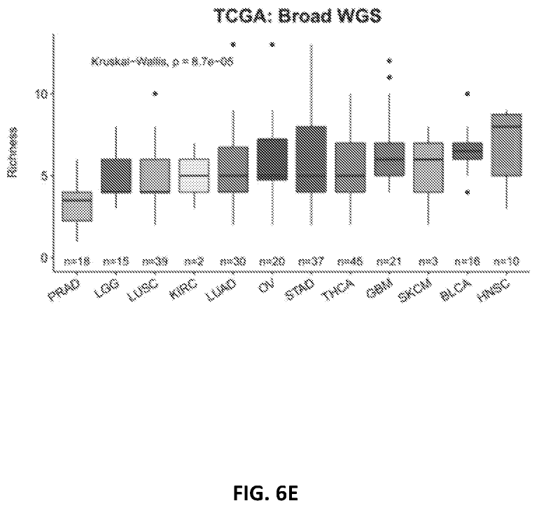

Tumor mycobiome richness varied significantly across TCGA cancer types (FIGS. 6A-6E). Similarity, beta diversity analyses within TCGA sequencing centers revealed cancer-type specific mycobiome compositions (FIG. 5A-5B). Interestingly, the TCGA cohort demonstrated co-clustering of tumor and NAT samples when comparing beta-diversity scores, supporting similar tumor and NAT compositions (FIG. 5C). Collectively, these analyses portray ubiquitous, low-abundance, cancer type-specific mycobiomes that have community assemblies similar to those in adjacent normal tissues.

### Intratumoral Mycobiome-Bacteriome-Immunome Interactions

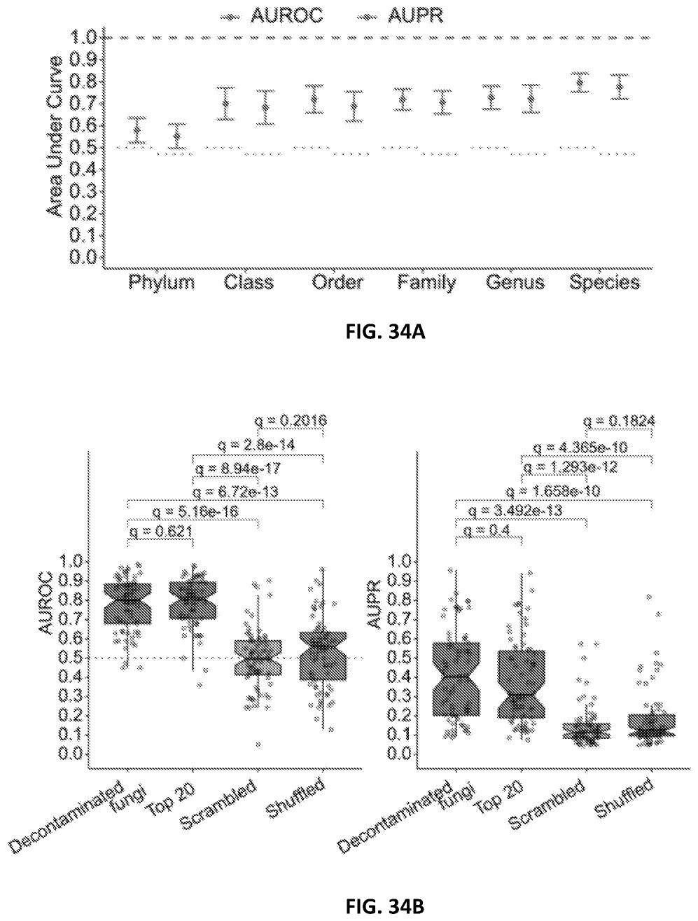

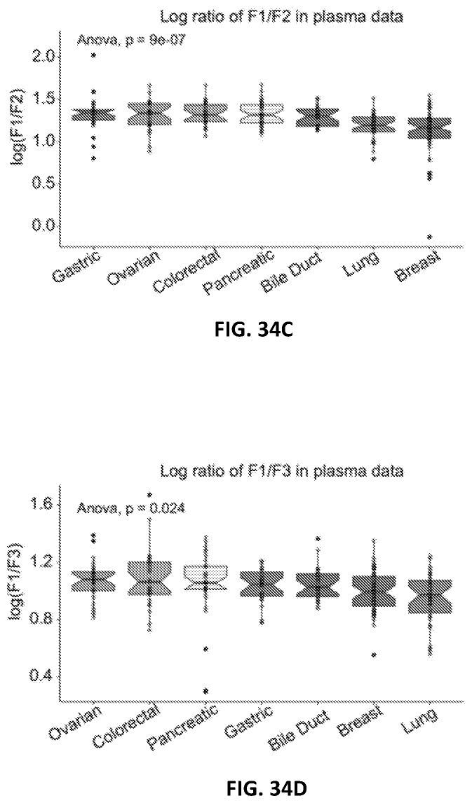

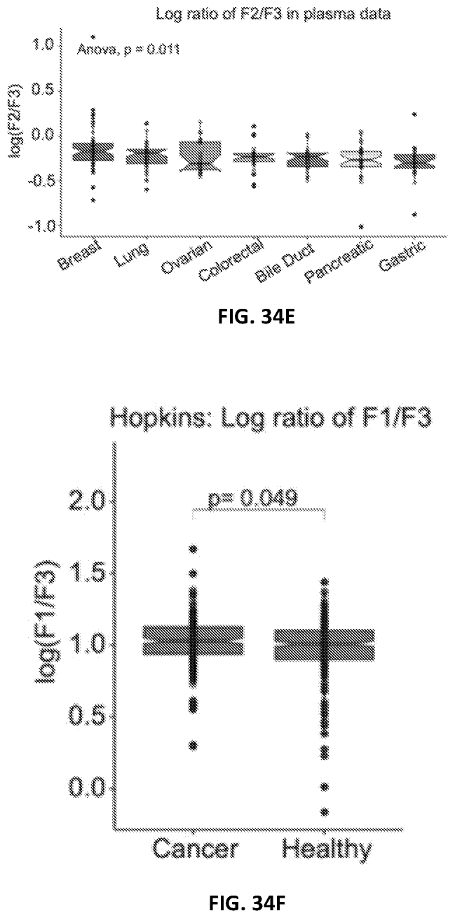

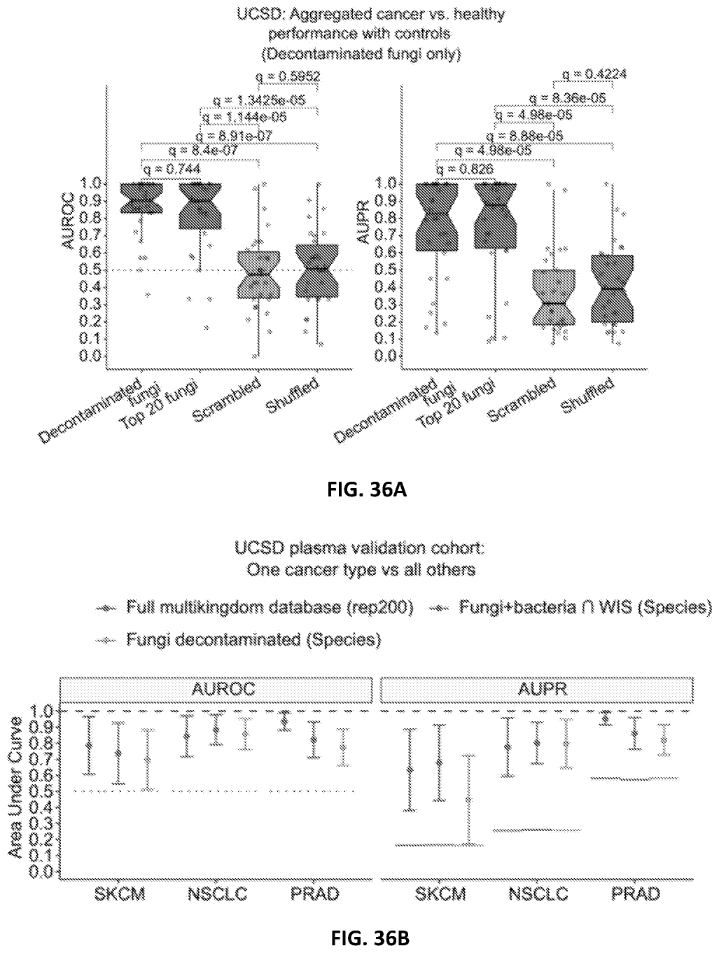

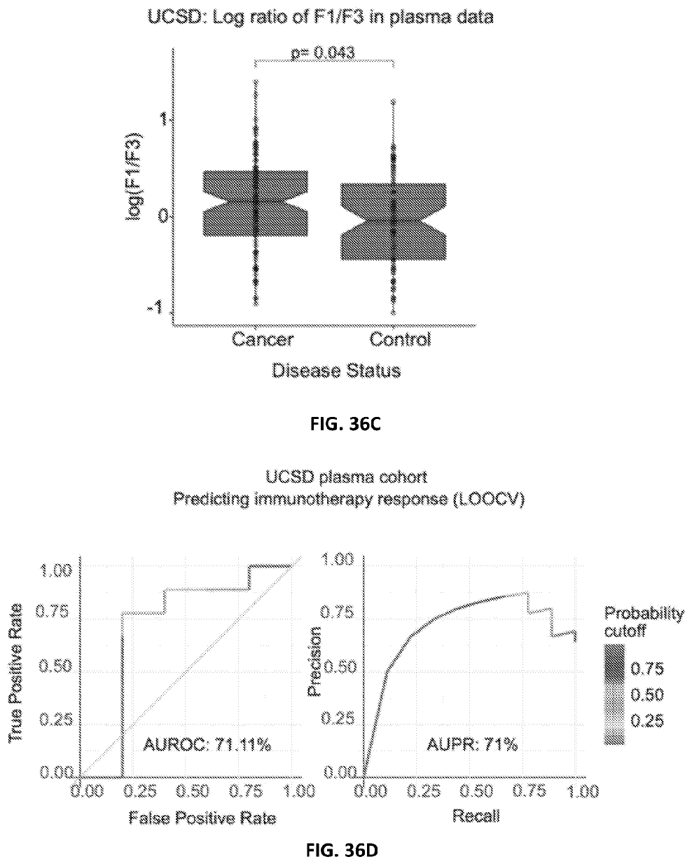

Fungi interact with bacteria by physical and biochemical mechanisms, as well as with host immune cells, motivating exploration of inter-domain connections between mycobiome, bacteriome, and immunome data in TCGA. These were correlated using WIS-overlapping fungal and bacterial genera in TCGA alongside CIBERSORT-derived immune cell compositions (PMID: 29628290) using a tool called MMvec (PMID: 31686038). Clustering of the data revealed groups of bacteria and immune cells co-occurring with specific types of fungi, herein termed “mycotypes,” which were used to calculate log-ratios of microbial abundances, which varied across cancer types in multiple cohorts, including in plasma-derived mycobiomes across several cancer types (FIGS. 34C-34E) and cancer versus healthy comparisons (FIGS. 34F, 36C).

### Statistical and Machine Learning Analysis Demonstrate Cancer-Type Specific Mycobiomes

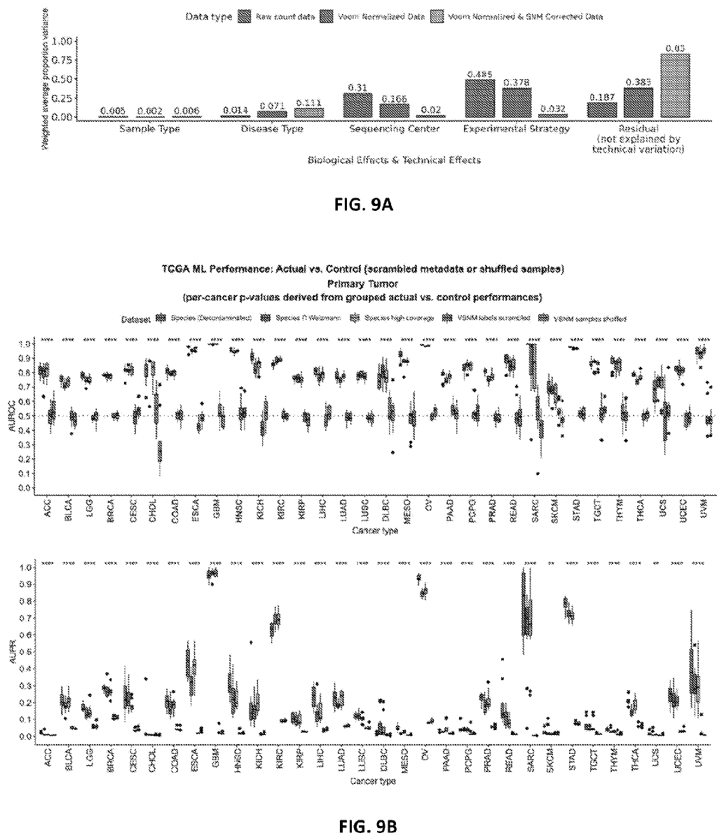

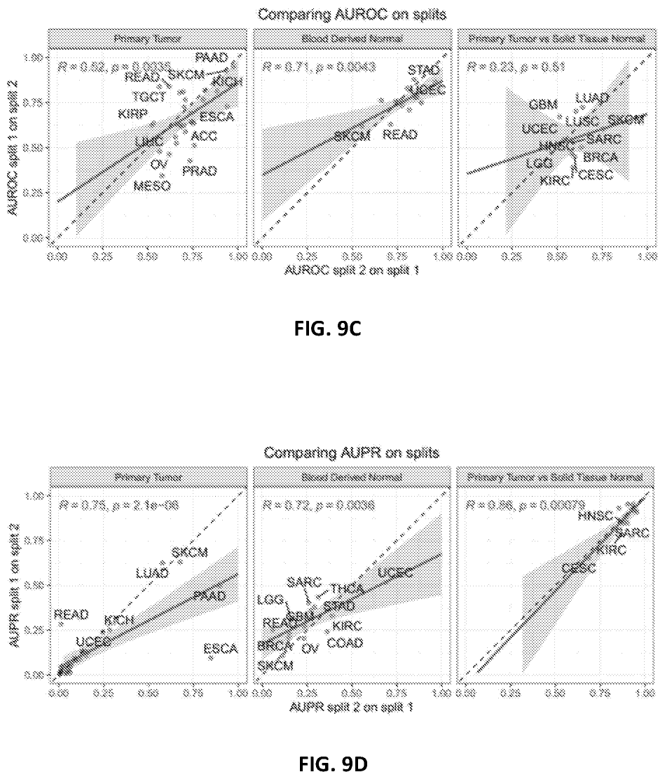

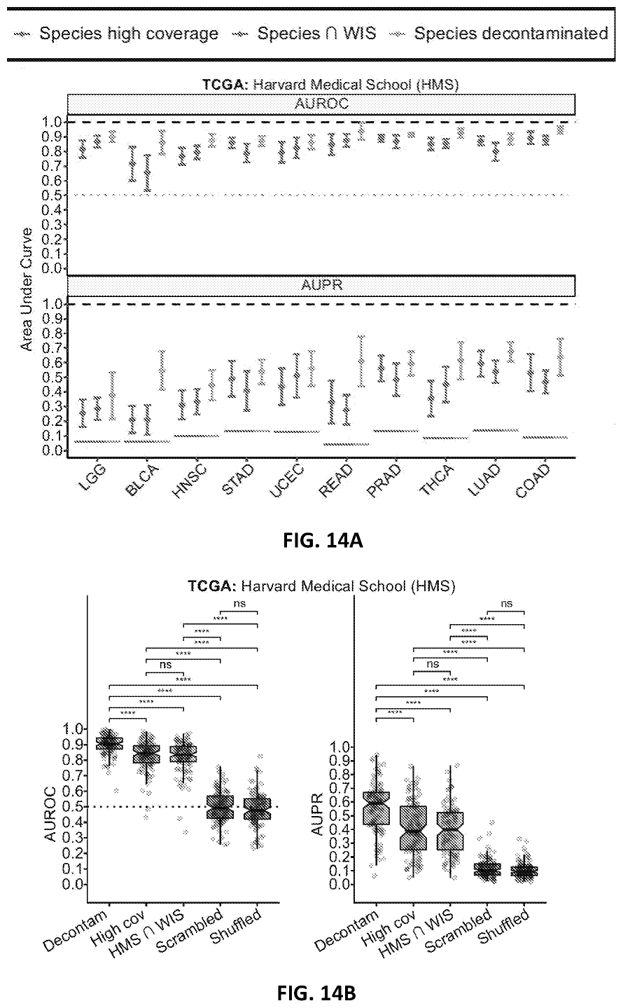

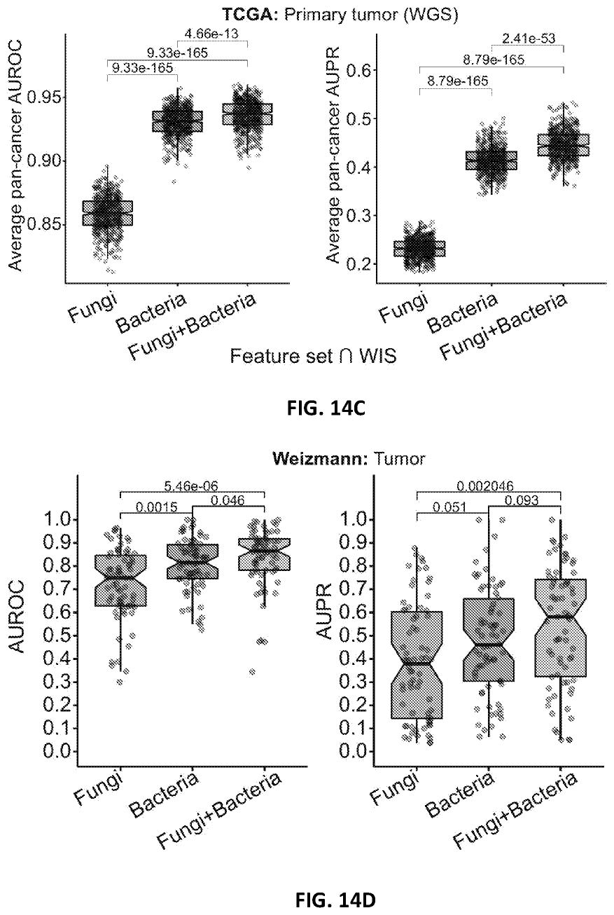

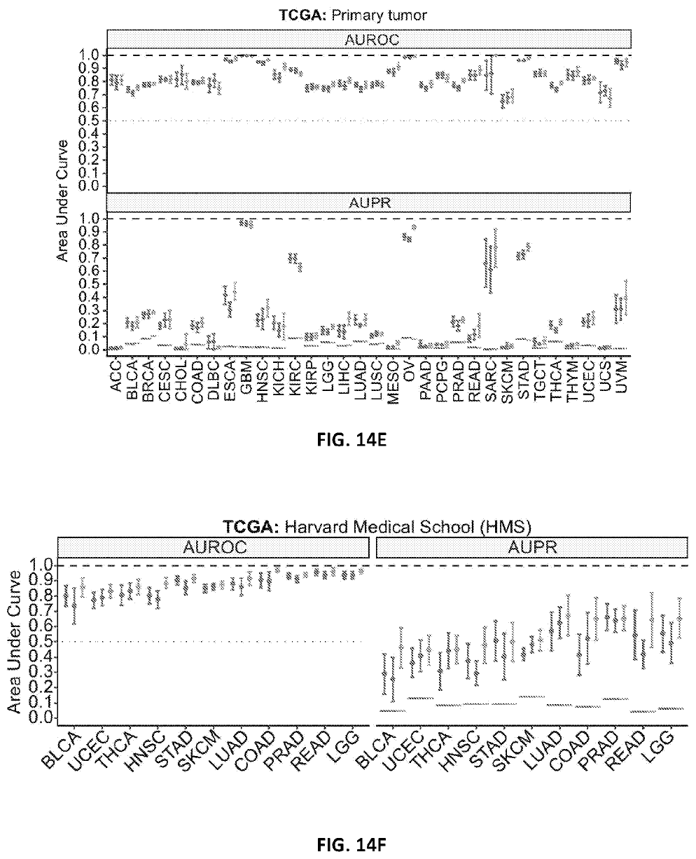

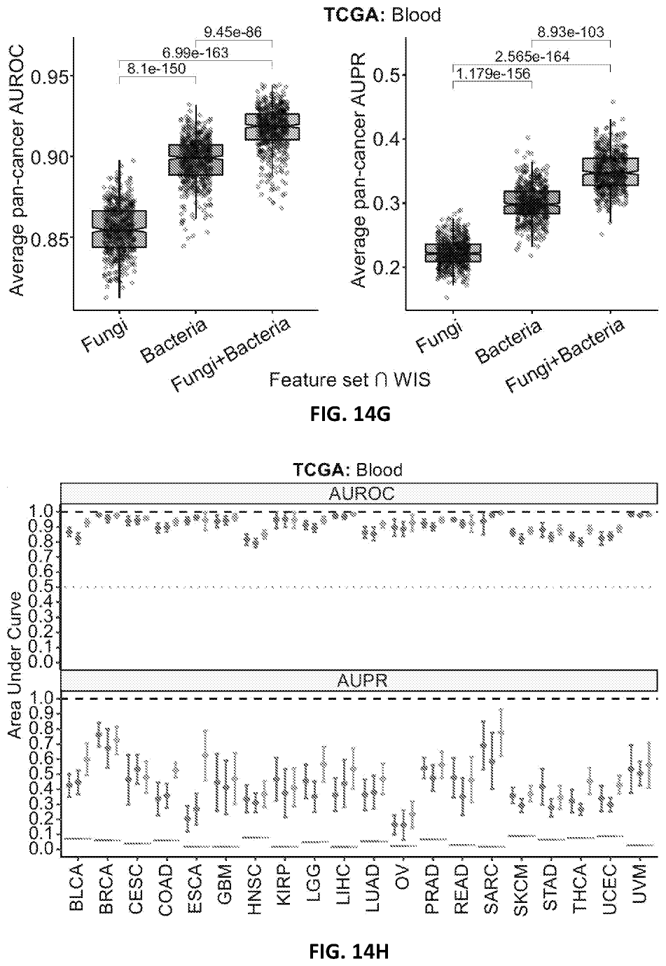

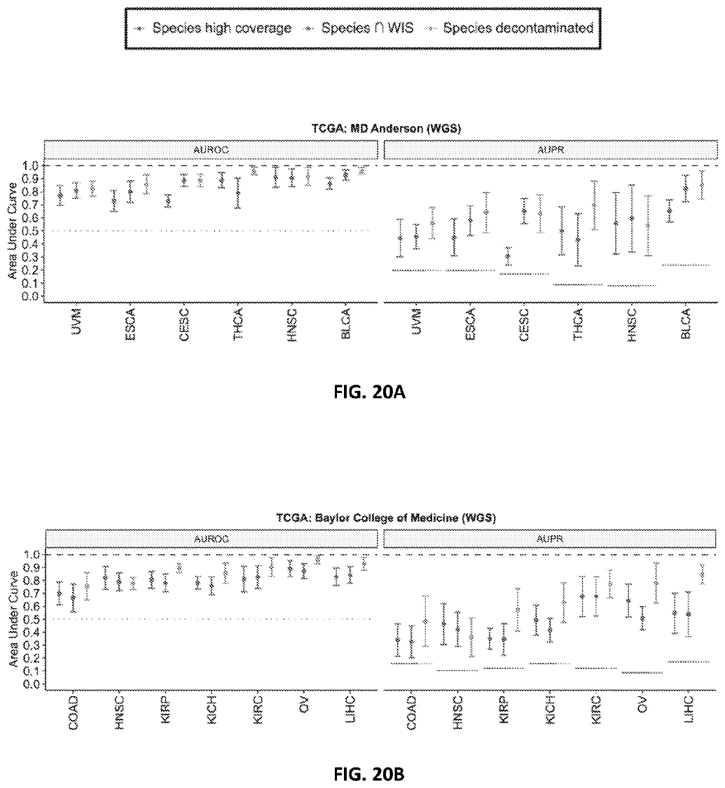

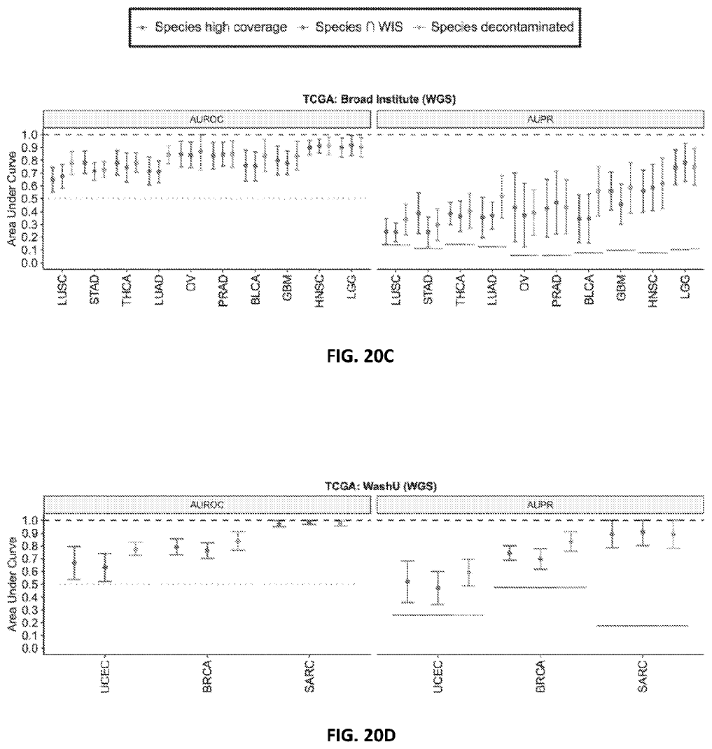

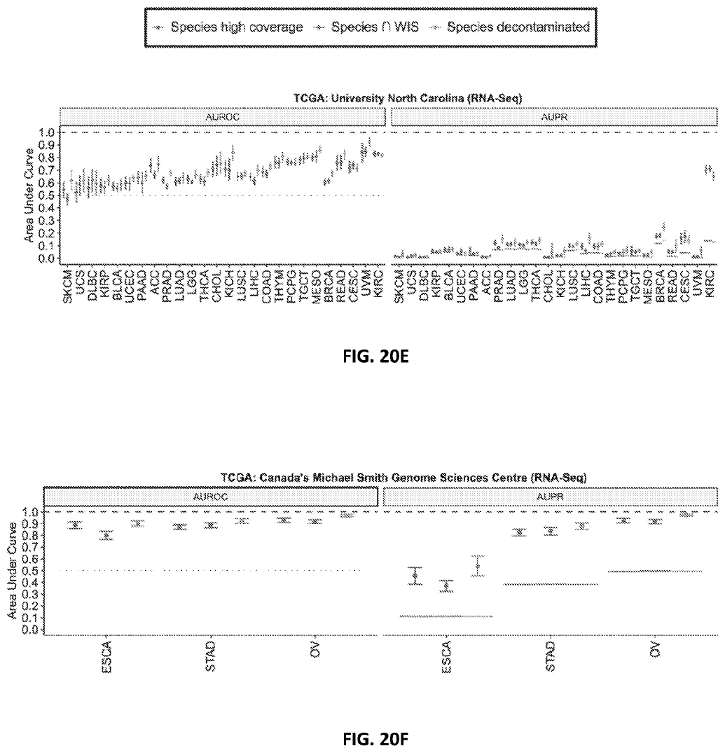

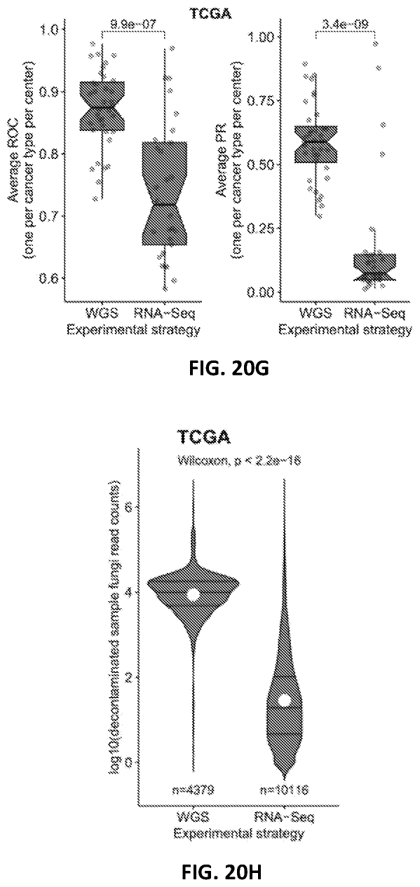

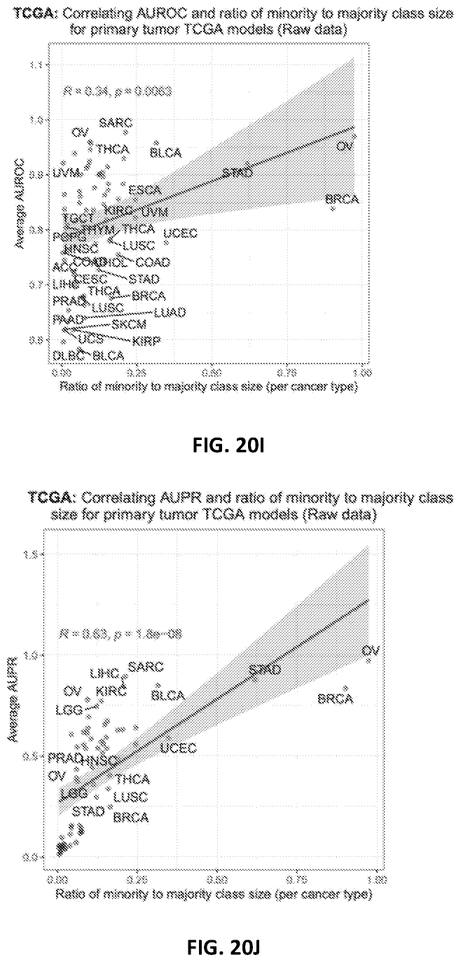

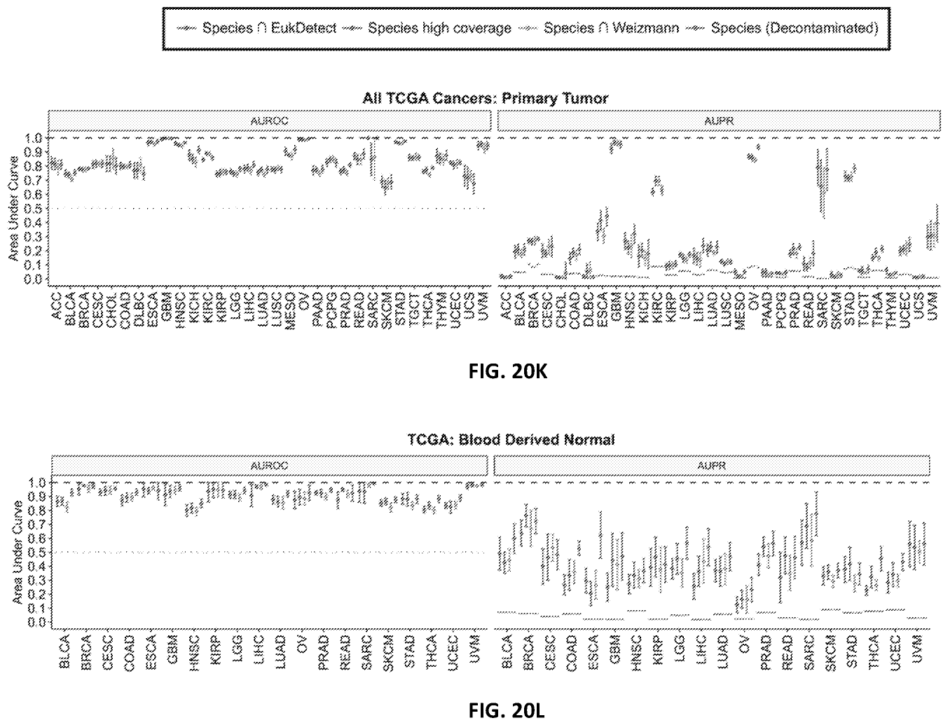

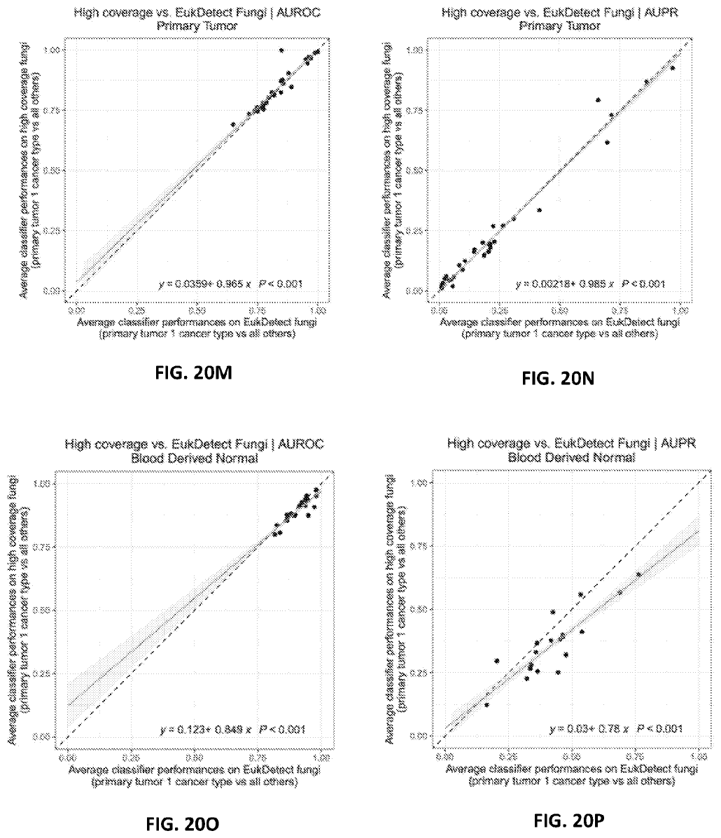

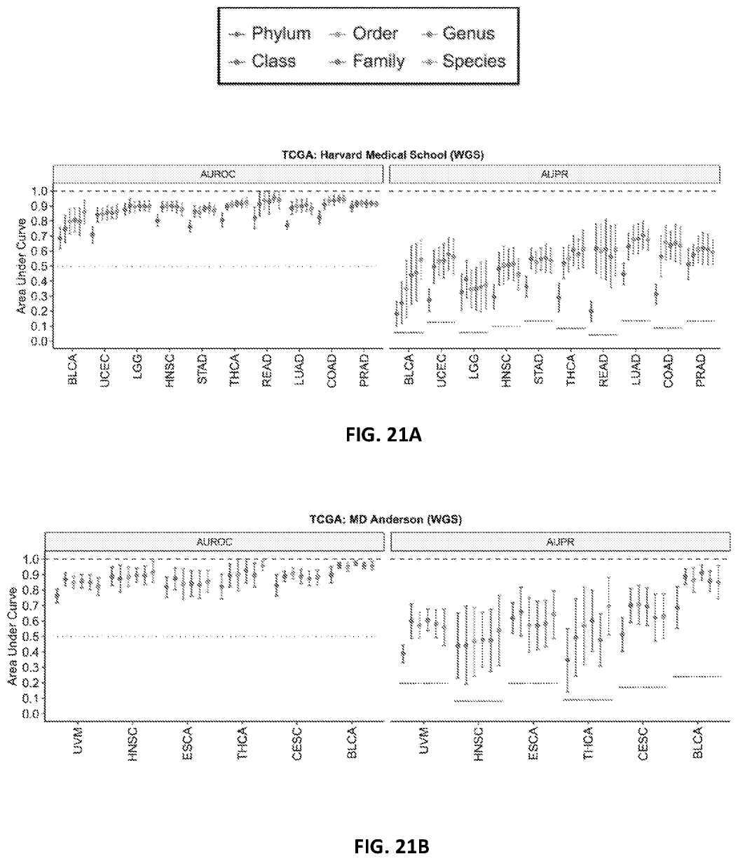

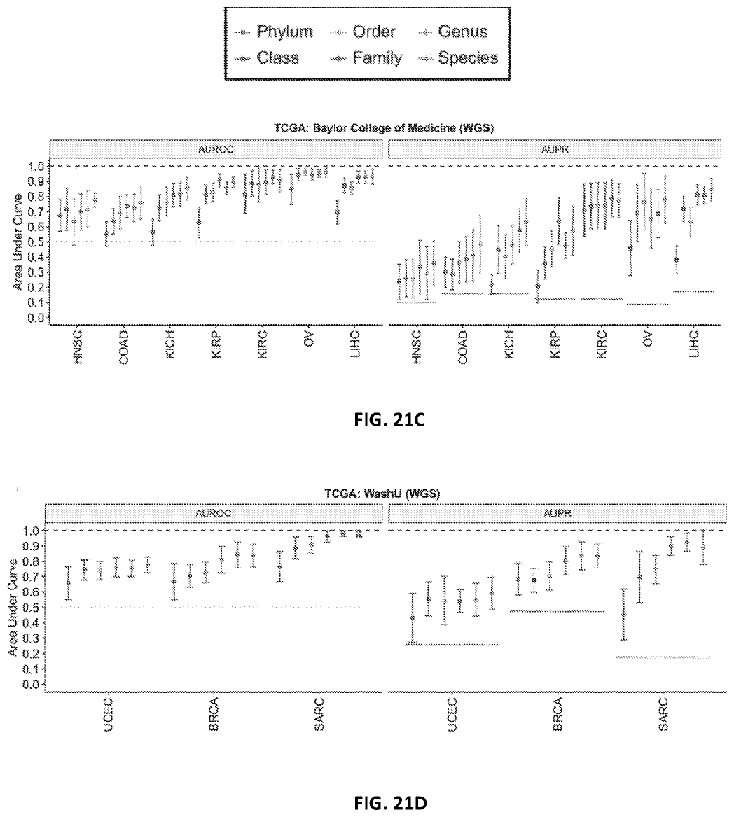

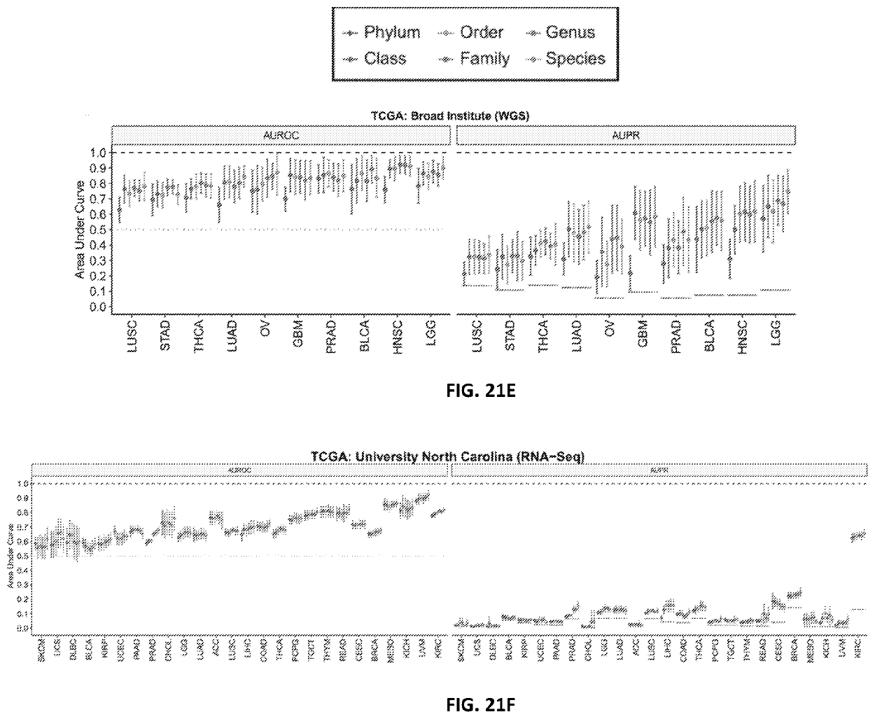

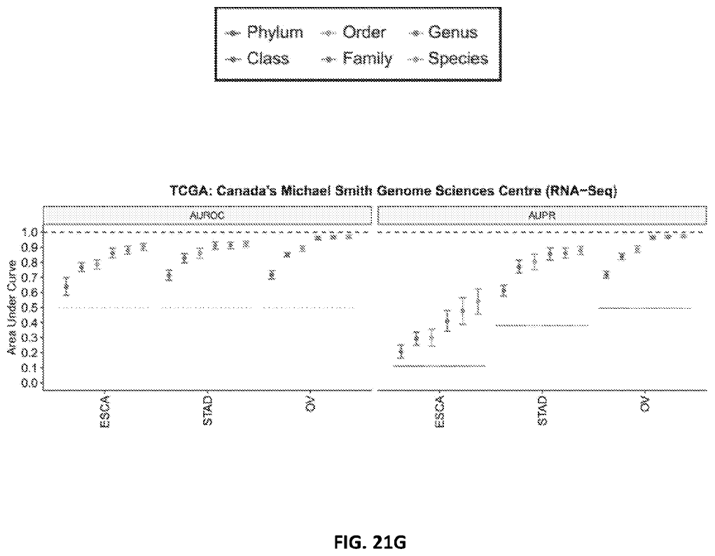

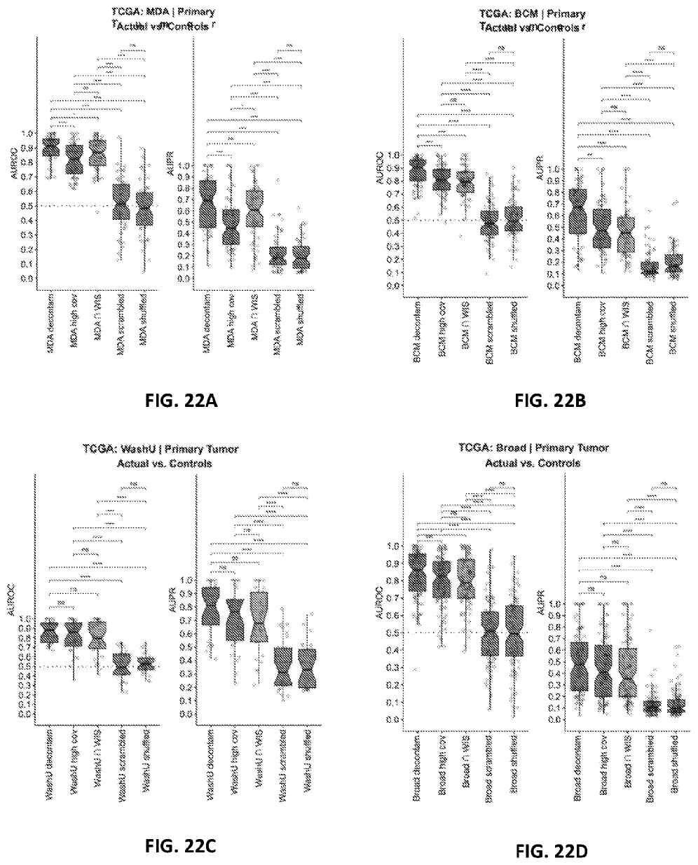

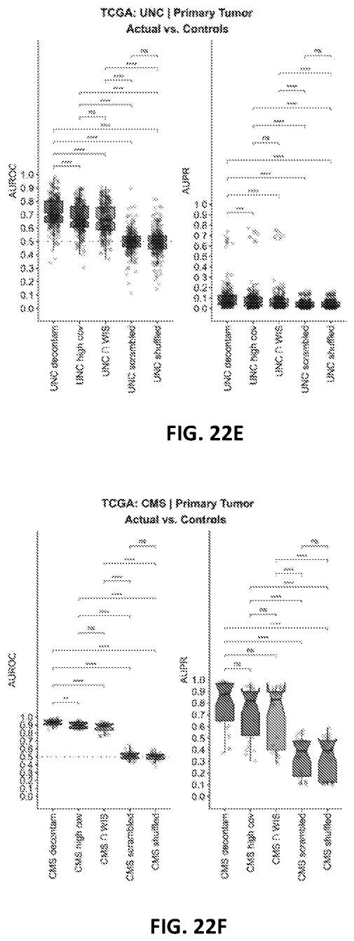

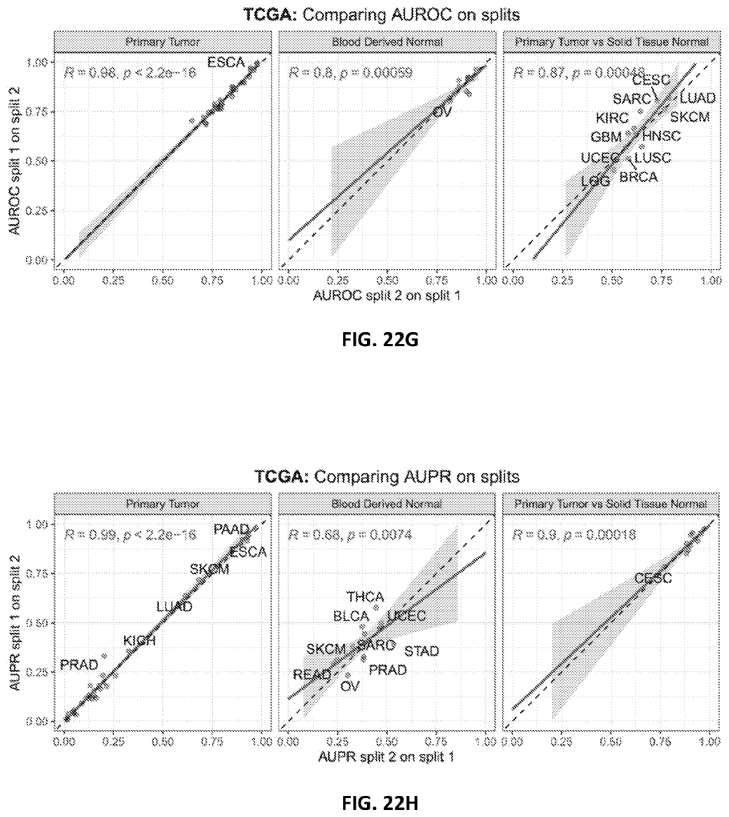

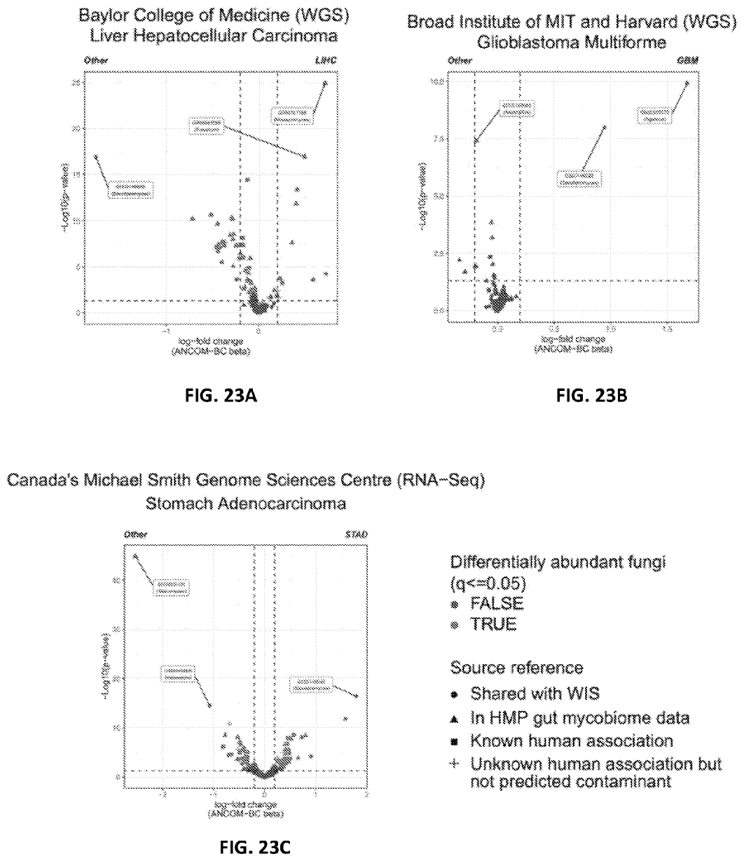

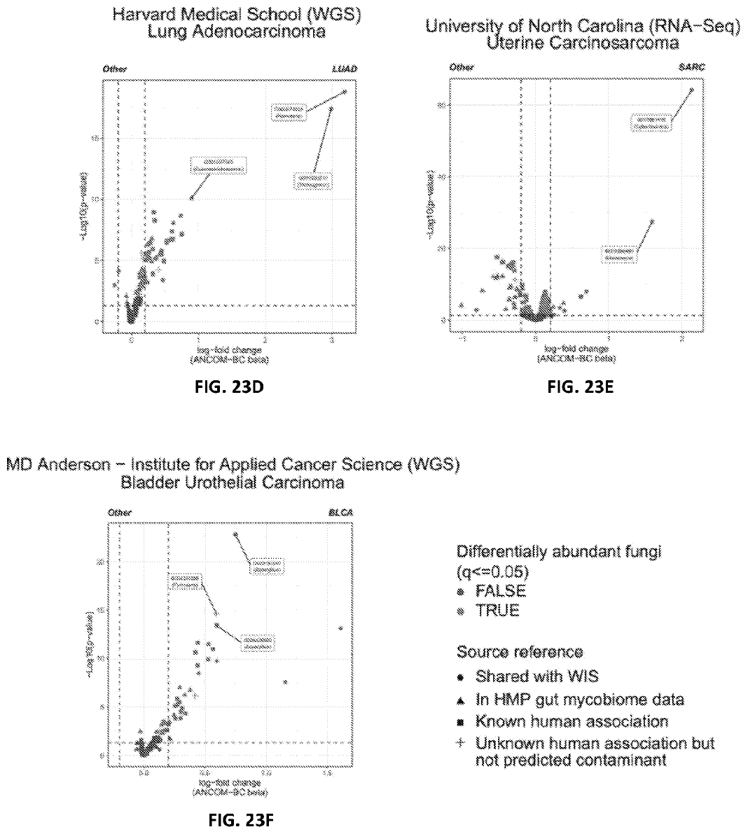

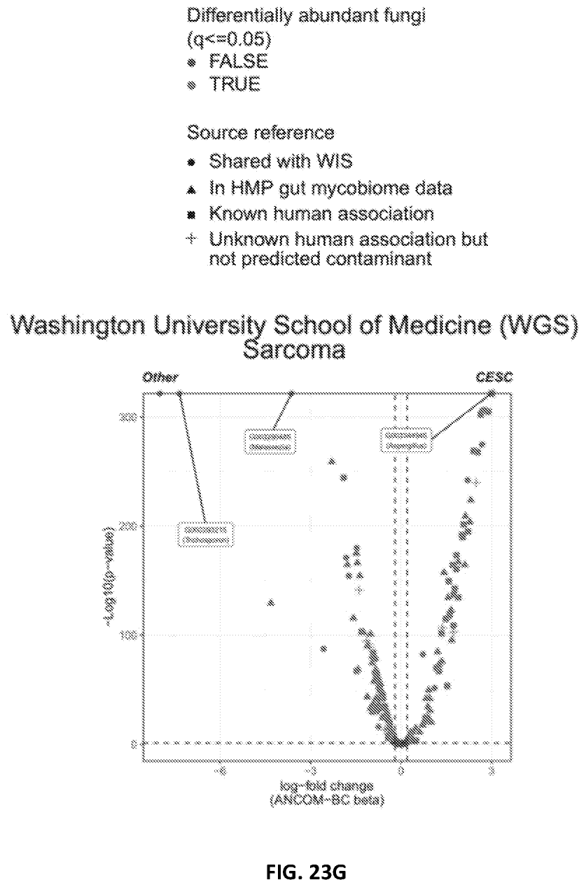

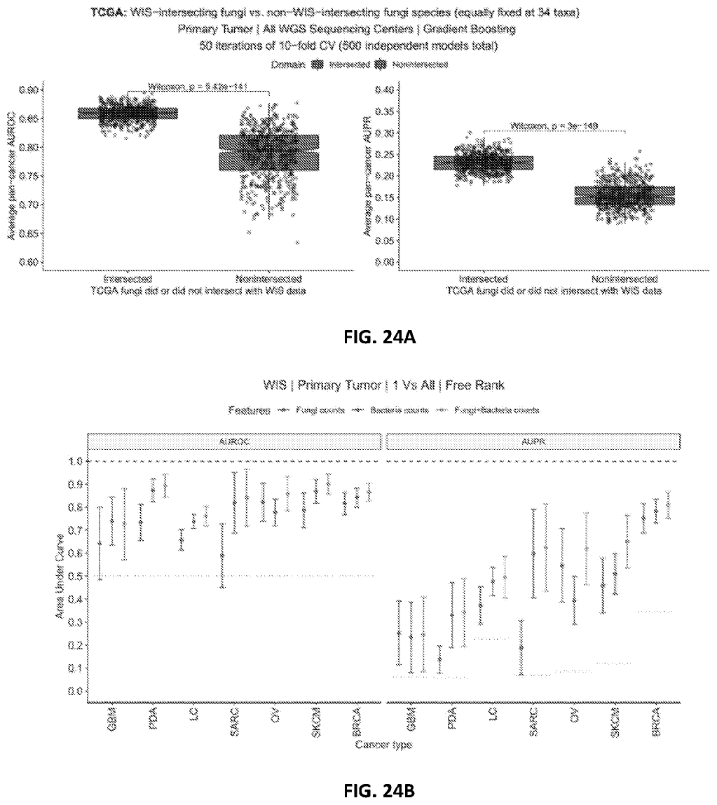

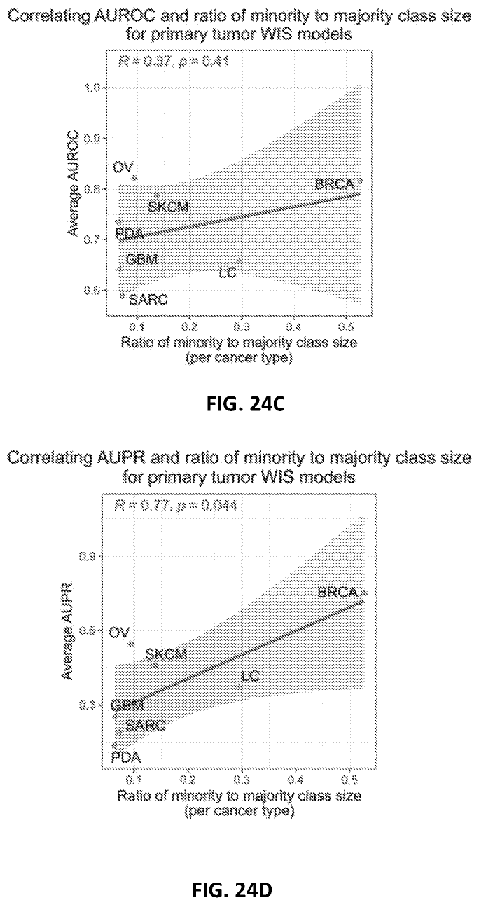

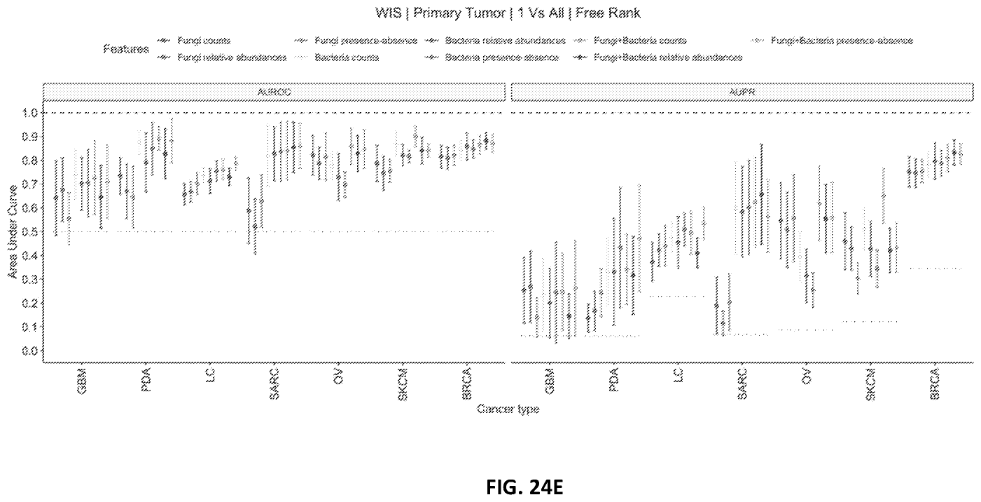

Machine learning (ML) on mycobiomes was then tested to determine if ML models trained with mycobiomes may discriminate between and within cancer types. First, ML models were evaluated on raw, decontaminated TCGA fungal count data (n=14,495 non-zero decontaminated samples) with extensive positive and negative control analyses, revealing pan-cancer discrimination, and found synergistic performance when adding bacterial information in TCGA and WIS tumors (FIGS. 14A-14D; FIGS. 20A-20P; FIGS. 21A-21G; FIGS. 22A-22H; FIGS. 23A-23G; and FIGS. 24A-24E). Towards building a pan-cancer classifier, all decontaminated TCGA mycobiome data was combined using supervised batch correction, as previously done with TCGA bacteriomes and viromes (FIG. 9A) (PMID: 32214244). Evaluating one-cancer-type-versus-all-others models on batch-corrected mycobiome species revealed strong discrimination across 32 cancer types (FIG. 14E; AUROC 95% CI: [83.27, 85.39]%). Negative controls showed null performances (FIG. 9B). Models built on two independent raw or batch-corrected TCGA halves were then cross-tested, finding significantly correlated performance among primary tumor comparisons (FIGS. 22G-22H, FIGS. 9C-9D). Subsetting the batch-corrected data to fungi identified by EukDetect (Lind and Pollard, 297 2021), a eukaryotic-specific, marker-based taxonomy assignment algorithm, gave strong performance similar to high-coverage fungi (FIGS. 20K-20P). Notably, the 31 high coverage fungi were significantly more likely to be detected by EukDetect (Fisher exact test: p=5.67×10⁻¹¹, odds ratio=28.0), suggesting that marker-based methods may be limited in low-biomass settings.

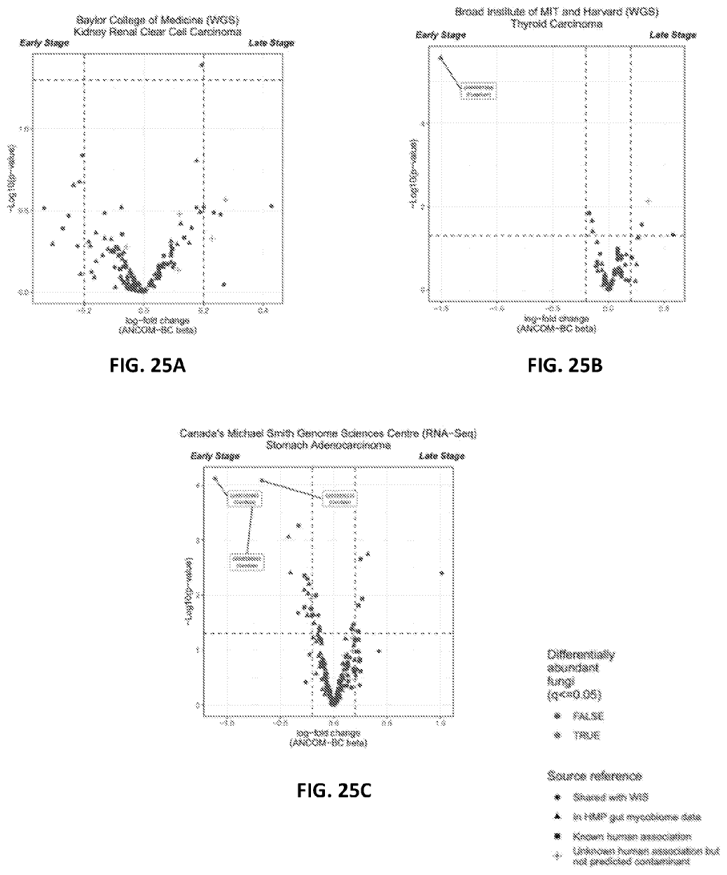

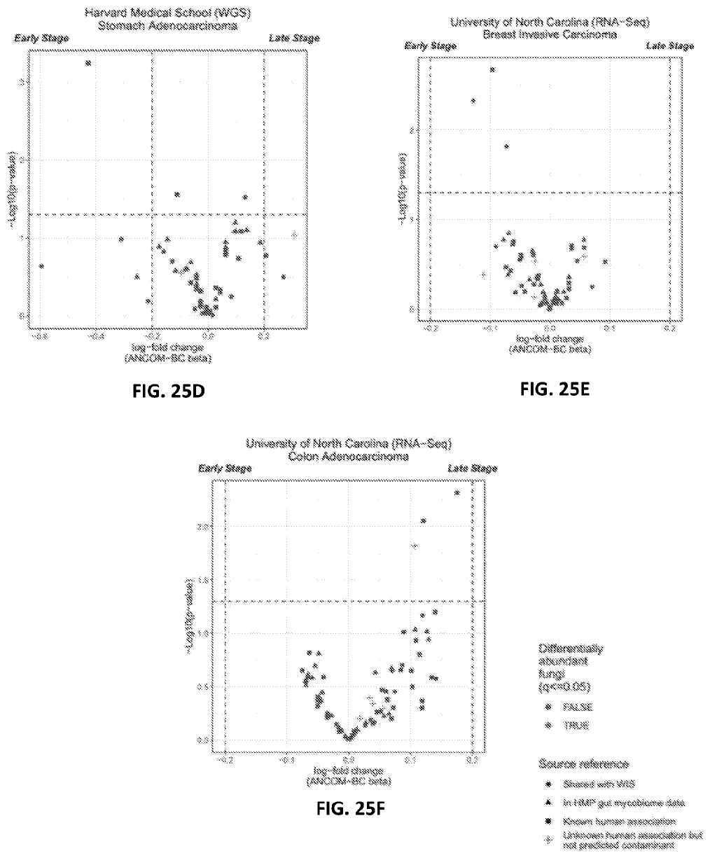

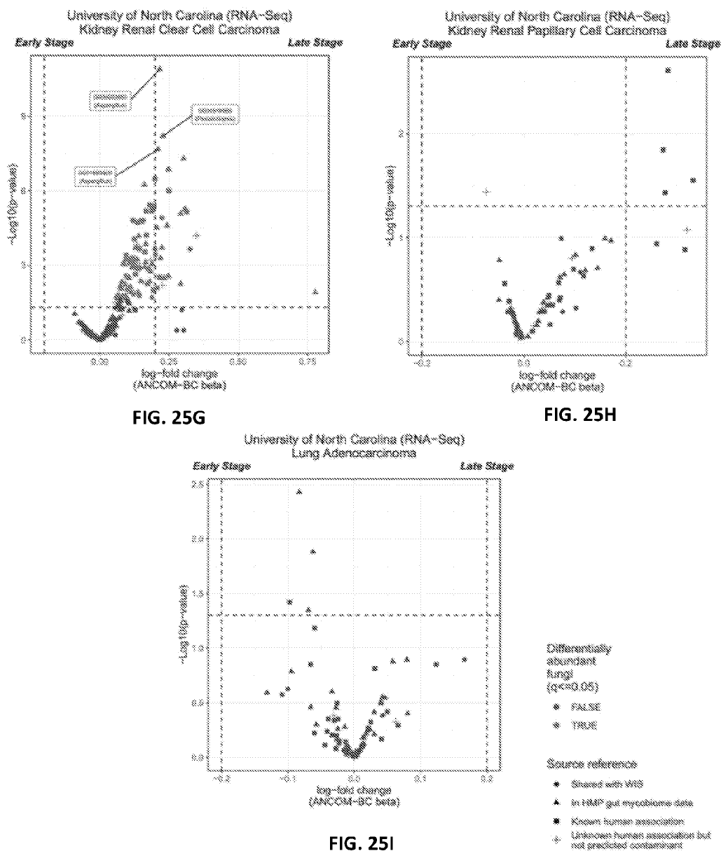

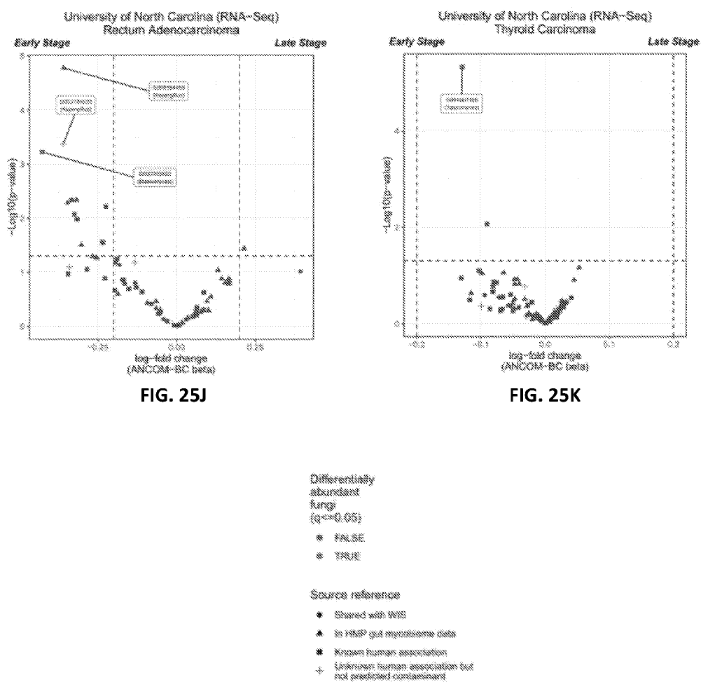

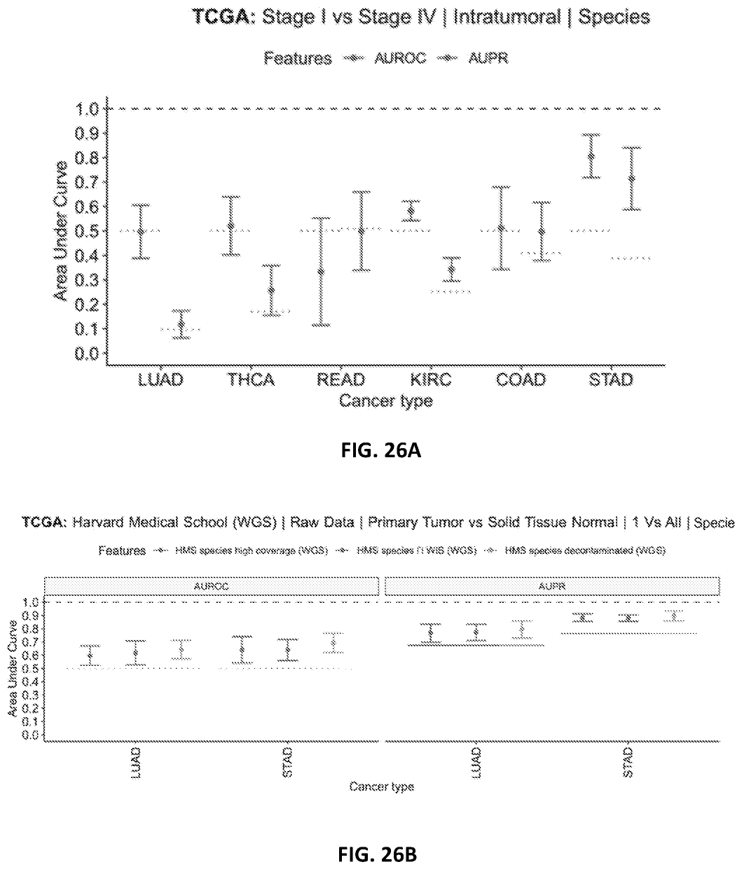

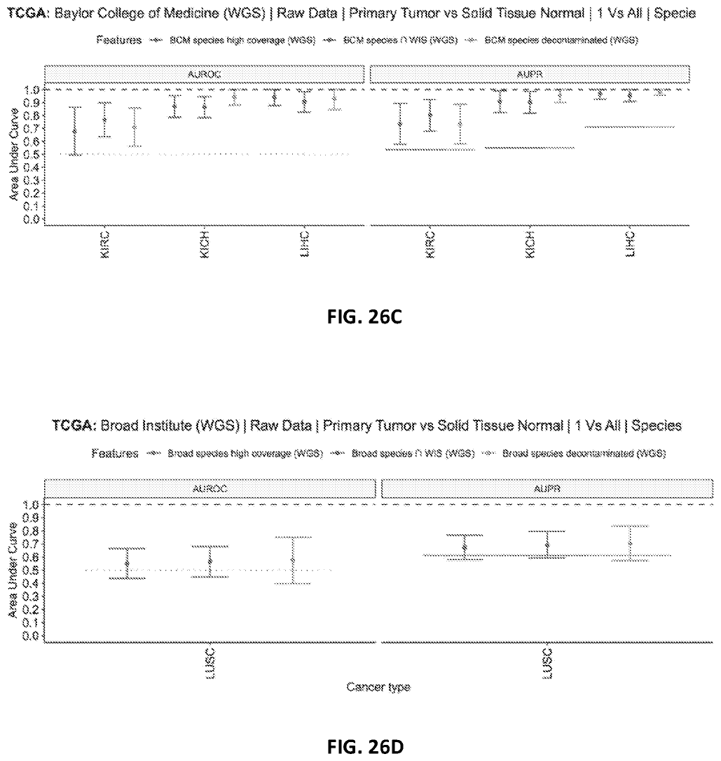

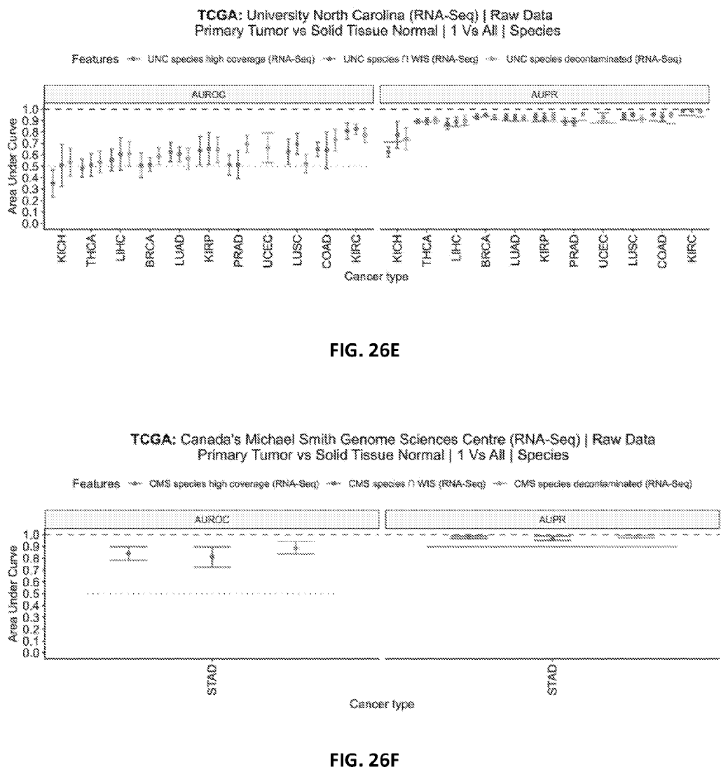

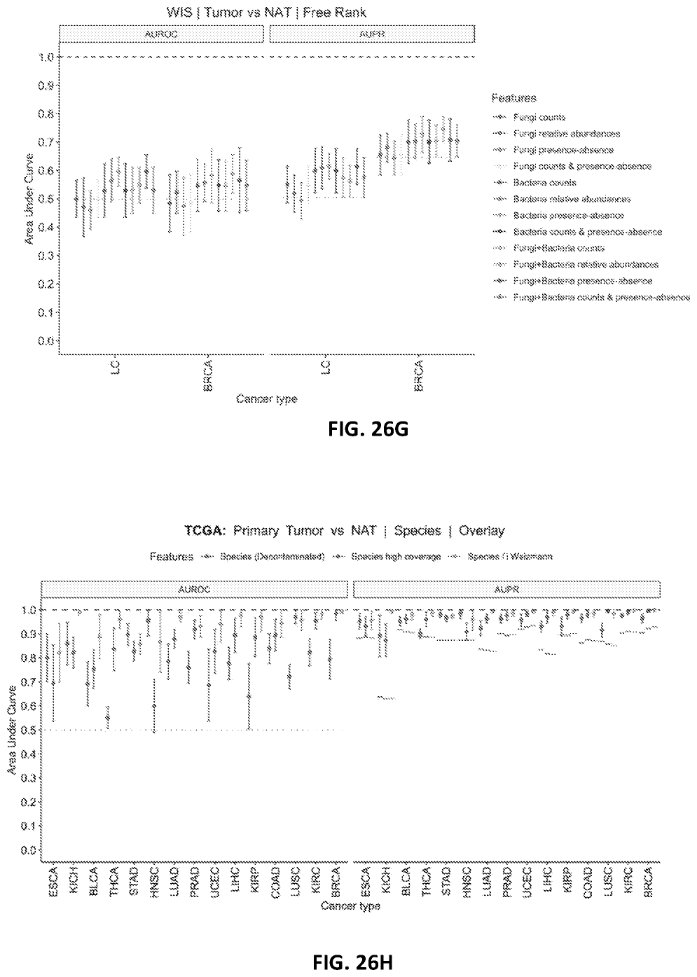

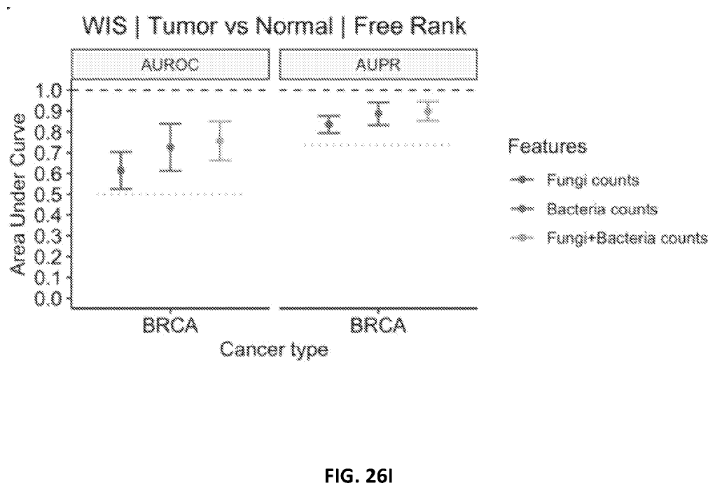

Next differential abundance (DA) testing and ML between stage I and stage IV tumor mycobiomes was conducted. DA testing revealed stage-specific fungi for stomach, rectal, and renal cancers among RNA-Seq samples (FIGS. 25A-25K), and ML supported stomach and renal cancer stage differentiation (FIG. 26A), agreeing with previous results on stage-specific bacteriomes excluding colon cancer.

Tumor and NAT mycobiome samples are similar in composition, so discriminating them may be hard. Tumor vs. NAT ML performed poorly on most TCGA raw data subsets and WIS data (FIGS. 26B-26G). Stomach and kidney cancers may comprise exceptions (FIGS. 26B, 26C, 26E, and 26F) but were absent in the WIS cohort. Nonetheless, the small tumor-NAT effect size seemed surmountable when re-examining the full, batch corrected dataset (FIG. 26H). Analogously, comparing breast tumors to true normal tissue in the WIS cohort revealed differential fungal prevalence and better ML performance (FIG. 26I). These analyses suggest tissue mycobiomes may distinguish tumor and NAT in sufficiently powered studies.

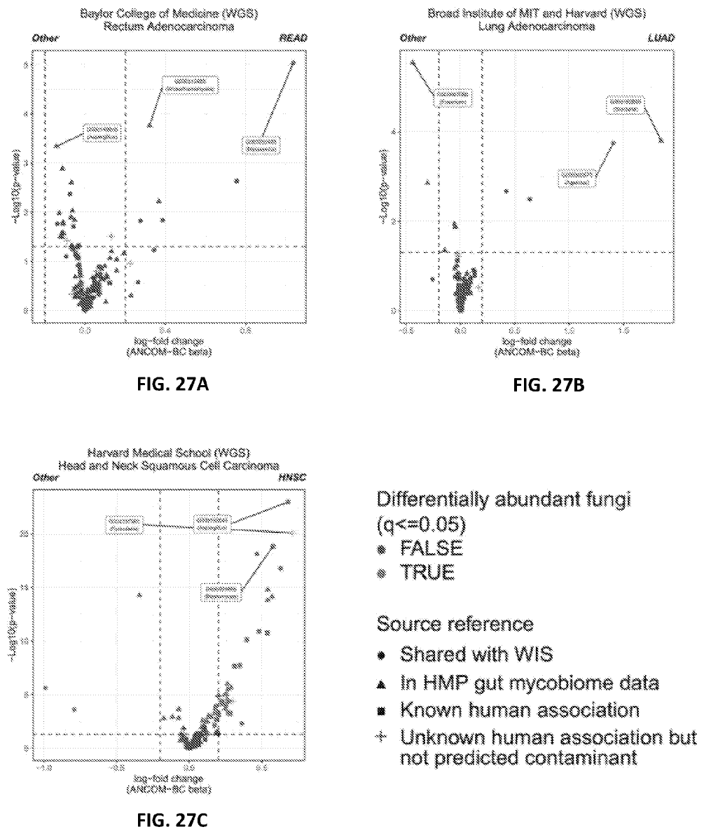

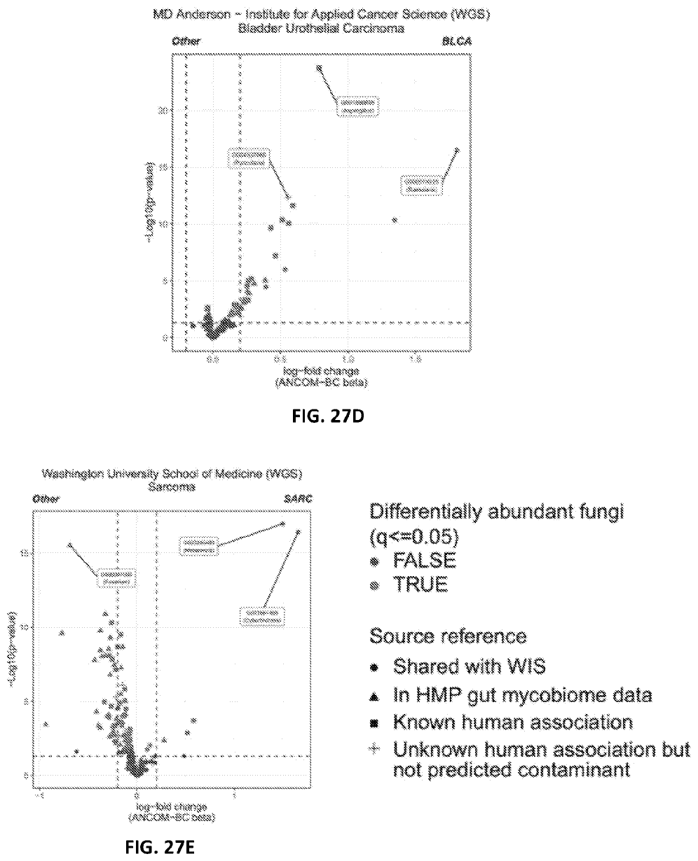

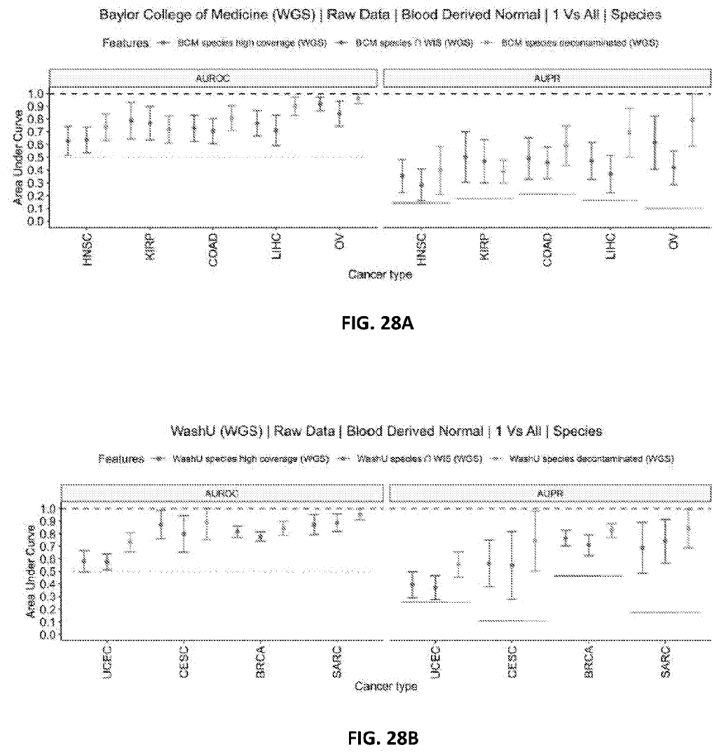

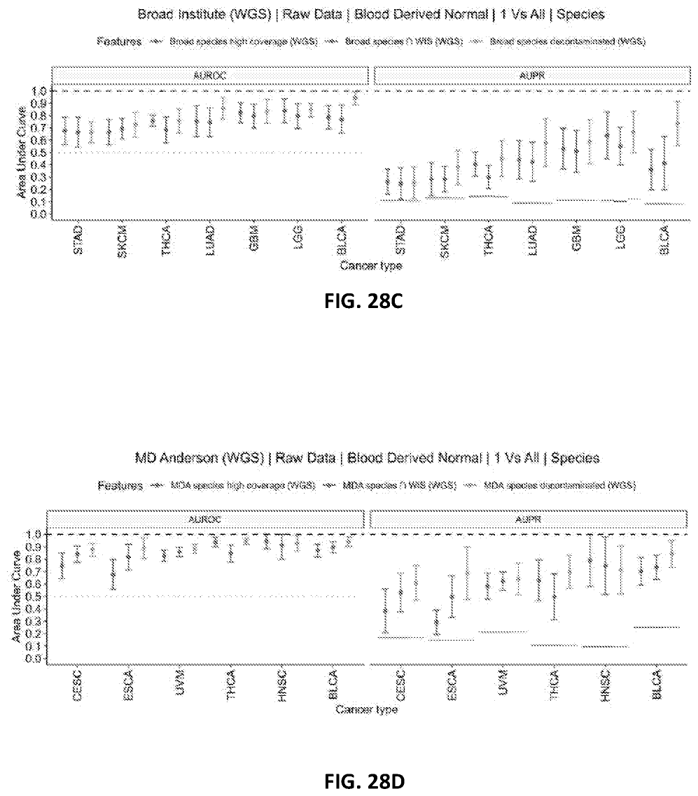

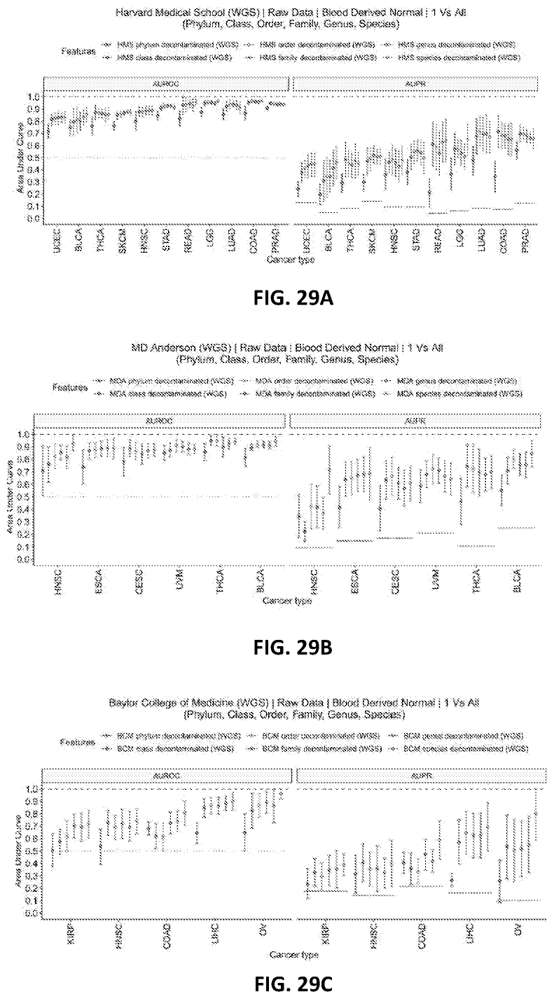

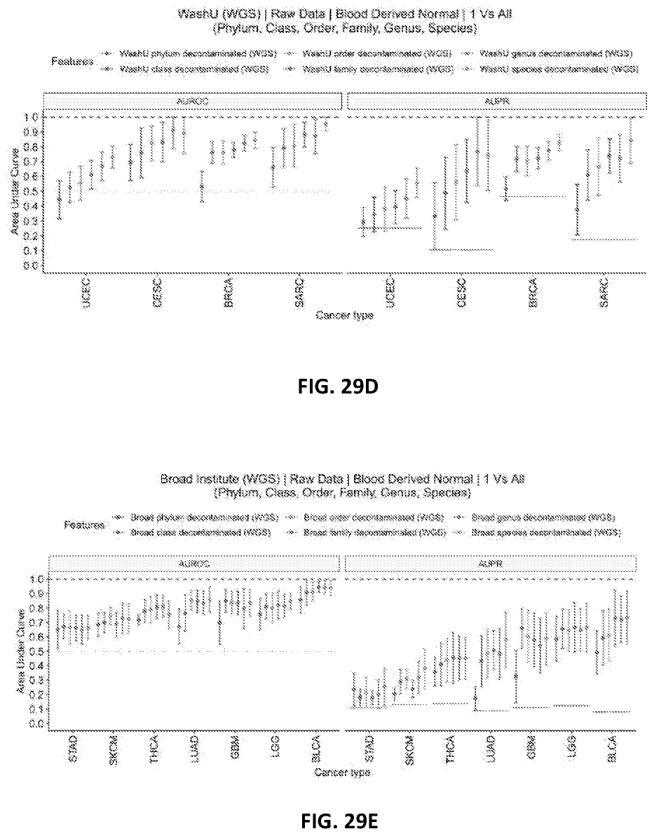

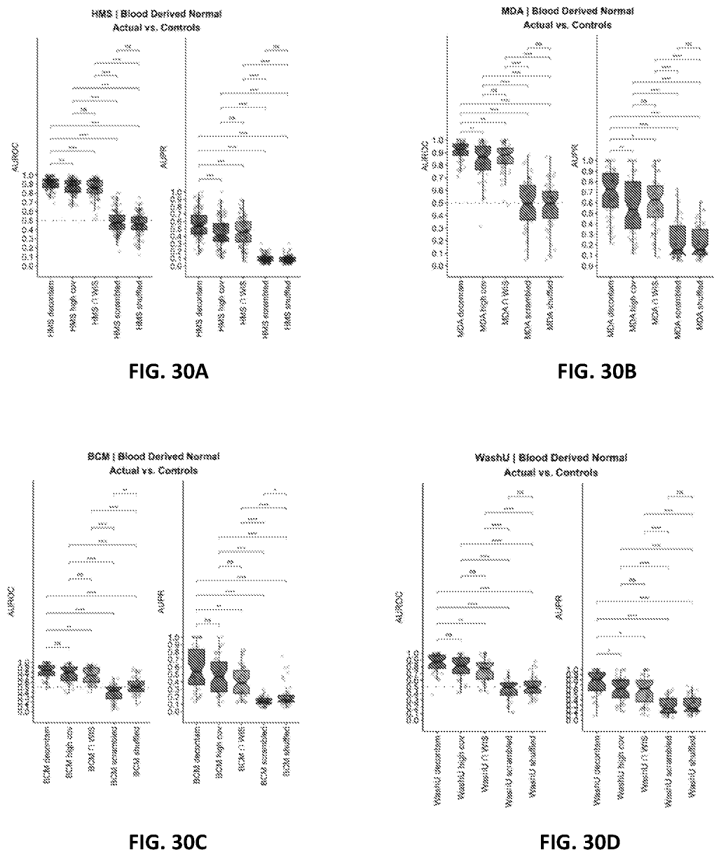

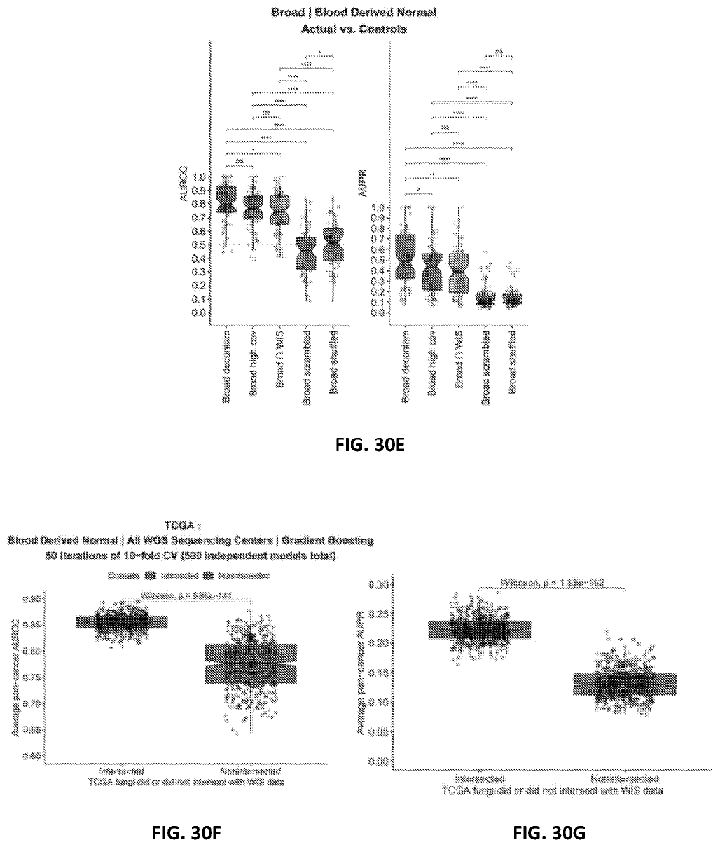

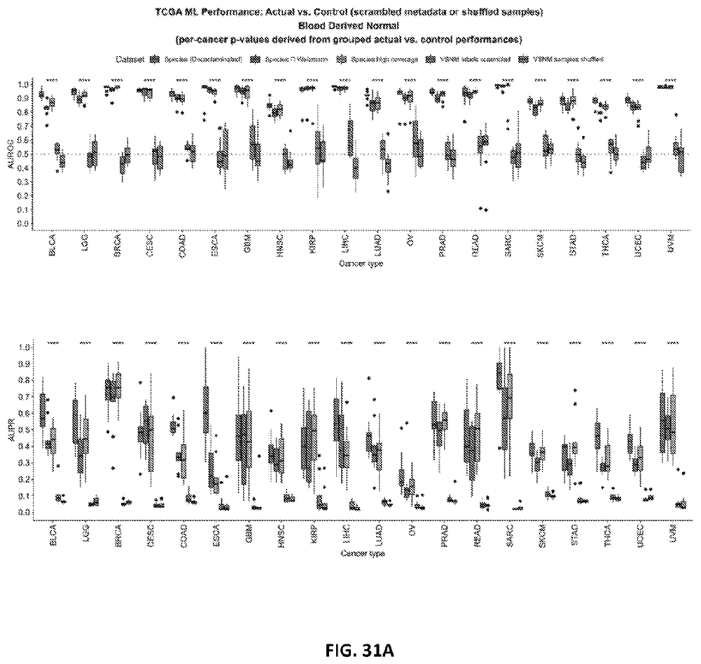

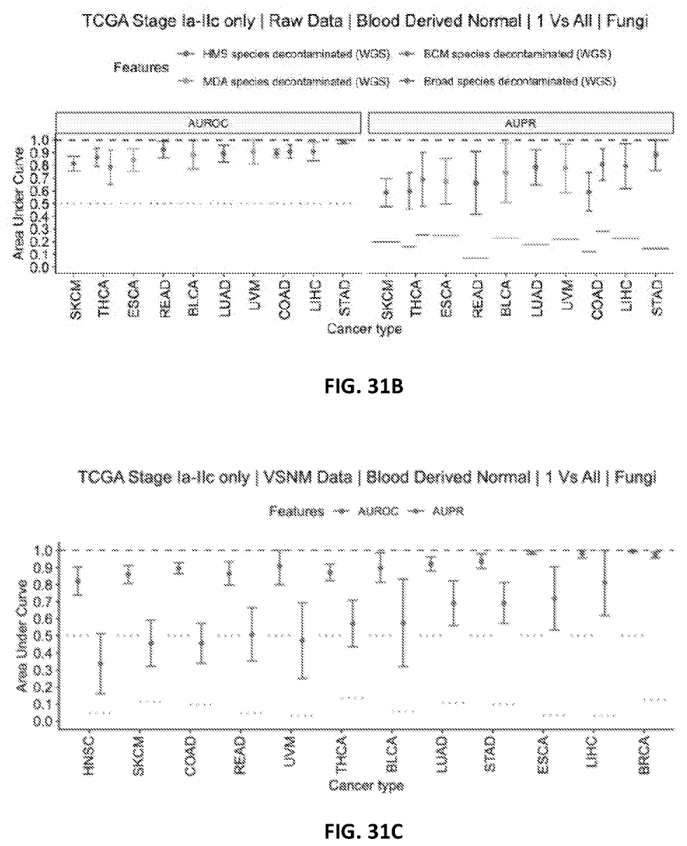

Previous bacteriome-centric analyses revealed cancer type-specific, blood-derived microbial DNA, prompting an examination of fungal DNA in TCGA WGS blood samples. DA testing and ML on raw, decontaminated fungal data with extensive controls showed strong discrimination between cancer types and synergy with bacterial features (FIG. 14F-14G; FIGS. 27A-27E; FIGS. 28A-28D; FIGS. 29A-29E; and FIGS. 30A-30G). ML on batch-corrected fungal species also showed pan-cancer discrimination (AUROC 95% CI: [92.42, 94.02]%; FIG. 14H) with null performance on negative controls (FIG. 31A). Subsetting the analysis to stage Ia-IIc cancers in raw and batch corrected datasets suggested stage-invariant performance (FIGS. 31B-31C).

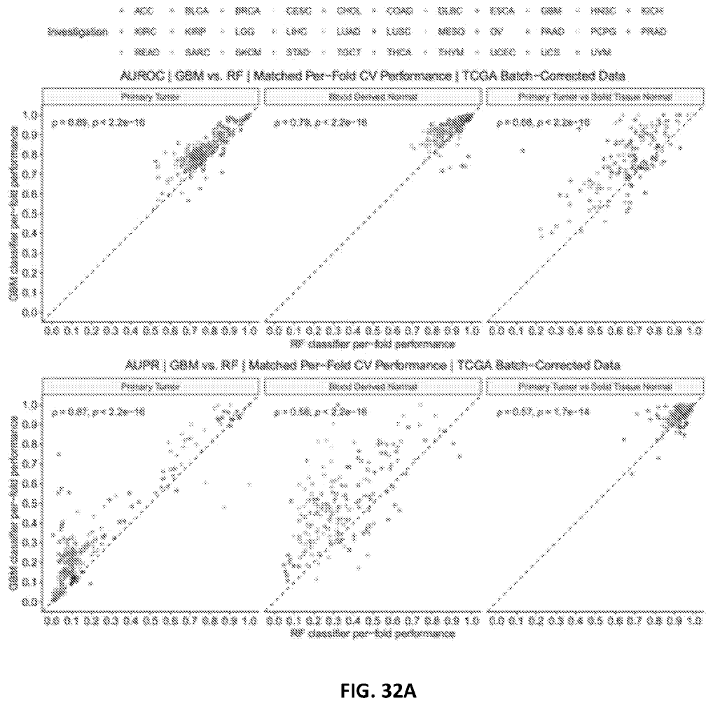

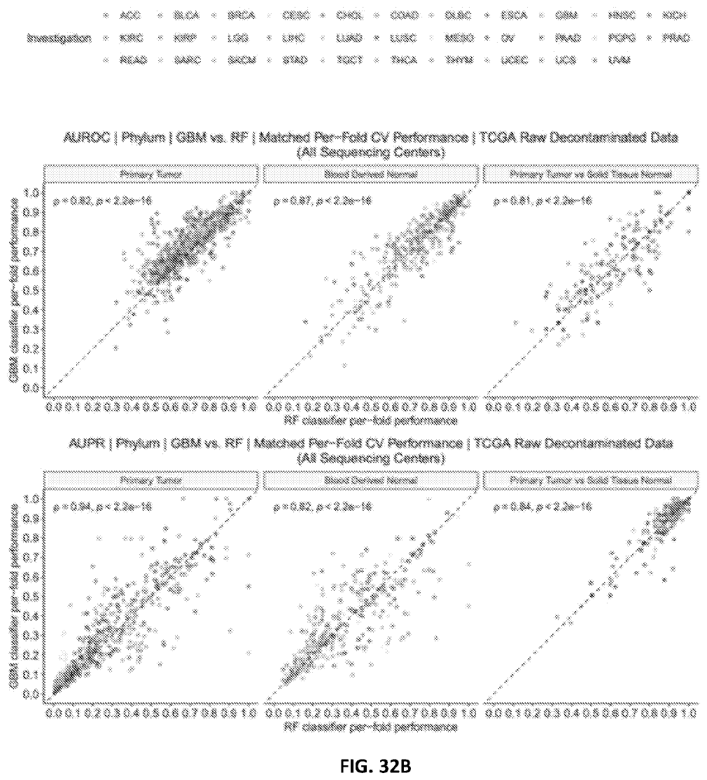

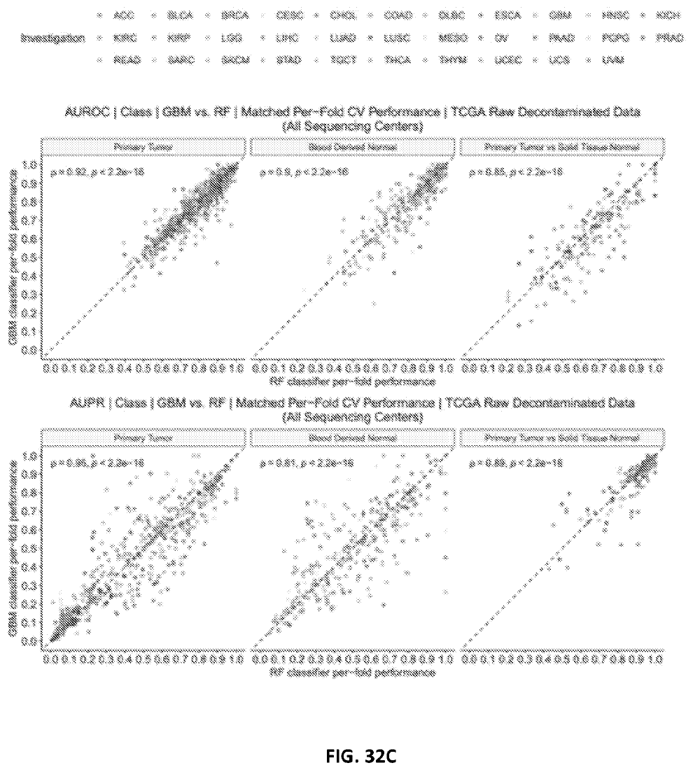

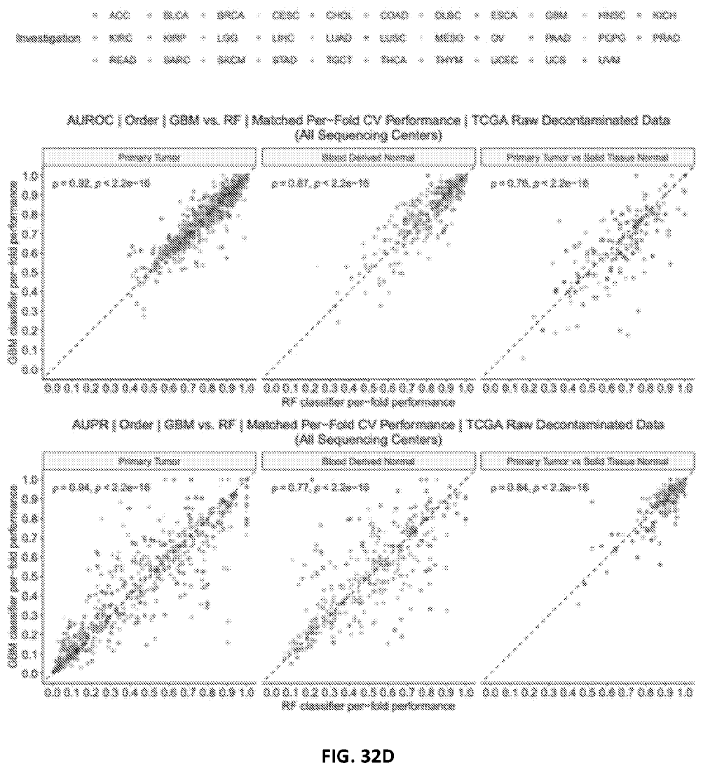

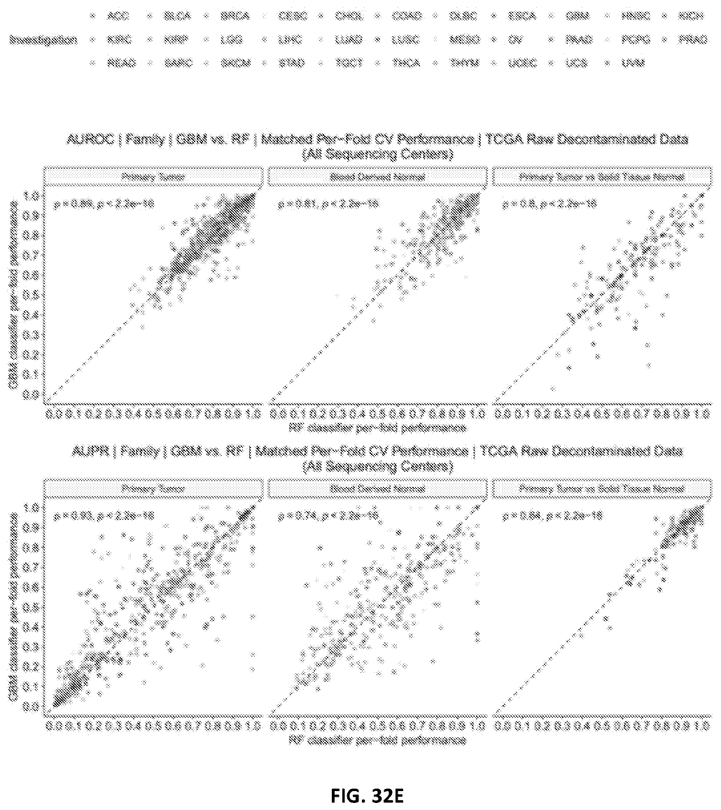

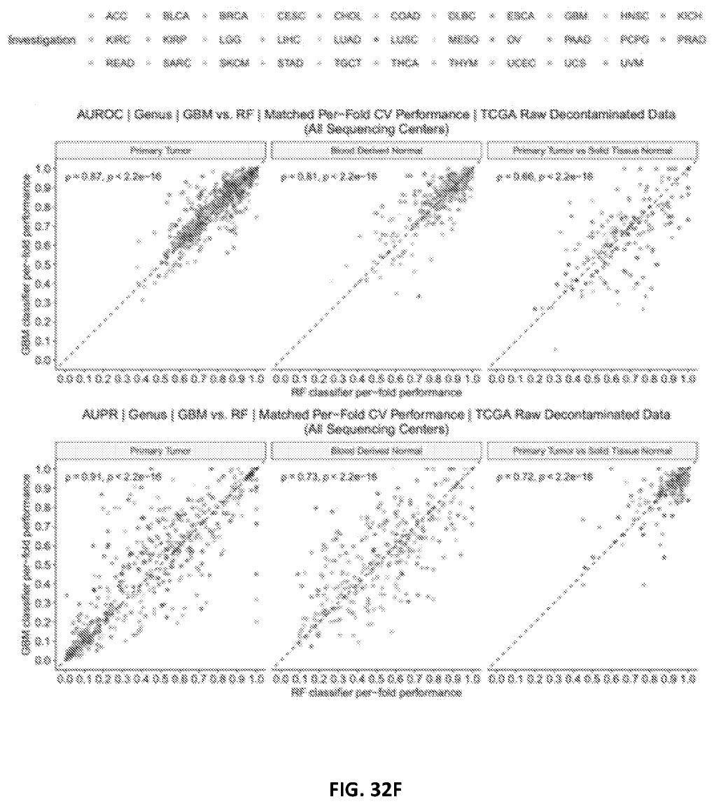

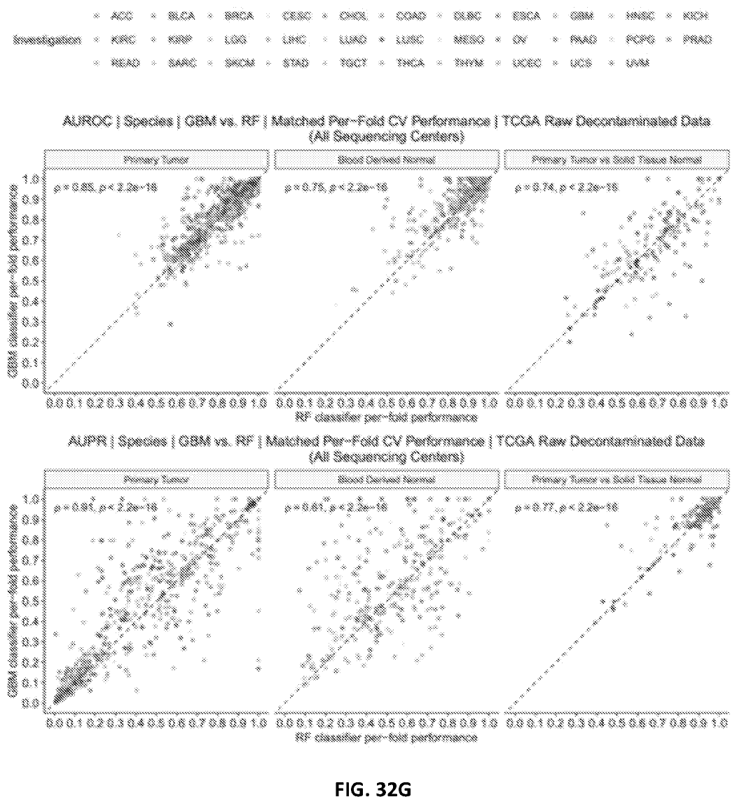

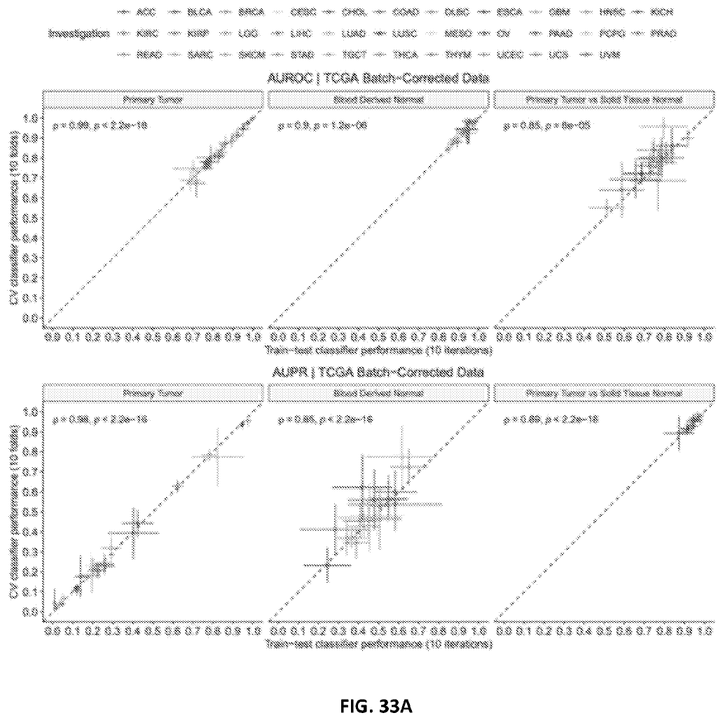

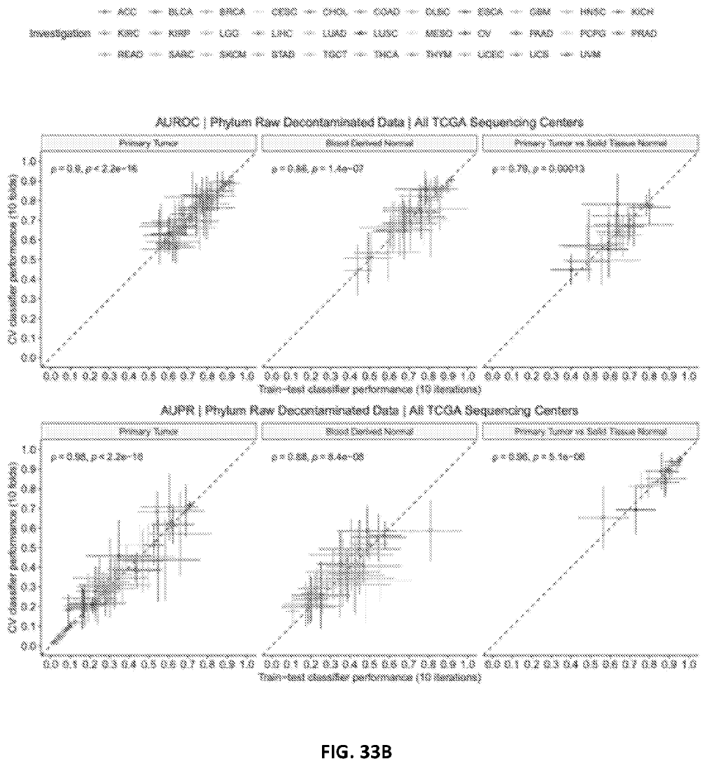

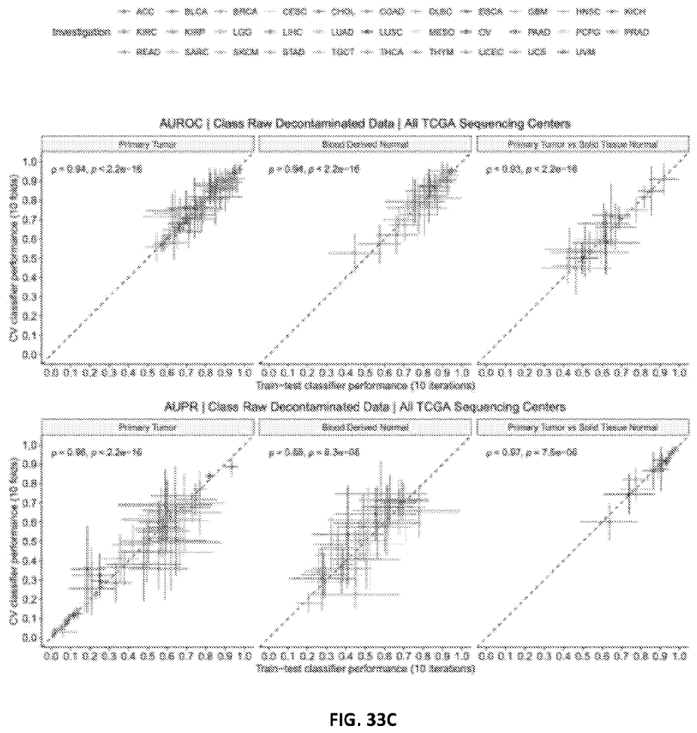

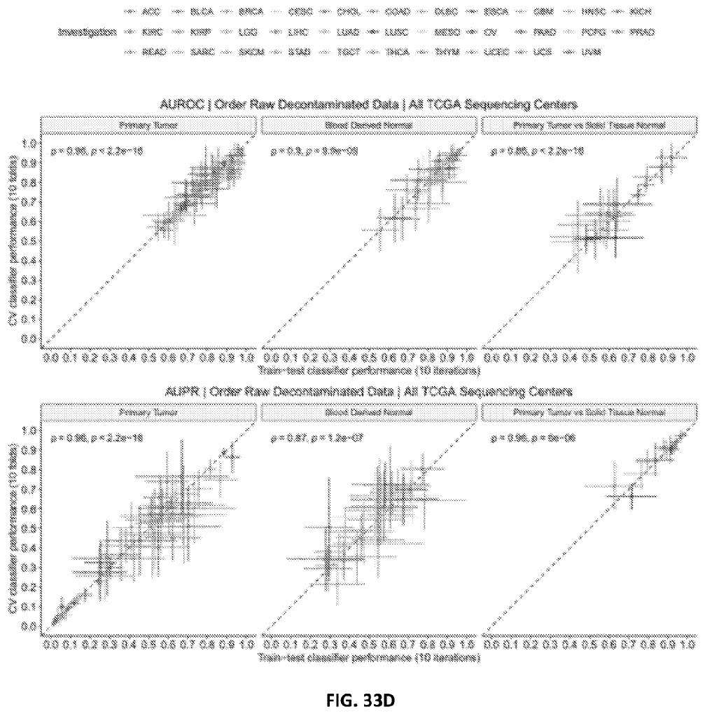

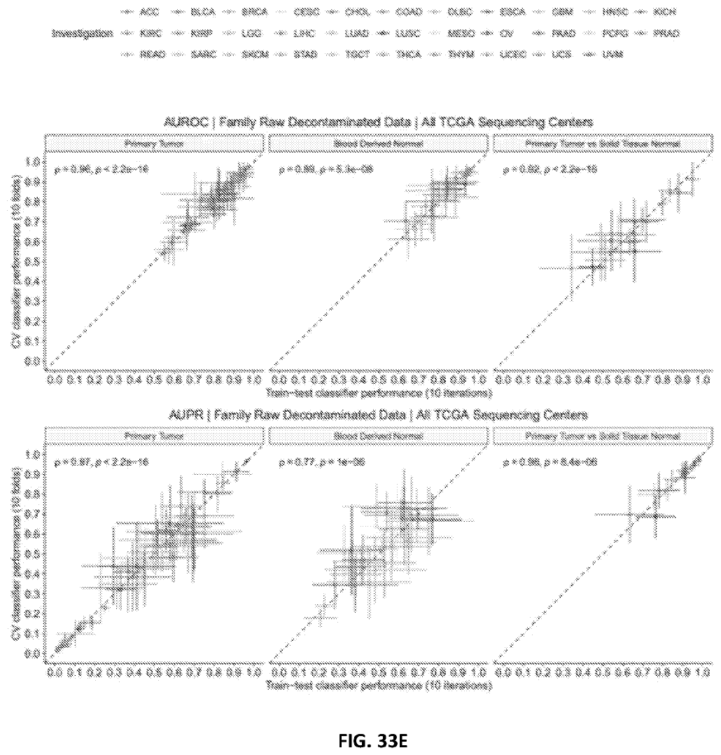

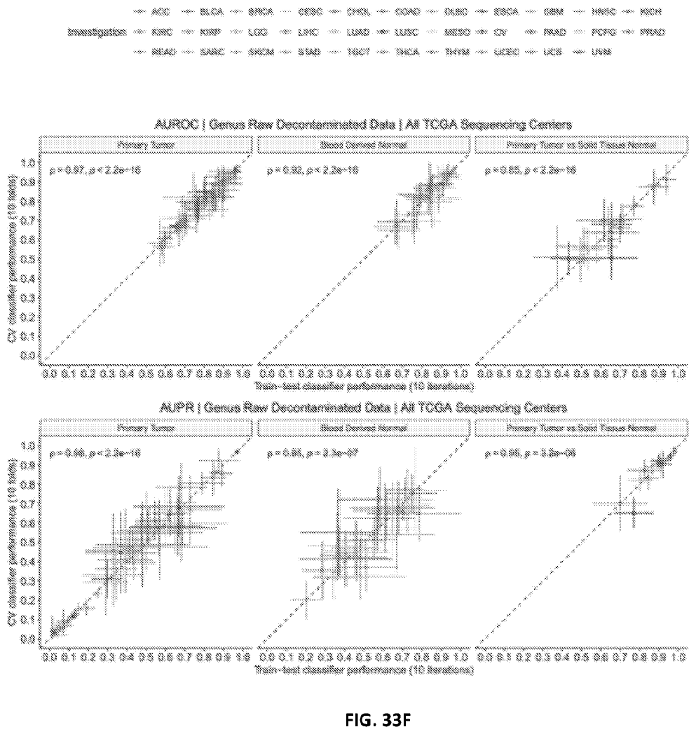

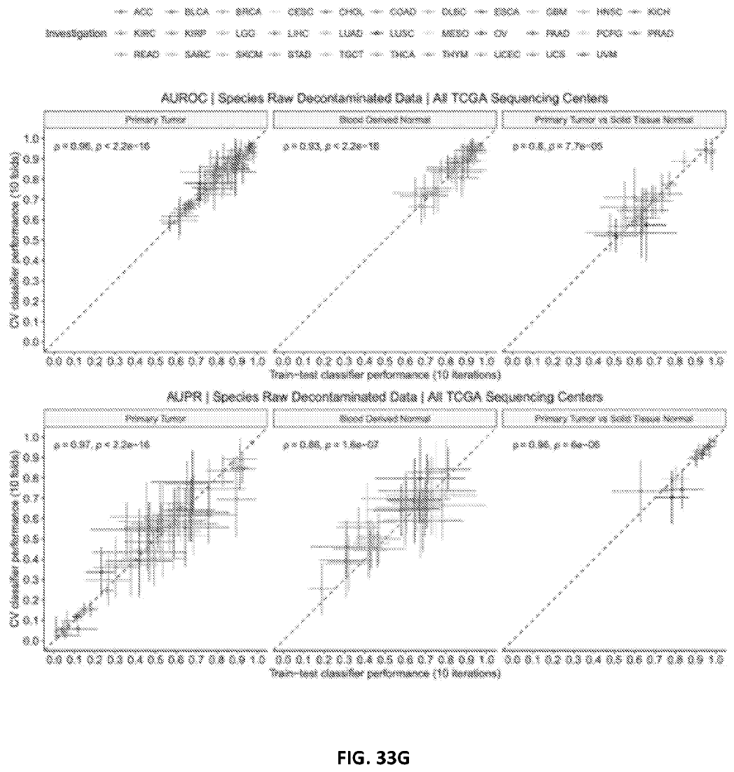

All raw and batch-corrected tumor, blood, and NAT analyses was then repeated using differing ML model types and sampling strategies, finding similar results (FIGS. 32A-32G; and FIGS. 33A-33G), suggesting generalizable performance. Statistical and ML analyses support cancer-type specific tissue and blood mycobiomes, with potential clinical utility.

### Clinical Utility of Cancer Mycobiomes

Blood-derived, stage-invariant, cancer-type specific fungal compositions in TCGA suggest their utility as minimally-invasive diagnostics, analogous to bacterial counterparts. These findings were validated in two independent, published cohorts (Hopkins, UCSD) comprising in aggregate 330 healthy and 376 cancer-bearing subjects that underwent shallow whole genome plasma sequencing. The Hopkins cohort focused on treatment-naive, early-stage cancers while the UCSD cohort focused on treated, late-stage cancers, collectively addressing most clinical scenarios across 10 cancer types. Additionally, the Hopkins cohort benchmarked well established, state-of-the-art fragmentomic diagnostics, providing direct performance comparisons to microbial-centric methods.

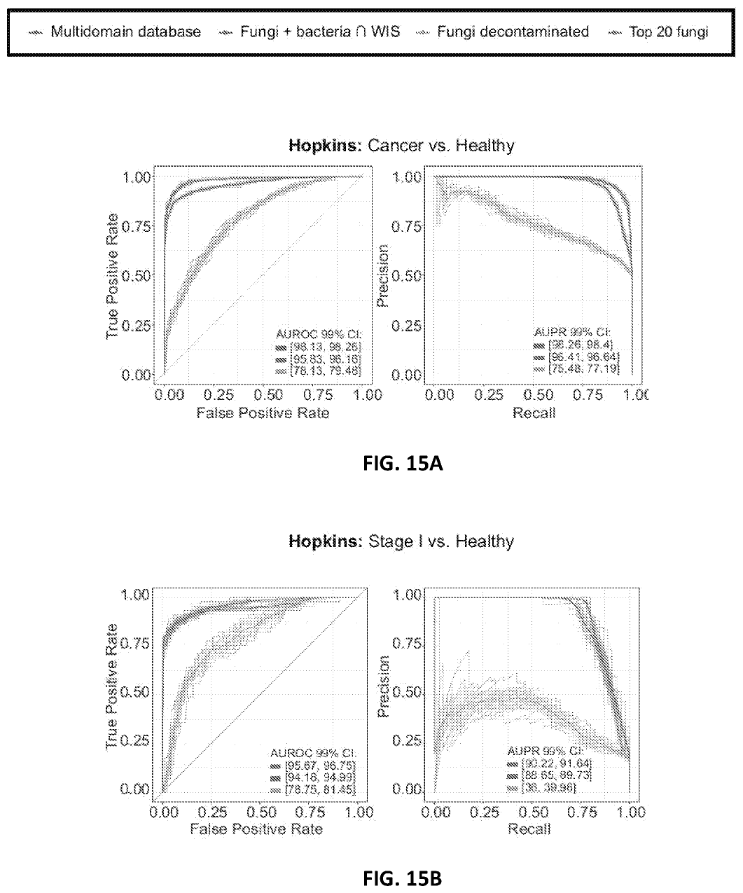

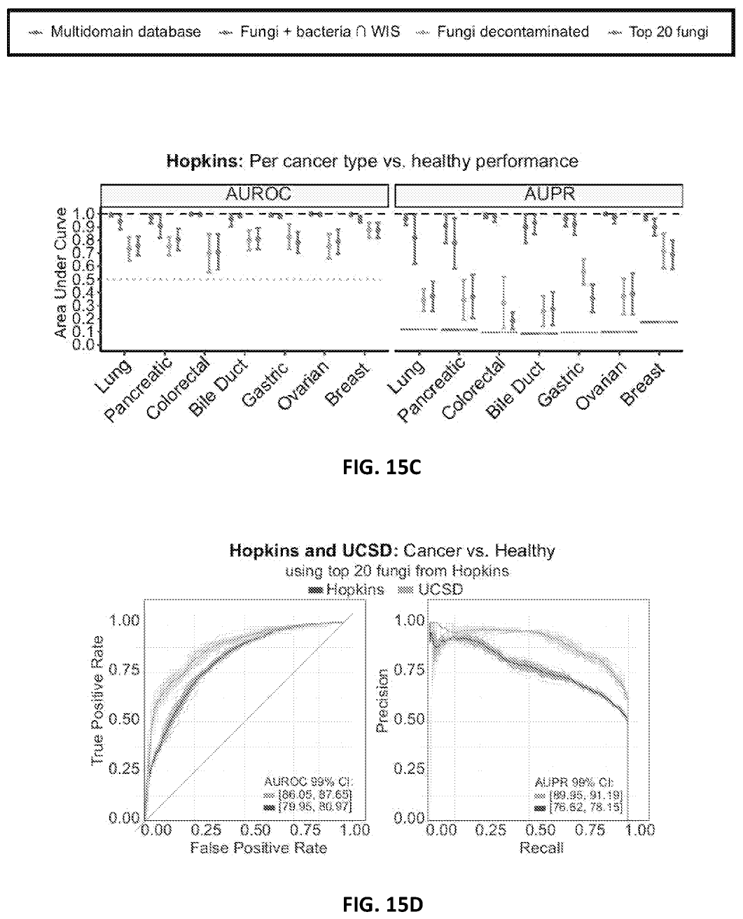

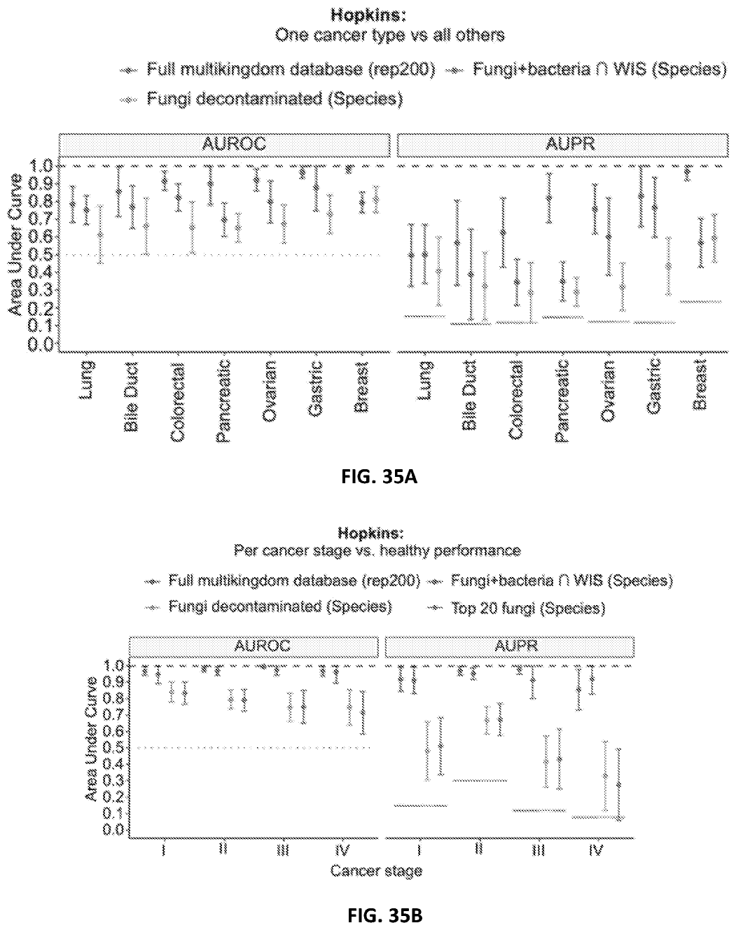

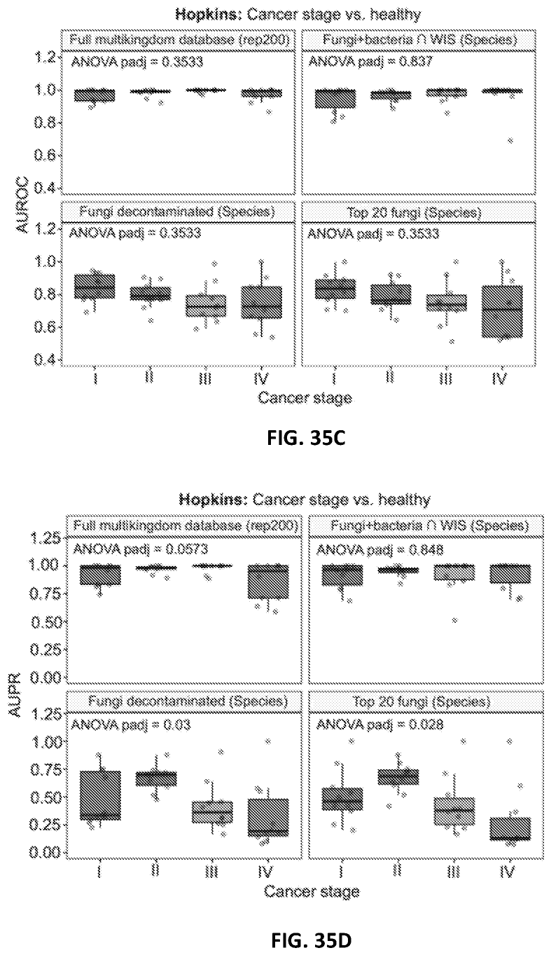

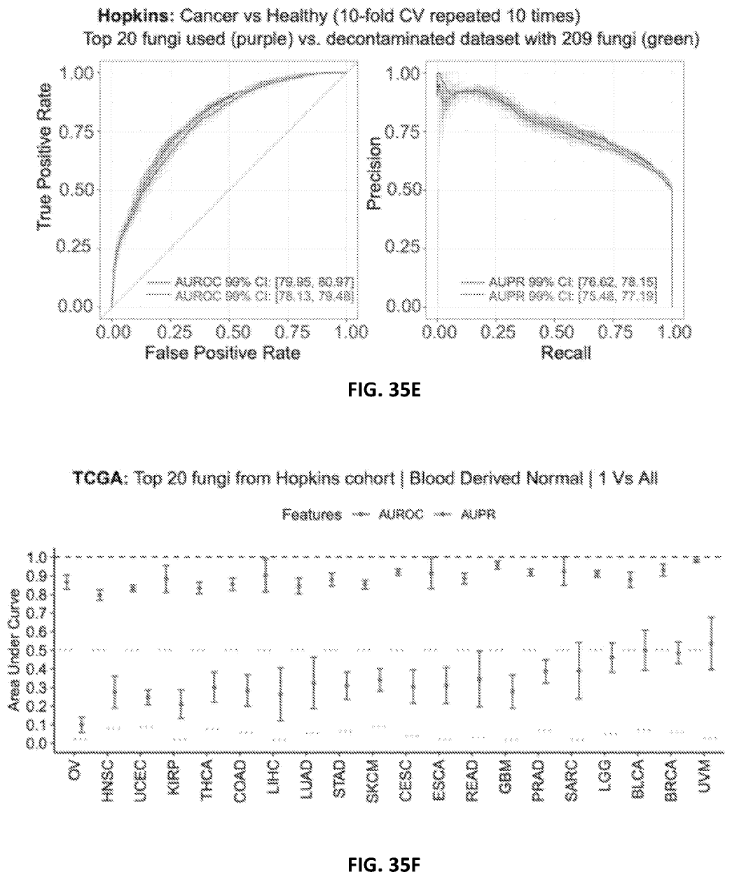

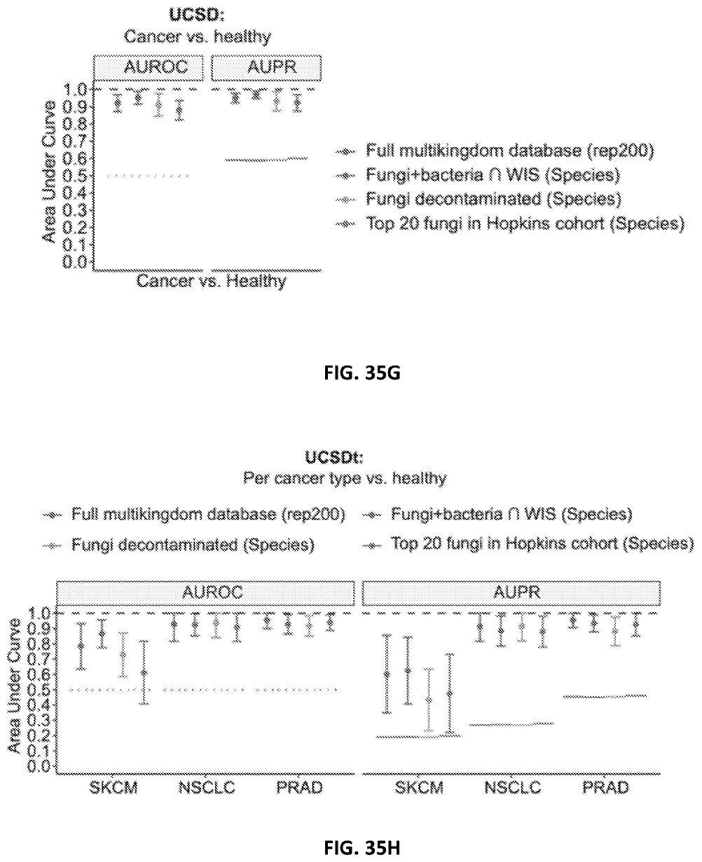

The Hopkins cohort underwent the same stringent human-read removal, microbial classification, and fungal decontamination as TCGA (n=537; 8 cancer types). Examining treatment-naive, earliest-timepoint samples (n=491), pan-cancer-versus-healthy diagnostic performance of raw microbial abundances using published ML framework and hyperparameters was estimated. Decontaminated fungal species (n=209) provided moderate discriminatory performance, and performance with multi-domain feature sets exceeded state-of-the-art, fragmentomic approaches (Avg. AUROCs: 96-98%), including a subset of 287 WIS tumor-overlapping fungi and bacteria (FIG. 15A). Running ML models with WIS-overlapping fungi, bacteria, or both also revealed significant, synergistic performances. Per cancer type ML versus controls performed similarly (FIG. 15C), with best fungal performance in breast cancer (AUROC 95% CI: [81.40, 93.53]%). Fungal discriminatory performance mostly plateaued at the taxonomic class level until species (FIG. 34A). Negative controls had null performances (FIG. 34B). All log-ratios of fungi from treatment-naive TCGA tumor mycotypes significantly varied among treatment-naive Hopkins cancer types in plasma (FIGS. 34C-34E), and the F1/F3 mycotype fungal log-ratio was significantly higher in cancer than controls (FIG. 34F). Testing ML models between cancer types also revealed moderate discrimination for decontaminated fungi and best performance with multi-domain features (FIG. 35A). Collectively, these analyses suggest clinical utility of plasma-derived, multi-domain microbial nucleic acids in treatment-naive patients.

ML analyses on Hopkins's 45 stage I, treatment-naive samples across eight cancer types versus healthy controls (FIG. 15B) was then conducted. Decontaminated fungal species provided notable performance, and multi-domain features matched or exceeded published fragmentomic approaches (Avg. AUROCs: 94-96%; FIG. 15B). ML across individual stages continued this pattern (FIG. 35B), with AUROCs not significantly varying across stages for any feature set (FIG. 35C) or AUPRs for multi-domain feature sets (FIG. 35D). These data suggest stage invariant performance of microbial-augmented liquid biopsies.

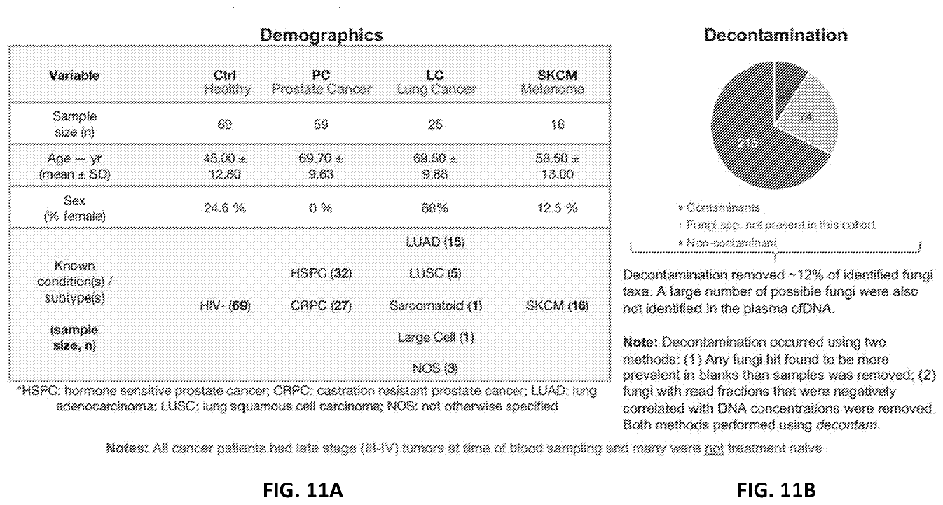

Hopkins pan-cancer versus healthy ML analyses revealed that the top 20 ranked, decontaminated fungal species (9.6% of total) performed at least as well as all 209 decontaminated fungi (FIG. 35E; Table 3). This reduced signature performed similarly to all decontaminated fungi in the Hopkins cohort when examining individual cancer types (FIG. 11C), stages (FIG. 35B), and negative controls (FIG. 34B). The 20 fungi also strongly discriminated among batch-corrected, pan-cancer TCGA blood samples (AUROC 95% Ci: [87.76, 89.79]%; FIG. 3F), collectively affirming a pan-cancer plasma mycobiome signature.

TABLE 3
Top 20 fungi in Hopkins cohort discriminating pan-cancer
vs. healthy in 10-fold CV repeated 10-times models

| Rank (based on feature | | | |
|---|---|---|---|
| importances) | OGU | species | reason_in_decontaminated_data |
| 1 | G000181695 | Malassezia globosa | Shared with WIS |
| 2 | G000006275 | Aspergillus flavus | In HMP gut mycobiome data |
| 3 | G003290485 | Malassezia restricta | Shared with WIS |
| 4 | G003025105 | Trichoderma asperellum | Known human association |
| 5 | G000182925 | Neurospora crassa | In HMP gut mycobiome data |
| 6 | G000303195 | Fusarium pseudograminearum | In HMP gut mycobiome data |
| 7 | G000149955 | Fusarium oxysporum | Shared with WIS |
| 8 | G000149685 | Lodderomyces elongisporus | In HMP gut mycobiome data |
| 9 | G000143535 | Botrytis cinerea | In HMP gut mycobiome data |
| 10 | G000230375 | Leptosphaeria maculans | In HMP gut mycobiome data |
| 11 | G001720205 | Cryptococcus amylolentus | Unknown human association but |
| | | | not predicted contaminant |
| 12 | G000027005 | Komagataella phaffii | In HMP gut mycobiome data |
| 13 | G001642055 | Alternaria alternata | Shared with WIS |
| 14 | G000225545 | Arthrobotrys oligospora | In HMP gut mycobiome data |
| 15 | G009193445 | Aspergillus pseudotamarii | Known human association |
| 16 | G000349005 | Pneumocystis murina | Known human association |
| 17 | G000263375 | Wallemia mellicola | In HMP gut mycobiome data |
| 18 | G000149925 | Puccinia graminis | In HMP gut mycobiome data |
| 19 | G002926055 | Candida haemuloni | Known human association |
| 20 | G001619985 | Xylona heveae | In HMP gut mycobiome data |

All 169 plasma samples from the UCSD cohort, which tested different experimental methods (fragmented vs. unfragmented DNA), patient types (treated vs. treatment-naive), and cancer types than the Hopkins cohort (1 of 8 Hopkins cancer types overlapped with UCSD) were then reprocessed. Although these differences limited direct comparisons, the Hopkins 20-fungi signature was tested to determine if the signature provided similar healthy-versus-cancer performance, which it did (Avg. AUROCs: 80-86%; FIG. 15D). The Hopkins 20-fungi signature performed similarly to the full set of UCSD decontaminated fungi in pan-cancer versus healthy (FIG. 35G) or per-cancer-type versus healthy comparisons (FIG. 35H), demonstrating its generalizability. Comparing performances with this signature or all decontaminated fungi in the UCSD cohort to negative controls revealed expected results (FIG. 36A). Log-ratios of TCGA-derived mycotype fungi did not significantly vary among UCSD cancer types, potentially due to treatment status, but ML between cancer types showed detectable differences (FIG. 36B). Like the Hopkins cohort, the F1/F3 mycotype fungal log-ratio was significantly higher in cancer versus healthy samples (FIG. 36C), highlighting their potential clinical utility. Exploratory analyses of immunotherapy response information on UCSD cohort patients also revealed that WIS-overlapping fungi moderately discriminated responders from non-responders in melanoma (FIG. 36D) but not lung cancer, although this remains to be validated in other cohorts. Overall, analyses across two independent cohorts and 10 cancer types show the utility of multi-domain cancer diagnostics and the plasma mycobiome, with a 20-fungi signature potentially able to distinguish pan-cancer versus healthy individuals.

### Example 2: Decontamination of Fungal Abundances

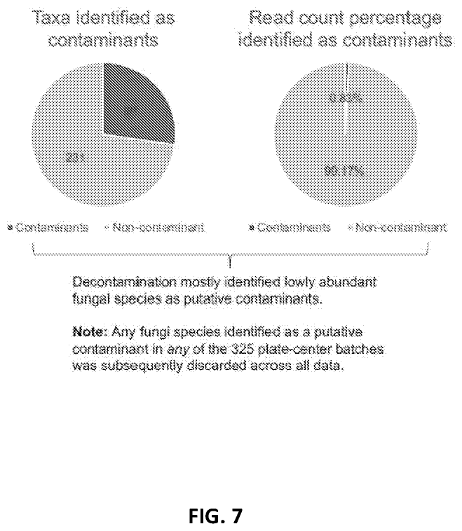

More than ten thousand biological samples were compared across 325 batches, defined as unique combinations of sequencing centers and their sequencing plates, to determine the presence and abundance of fungi. Contaminating fungi were determined by comparing the sample DNA or RNA concentrations with the fraction of reads assigned to each fungus across each batch, such that if a fungi was flagged as a contaminant in any individual batch, it was removed from all batches. After this decontamination, 231 non-contaminate fungal species remained and 67 putative contaminating fungal species were removed, as shown in FIG. 7. The contaminating fungal species accounted for 0.83% of read counts across all samples compared to the 99.17% of read counts that were not identified as being due to contaminants.

### Example 3: Batch Correction of Sequencing Reads Using Voom and SNM

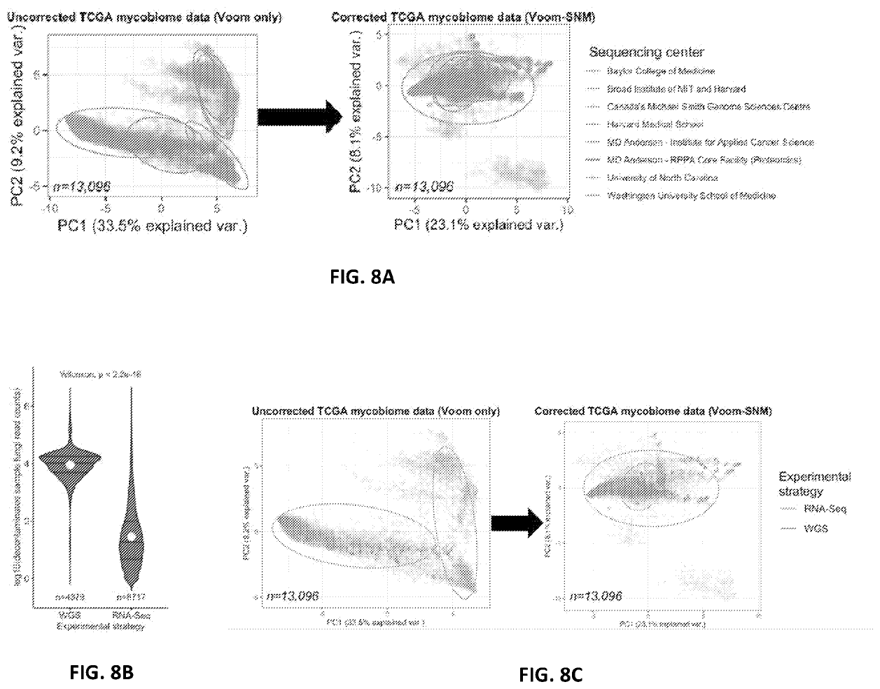

Batch correction methodologies such as Voom and SNM (PMID: 20363728, 24485249) were used with fungal abundances from TCGA samples across its various sequencing centers, as shown in FIGS. 8A-8C. Briefly, Voom converted discrete sequence counts to pseudo-normally distributed data, which was then used by SNM to iteratively remove batch effects in a supervised manner, such that biological signal is not removed while technical variation is removed, as shown in principal component plots shown in FIG. 8A and FIG. 8C. For example, FIG. 8A shows sequencing center-induced variation prior to Voom-SNM batch correction, and FIG. 8C shows experimental strategy (WGS vs. RNA-Seq) variation prior to Voom-SNM batch correction, each reflected by the post-batch correction overlap in the principal component plots. The difference in sequencing depth between the WGS and RNA-Seq samples, as shown in FIG. 8B, may have explained the original batch effects, which were corrected by Voom-SNM.

### Example 4: Identifying Disease Related Fungal Features of Blood Plasma

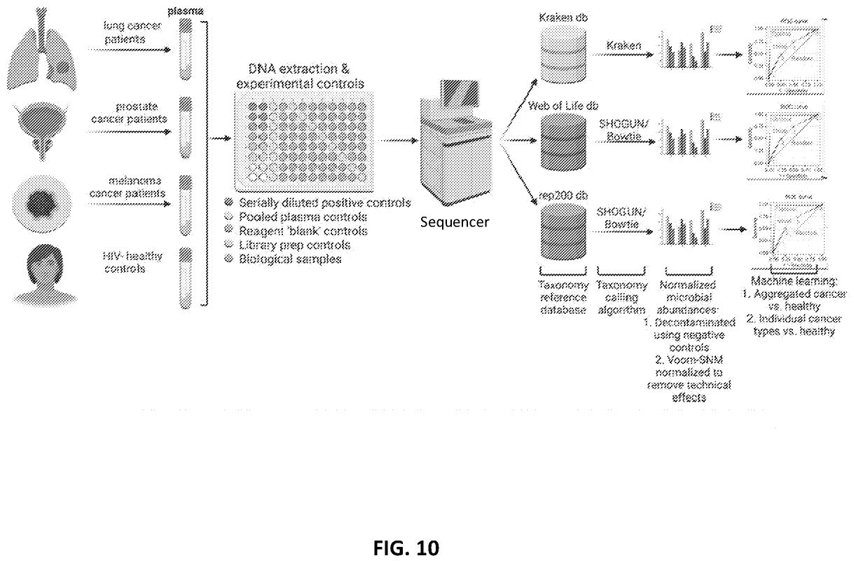

A biological sample of blood plasma may be used to determine one or more fungal and non-fungal presence and/or abundance features indicated of a disease or disorder (e.g., cancer) as described elsewhere herein, and as shown in FIG. 10. In this example, blood-derived plasma samples were extracted from patients with lung, prostate, and melanoma cancer, and HIV-healthy controls. Sequencing libraries, serially diluted positive controls, and negative “blank” experimental contamination controls were prepared and sequenced. The sequence reads were then aligned against a human reference genome library, as described elsewhere herein, and mapped to a non-human microbial taxonomy reference database (e.g., Web of Life database, PMID: 31792218; rep200) using various taxonomy calling algorithms (e.g., Kraken, SHOGUN, Bowtie2). The resulting mapped fungal and non-fungal microbial presence of the blood plasma were then decontaminated using the per-sample DNA concentrations (an in silico method) and the negative “blank” contamination controls, and then subjected to batch correction for age and sex differences between the groups using Voom-SNM. Results of the fungal decontamination and break down of each patient group is shown in FIGS. 11A-11B. The batch-corrected and decontaminated taxonomy features of the blood plasma were then used in combination with the corresponding disease information to generate one or more predictive models that are analyzed for their predictive accuracy.

### Example 5: Analyzing Percent Mapped and Number of Fungal and Non-Fungal Microbials Reads of TCGA Across Cancer Types

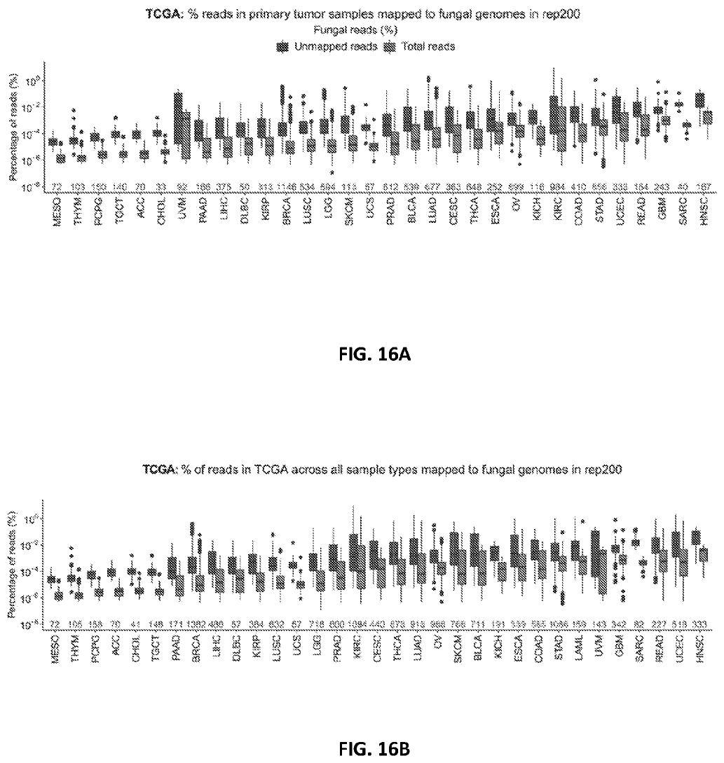

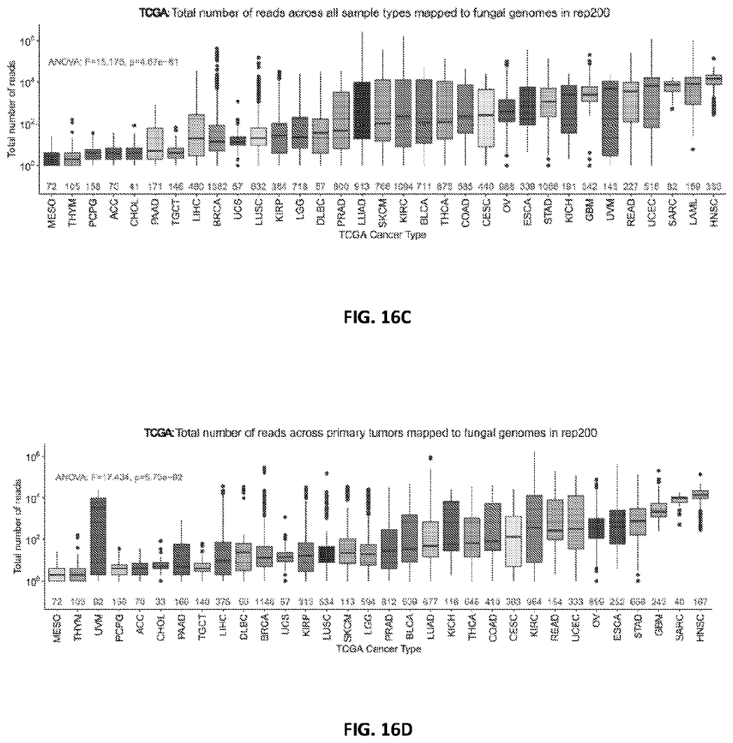

Biological sample sequencing read data from various cancer types was obtained from the TCGA for analysis for percent mapped reads to fungal, non-fungal microbial, and combined microbial genomes. Mapping of the TCGA sequencing reads was accomplished by methods described elsewhere herein (e.g., Kraken, SHOGUN, Bowtie2). The results of the analysis are shown in FIGS. 16A-16D and FIGS. 17A-17D. The percentage reads in primary tumor samples from TCGA that mapped to fungal genomes in the rep200 database were calculated and are shown in FIG. 16A. From FIG. 16A, one-way ANOVA results showed significant variation between cancer types for fungal percentages compared to unmapped (F=17.96, p=3.45×10⁻⁹⁵) and mapped (F=18.81, p=2.5×10⁻¹⁰⁰) sequencing reads. The percentage of reads in the TCGA across all sample types, including primary tumors and blood among other sample types, mapped to fungal genomes in the rep200 database were calculated and are shown in FIG. 16B. From FIG. 16B, one-way ANOVA results showed significant variation between cancer types for fungal percentages compared to unmapped (F=22.35, p=1.2×10⁻¹²⁶) and mapped (F=18.87, p=1.66×10⁻¹⁰⁴) sequencing reads. FIG. 16C and FIG. 16B show the total number of reads from the TCGA database across all sample types and primary tumors, respectively mapped to fungal genomes in the rep200 database, each with significant cancer type-varying distributions (inset on plots in FIG. 16C and FIG. 16B).

FIG. 17A shows percentage of reads in TCGA primary tumors mapped to all microbial genomes (i.e., fungal and non-fungal microbial) in the rep200 database versus unmapped (blue) and total (red) reads in the concomitant bam files. One-way ANOVA results showed significant variation between cancer types for pan-microbial percentages of unmapped (F=29.42, p=7.84×10⁻¹⁶⁵) and mapped (F=25.10, p=1.17×10⁻¹³⁸) sequencing reads.

FIG. 17B shows percentage of reads in TCGA across all sample types mapped to all microbial genomes in the rep200 database versus unmapped (blue) and total (red) reads in the concomitant bam files. One-way ANOVA results showed significant variation between cancer types for microbial percentages of unmapped (F=35.93, p=1.06×10⁻²¹²) and mapped (F=15.42, p=1.27×10⁻⁸²) sequencing reads.

FIG. 17C shows percentage of reads in TCGA primary tumors mapped to bacterial genomes in the rep200 database versus unmapped (blue) and total (red) reads in the concomitant bam files. One-way ANOVA results showed significant variation between cancer types for bacterial percentages of unmapped (F=26.74, p=1.31×10⁻¹⁴⁸) and mapped (F=25.56, p=1.84×10⁻¹⁴¹) sequencing reads.

FIG. 17D shows percentage of reads in TCGA across all sample types mapped to bacterial genomes in the rep200 database versus unmapped (blue) and total (red) reads in the concomitant bam files. One-way ANOVA results showed significant variation between cancer types for bacterial percentages of unmapped (F=31.32, p=1.29×10⁻¹⁸³) and mapped (F=15.25, p=1.50×10⁻⁸¹) sequencing reads.

What is claimed is:

1. A method of predicting cancer of a subject from a combined fungal and non-fungal microbial presence of a biological sample, comprising:
(a) detecting a fungal presence and a non-fungal microbial presence in a biological sample from a subject;
(b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and
(c) predicting a cancer of the subject by correlating the combined decontaminated fungal presence and the decontaminated non-fungal microbial presence of the subject to a known combined fungal presence and non-fungal microbial presence for one or more cancers.

2. The method of claim 1, wherein detecting comprises whole genome sequencing, shotgun sequencing, targeted sequencing, RNA sequencing, methylation sequencing, or any combination thereof.

3. The method as in claims 1 or 2, wherein the non-fungal microbial presence comprises bacteria, viruses, archaea, protists, or any combination thereof.

4. The method as in any of claims 1-3, wherein the non-fungal microbial presence comprises a non-fungal microbial abundance of the biological sample from the subject.

5. The method as in any of claims 1-4, wherein the fungal presence comprises a fungal abundance of the biological sample from the subject.

6. The method as in any of claims 1-5, wherein predicting the cancer further comprises predicting one or more cancers, one or more subtypes of cancer, the anatomic locations of one or more cancers, or any combination thereof in the subject.

7. The method as in any of claims 1-5, wherein predicting the cancer comprises predicting a stage of the cancer, cancer prognosis, a mutation status of the cancer, a future immunotherapy response of the cancer, an optimal therapy to treat the cancer, or any combination thereof for one or more subjects.

8. The method as in any of claims 1-5, wherein the cancer comprises a stage I or stage II cancer.

9. The method as in any of claims 1-5, wherein the predicting the cancer comprises simultaneously discriminating among one or more cancer types to diagnose a specific cancer type of the subject.

10. The method as in any of claims 1-9, wherein the cancer comprises bone, breast, lung, colon, brain, skin, ovary, pancreas, or any combination thereof type of cancer.

11. The method as in any of claims 1-9, wherein the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers.

12. The method as in any of claims 1-9, wherein cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

13. The method as in any of claims 1-12, wherein removing the contaminating non-fungal microbial features and the contaminating fungal features is completed by in silico decontamination.

14. The method as in any of claims 1-12, wherein removing the contaminating non-fungal microbial features and the contaminating fungal features is informed by experimental contamination controls.

15. The method as in any of claims 1-14, wherein predicting is conducted with a predictive model, wherein the predictive model comprises a machine learning model, regularized machine learning model, ensemble of machine learning models, or any combination thereof.

16. The method as in any of claims 1-15, wherein the predictive model comprises a random forest, neural network, naïve bayes, support vector machines, linear regression, k-nearest neighbors, k-means, decision tree, logistic regression, gradient boosting, or any combination thereof predictive model.

17. The method as in any of claims 1-16, wherein step (b) improves accuracy of the predictive model by at least 1%, at least 5%, at least 10%, at least 15%, or at least 20%.

18. The method as in any of claims 1-16, wherein step (b) is omitted.

19. The method as in any of claims 1-18, wherein the subject comprises anon-human mammal or a human subject.

20. The method as in any of claims 1-19, wherein the biological sample comprises a tissue sample, a liquid biopsy, whole blood biopsy, or any combination thereof samples.

21. The method as in any of claims 1-20, wherein the liquid biopsy comprises whole blood, red blood cells, plasma, white blood cells, saliva, urine, tears, breast milk, or any combination thereof.

22. The method of claim 20, wherein the whole blood biopsy comprises plasma, white blood cells, red blood cells, platelets, or any combination thereof.

23. The method as in any of claims 1-22, wherein the fungal presence comprises an abundance of fungal DNA, RNA, methylation, proteins, or any combination thereof.

24. The method as in any of claims 1-23, wherein the non-fungal microbial presence comprises an abundance of non-fungal microbial DNA, RNA, methylation, proteins, or any combination thereof.

25. The method as in any of claims 1-24, wherein detecting the fungal presence and the non-fungal microbial presence in the biological sample comprises:
(a) sequencing one or more nucleic acid molecules of the biological sample, thereby generating one or more sequencing reads;
(b) aligning the one or more sequencing reads to a reference human genome library and retaining one or more non-human sequencing reads that do not align to the reference human genome library; and
(c) mapping the one or more non-human sequencing reads to a fungal and non-fungal microbial reference genome library thereby generating a fungal presence and a non-fungal microbial presence of the biological sample.

26. The method as in any of claims 1-25, wherein aligning the one or more sequencing reads to a reference human genome library is omitted.

27. The method as in any of claims 1-26, wherein predicting further comprises predicting one or more anatomic locations of the cancer of the subject.

28. The method as in any of claims 1-27, wherein the predictive model is configured to receive the subject's biological sample cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof as an input to predict the cancer.

29. The method as in any of claims 1-28, wherein an area under a receiver operating curve of the predictive model is increased by at least 1%, at least 2%, at least 4%, at least 5%, or at least 10% when the combined decontaminated fungal presence and the decontaminated non-fungal presence is utilized during the correlation.

30. A method for training a predictive model based on fungal and non-fungal microbial features to diagnose cancer in a subject, comprising:
(a) receiving, from a biological sample of one or more subjects, a fungal presence, a non-fungal microbial presence, and a corresponding health state of the one or more subjects;
(b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and
(c) training a predictive model with the combined decontaminated fungal presence and the decontaminated non-fungal microbial presence, and the corresponding health state of the one or more subjects.

31. The method of claim 30, wherein the non-fungal microbial presence comprises a non-fungal microbial abundance of the biological sample from the one or more subjects.

32. The method as in claims 30 or 31, wherein the fungal presence comprises a fungal abundance of the biological sample from the one or more subjects.

33. The method as in any of claims 30-32, wherein the predictive model is configured to diagnose one or more cancers, one or more subtypes of cancer, one or more of the cancer's anatomic locations, or any combination thereof.

34. The method as in any of claims 30-32, wherein the predictive model is configured to predict a stage of cancer, cancer prognosis, a type of cancer at stage I or stage II, a mutation status of one or more cancers, a future immunotherapy response, an optimal therapy, or any combination thereof for one or more subjects.

35. The method as in any of claims 30-32, wherein the predictive model is configured to diagnose one or more stage I or stage II cancers in one or more subjects.

36. The method as in any of claims 30-32, wherein the predictive model is configured to simultaneously discriminate among one or more cancer types to diagnose a specific cancer type of the subject.

37. The method as in any of claims 30-36, wherein the associated type of cancer comprises bone, breast, lung, colon, brain, skin, ovary, pancreas, or any combination thereof type of cancer.

38. The method as in any of claims 30-37, wherein the predictive model is configured to diagnose adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers.

39. The method as in any of claims 30-37, wherein the predictive model is configured to diagnose one or more of the following cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

40. The method as in any of claims 30-39, wherein removing the contaminating non-fungal microbial features and the contaminating fungal features is completed by in silico decontamination.

41. The method as in any of claims 30-39, wherein removing the contaminating microbial features and the contaminating fungal features is informed by negative experimental controls.

42. The method as in any of claims 30-41, wherein the predictive model comprises a machine learning model, regularized machine learning model, ensemble of machine learning models, or any combination thereof.

43. The method as in any of claims 30-42, wherein the predictive model comprises a random forest, neural network, naïve bayes, support vector machines, linear regression, k-nearest neighbors, k-means, decision tree, logistic regression, gradient boosting, or any combination thereof predictive model.

44. The method as in any of claims 30-43, wherein step (b) improves accuracy of the predictive model by at least 1%, at least 5%, at least 10%, at least 15%, or at least 20%.

45. The method as in any of claims 30-43, wherein step (b) is omitted.

46. The method as in any of claims 30-45, wherein the one or more subjects comprise non-human mammal or human subjects.

47. The method as in any of claims 30-46, wherein the biological sample comprises a tissue sample, a liquid biopsy, whole blood biopsy, or any combination thereof samples.

48. The method as in any of claims 30-47, wherein the liquid biopsy comprises whole blood, red blood cells, plasma, white blood cells, saliva, urine, tears, breast milk, or any combination thereof.

49. The method of claim 47, wherein the whole blood biopsy comprises plasma, white blood cells, red blood cells, platelets, or any combination thereof.

50. The method as in any of claims 30-49, wherein the fungal presence comprises an abundance of fungal DNA, RNA, methylation, proteins, or any combination thereof.

51. The method as in any of claims 30-50, wherein the non-fungal microbial presence comprises an abundance of non-fungal microbial DNA, RNA, methylation, proteins, or any combination thereof.

52. The method as in any of claims 30-51, wherein detecting the fungal presence and the non-fungal microbial presence in the biological sample comprises:
(a) sequencing one or more nucleic acid molecules of the biological sample, thereby generating one or more sequencing reads;
(b) aligning the one or more sequencing reads to a reference human genome library and retain one or more non-human sequencing reads that do not align to the reference human genome library; and
(c) mapping the one or more non-human sequencing reads to a fungal and non-fungal microbial reference genome library thereby generating a fungal presence and a non-fungal microbial presence of the biological sample.

53. The method as in any of claims 30-52, wherein aligning the one or more sequencing reads to a reference human genome library is omitted.

54. The method as in any of claims 30-52, wherein the predictive model is configured to predict one or more anatomic locations of a cancer of a subject by providing the trained predictive model an input of a non-fungal microbial presence and a fungal presence of the subject's biological sample.

55. The method as in any of claims 30-54, wherein the predictive model is further trained with cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof.

56. The method as in any of claims 30-55, wherein receiving comprises whole genome sequencing, shotgun sequencing, targeted sequencing, RNA sequencing, methylation sequencing, or any combination thereof sequencing of the fungal and non-fungal microbial presence nucleic acid molecules in the biological sample.

57. The method as in any of claims 30-56, wherein the health state of the one or more subjects comprises a non-cancerous health state or cancerous health state.

58. The method as in any of claims 30-57, wherein the non-cancerous health state comprises a non-cancerous disease health state or a non-diseased health state

59. A method for training a predictive model based on fungal and non-fungal microbial features to predict cancer in a subject, comprising:
(a) receiving a fungal presence, a non-fungal microbial presence, and a health state of one or more subjects from a database;
(b) removing contaminating fungal features of the fungal presence and contaminating non-fungal microbial features the non-fungal microbial presence while retaining decontaminated fungal features and decontaminated non-fungal microbial features, thereby producing a combined decontaminated fungal presence and a decontaminated non-fungal microbial presence; and
(c) training a predictive model configured to predict cancer in a subject with the combined decontaminated fungal presence and decontaminated non-fungal microbial presence, and the corresponding health state of the one or more subjects.

60. The method of claim 59, wherein the non-fungal microbial presence comprises a non-fungal microbial abundance of the biological sample from the one or more subjects.

61. The method as in claims 59 or 60, wherein the fungal presence comprises a fungal abundance of the biological sample from the one or more subjects.

62. The method as in any of claims 59-61, wherein the predictive model is configured to diagnose one or more cancers, one or more subtypes of cancer, one or more of its anatomic locations, or any combination thereof.

63. The method as in any of claims 59-61, wherein the predictive model is configured to predict a stage of cancer, a cancer prognosis, a type of cancer at stage I or stage II, a mutation status of one or more cancers, a future immunotherapy response, an optimal therapy, or any combination thereof for one or more subjects.

64. The method as in any of claims 59-61, wherein the predictive model is configured to diagnose one or more stage I or stage II cancers in one or more subjects.

65. The method as in any of claims 59-61, wherein the predictive model is configured to simultaneously discriminate among one or more cancer types to diagnose a specific cancer type of the subject.

66. The method as in any of claims 59-65, wherein the associated type of cancer comprises bone, breast, lung, colon, brain, skin, ovary, pancreas, or any combination thereof type of cancer.

67. The method as in any of claims 59-66, wherein the predictive model is configured to diagnose adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers.

68. The method as in any of claims 59-66, wherein the predictive model is configured to diagnose one or more of the following cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

69. The method as in any of claims 59-68, wherein removing the contaminating non-fungal microbial features and the contaminating fungal features is completed by in silico decontamination.

70. The method as in any of claims 59-68, wherein removing the contaminating non-fungal microbial features and the contaminating fungal features is informed by experimental controls.

71. The method as in any of claims 59-70, wherein the predictive model comprises a machine learning model, regularized machine learning model, ensemble of machine learning models, or any combination thereof.

72. The method as in any of claims 59-71, wherein the predictive model comprises a random forest, neural network, naïve bayes, support vector machines, linear regression, k-nearest neighbors, k-means, decision tree, logistic regression, gradient boosting, or any combination thereof predictive model.

73. The method as in any of claims 59-72, wherein step (b) improves accuracy of the predictive model by at least 1%, at least 5%, at least 10%, at least 15%, or at least 20%.

74. The method as in any of claims 59-72, wherein step (b) is omitted.

75. The method as in any of claims 59-74, wherein the one or more subjects comprise non-human mammal or human subjects.

76. The method as in any of claims 59-75, wherein the biological sample comprises a tissue sample, a liquid biopsy, whole blood biopsy, or any combination thereof samples.

77. The method as in any of claims 59-76, wherein the liquid biopsy comprises whole blood, red blood cells, plasma, white blood cells, saliva, urine, tears, breast milk, or any combination thereof.

78. The method of claim 76, wherein the whole blood biopsy comprises plasma, white blood cells, red blood cells, platelets, or any combination thereof.

79. The method as in any of claims 59-78, wherein the fungal presence comprises an abundance of fungal DNA, RNA, methylation, proteins, or any combination thereof.

80. The method as in any of claims 59-79, wherein the non-fungal microbial presence comprises an abundance of non-fungal microbial DNA, RNA, methylation, proteins, or any combination thereof.

81. The method as in any of claims 59-80, wherein detecting the fungal presence and the non-fungal microbial presence in the biological sample comprises:
(a) sequencing one or more nucleic acid molecules of the biological sample, thereby generating one or more sequencing reads;
(b) aligning the one or more sequencing reads to a reference human genome library and retain one or more non-human sequencing reads that do not align to the reference human genome library; and
(c) mapping the one or more non-human sequencing reads to a fungal and non-fungal microbial reference genome library thereby generating a fungal presence and a non-fungal microbial presence of the biological sample.

82. The method as in any of claims 59-81, wherein aligning the one or more sequencing reads to a reference human genome library is omitted.

83. The method as in any of claims 59-81, wherein predictive model is configured to predict an anatomic location of a cancer of a subject by providing the trained predictive model an input of a non-fungal microbial presence and a fungal presence of the subject's biological sample.

84. The method as in any of claims 59-83, wherein the predictive model is further trained with cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof.

85. The method as in any of claims 59-84, wherein detecting comprises whole genome sequencing, shotgun sequencing, targeted sequencing, RNA sequencing, methylation sequencing, or any combination thereof sequencing of the fungal and non-fungal microbial presence nucleic acid molecules.

86. The method as in any of claims 59-85, wherein the database comprises The Cancer Genome Atlas database (TCGA), the International Cancer Genome Consortium (ICGC) database, the Pan-Cancer Atlas of Whole Genomes (PCAWG) database, the Therapeutically Applicable Research to Generate Effective Treatments (TARGET) database, the Clinical Proteomic Tumor Analysis Consortium (CPTAC) database, the Hartwig Medical Foundation (HMF) metastasis database, the Tracking Non-Small-Cell Lung Cancer Evolution through Therapy (TRACERx) database, the 100,000 Genomes Project, or any combination thereof.

87. The method as in any of claims 59-86, wherein the health state of the one or more subjects comprises a non-cancerous health state or cancerous health state.

88. The method as in any of claims 59-87, wherein the non-cancerous health state comprises a non-cancerous diseased health state or a non-diseased health state

89. A method of treating cancer of a subject based on a combined microbial and fungal presence of a biological sample of the subject, comprising:
(a) detecting a fungal presence and a non-fungal microbial presence in a biological sample from a subject;
(b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and
(c) administering a therapeutic to treat a cancer of the subject determined by at least a correlation between the combined decontaminated fungal presence and the decontaminated non-fungal microbial presence of the subject to a known combined fungal presence and non-fungal microbial presence of subjects with cancer treated with the therapeutic.

90. The method of claim 89, wherein the non-fungal microbial presence comprises a non-fungal microbial abundance of the biological sample from the one or more subjects.

91. The method as in claims 89 or 90, wherein the fungal presence comprises a fungal abundance of the biological sample from the one or more subjects.

92. The method as in any of claims 89-91, wherein the cancer of the comprises one or more cancers, one or more subtypes of cancer, or any combination thereof.

93. The method as in any of claims 89-91, wherein the cancer comprises a stage I or stage II cancer.

94. The method as in any of claims 89-93, wherein the cancer comprises bone, breast, lung, colon, brain, skin, ovary, pancreas, or any combination thereof type of cancer.

95. The method as in any of claims 89-94, wherein the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers.

96. The method as in any of claims 89-94, wherein the cancer comprises a cancer type outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

97. The method as in any of claims 89-96, wherein removing the contaminating non-fungal microbial features and the contaminating fungal features is completed by in silico decontamination.

98. The method as in any of claims 89-96, wherein removing the contaminating non-fungal microbial features and the contaminating fungal features is completed by experimental controls.

99. The method as in any of claims 89-98, wherein the correlation is determined by a predictive model, wherein the predictive model comprises a machine learning model, regularized machine learning model, ensemble of machine learning models, or any combination thereof.

100. The method as in any of claims 89-99, wherein the predictive model comprises a random forest, neural network, naïve bayes, support vector machines, linear regression, k-nearest neighbors, k-means, decision tree, logistic regression, gradient boosting, or any combination thereof predictive model.

101. The method as in any of claims 89-100, wherein step (b) improves accuracy of the predictive model by at least 1%, at least 5%, at least 10%, at least 15%, or at least 20%.

102. The method as in any of claims 89-100, wherein step (b) is omitted.

103. The method as in any of claims 89-102, wherein the subject comprises a non-human mammal or human subject.

104. The method as in any of claims 89-103, wherein the biological sample comprises a tissue sample, a liquid biopsy, whole blood biopsy, or any combination thereof samples.

105. The method as in any of claims 89-104, wherein the liquid biopsy comprises whole blood, red blood cells, plasma, white blood cells, saliva, urine, tears, breast milk, or any combination thereof.

106. The method of claim 104, wherein the whole blood biopsy comprises plasma, white blood cells, red blood cells, platelets, or any combination thereof.

107. The method as in any of claims 89-106, wherein the fungal presence comprises an abundance of fungal DNA, RNA, methylation, proteins, or any combination thereof.

108. The method as in any of claims 89-107, wherein the non-fungal microbial presence comprises an abundance of non-fungal microbial DNA, RNA, methylation, proteins, or any combination thereof.

109. The method as in any of claims 89-108, wherein detecting the fungal presence and the non-fungal microbial presence in the biological sample comprises:
(a) sequencing one or more nucleic acid molecules of the biological sample, thereby generating one or more sequencing reads;
(b) aligning the one or more sequencing reads to a reference human genome library and retain one or more non-human sequencing reads that do not align to the reference human genome library; and
(c) mapping the one or more non-human sequencing reads to a fungal and non-fungal microbial reference genome library thereby generating a fungal presence and a non-fungal microbial presence of the biological sample.

110. The method as in any of claims 89-109, wherein the predictive model is trained with one or more biologic samples from one or more subjects comprising a decontaminated fungal presence, decontaminated non-fungal microbial presence cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof, to diagnose a corresponding subject's cancer, inform an optimal treatment to treat the subject's cancer, or any combination thereof.

111. The method as in any of claims 89-110, wherein detecting comprises whole genome sequencing, shotgun sequencing, targeted sequencing, RNA sequencing, methylation sequencing, or any combination thereof sequencing of the fungal and non-fungal microbial presence nucleic acid molecules in the biological sample.

112. The method as in any of claims 89-111, wherein the treatment repurposes an existing medication, which may or may not have been originally approved for targeting cancer.

113. The method as in any of claims 89-112, wherein the treatment comprises a small molecule, a biologic, a probiotic, a virus, a bacteriophage, an immunotherapy, a broad spectrum antibiotic, or any combination thereof.

114. The method as in any of claims 89-113, wherein the probiotic comprises an engineered bacterium strain or ensemble of engineered bacteria.

115. The method as in any of claims 89-112, wherein the treatment comprises an adjuvant given in combination with a primary treatment against the cancer to improve the efficacy of the primary treatment.

116. The method as in any of claims 89-112, wherein the treatment comprises adoptive cell transfer to target microbial antigens associated with the cancer or cancer microenvironment.

117. The method as in any of claims 89-112, wherein the treatment comprises a cancer vaccine that exploits microbial antigens associated with the cancer or cancer microenvironment.

118. The method as in any of claims 89-112, wherein the treatment comprises a monoclonal antibody against microbial antigens associated with the cancer or cancer microenvironment.

119. The method as in any of claims 89-112, wherein the treatment comprises an antibody-drug conjugate designed to at least partially target microbial antigens associated with the cancer or cancer microenvironment.

120. The method as in any of claims 89-112, wherein the treatment comprises a multi-valent antibody, antibody fragment, or antibody derivative thereof designed to at least partially target one or more microbial antigens associated with the cancer or cancer microenvironment.

121. The method as in any of claims 89-112, wherein the treatment comprises a targeted antibiotic against a particular kind of microbe or class of functionally or biologically similar microbes.

122. The method as in any of claims 89-112, wherein two or more of the following treatment types are combined such that at least one type exploits the cancer microbial presence or abundance to enhance overall therapeutic efficacy: small molecules, biologics, engineered host-derived cell types, probiotics, engineered bacteria, natural-but-selective viruses, engineered viruses, and bacteriophages.

123. A computer-implemented method for utilizing a predictive model to predict cancer of a subject from a combined fungal and non-fungal microbial presence of a biological sample, comprising:
(a) detecting a fungal presence and a non-fungal microbial presence in a biological sample from a subject;
(b) removing contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and
(c) predicting, using a computer that implements the predictive model, a cancer of the subject by correlating the combined decontaminated fungal presence and the decontaminated non-fungal microbial presence of the subject to a known combined fungal presence and non-fungal microbial presence for one or more cancers.

124. The computer-implemented method of claim 123, wherein detecting comprises whole genome sequencing, shotgun sequencing, targeted sequencing, RNA sequencing, methylation sequencing, or any combination thereof.

125. The computer-implemented method as in claims 123 or 124, wherein the non-fungal microbial presence comprises bacteria, viruses, archaea, protists, or any combination thereof.

126. The computer-implemented method as in any of claims 123-125, wherein the non-fungal microbial presence comprises a non-fungal microbial abundance of the biological sample from the subject.

127. The computer-implemented method as in any of claims 123-126, wherein the fungal presence comprises a fungal abundance of the biological sample from the subject.

128. The computer-implemented method as in any of claims 123-127, wherein predicting the cancer further comprises predicting one or more cancers, one or more subtypes of cancer, the anatomic locations of one or more cancers, or any combination thereof in the subject.

129. The computer-implemented method as in any of claims 123-127, wherein predicting the cancer comprises predicting a stage of the cancer, cancer prognosis, a mutation status of the cancer, a future immunotherapy response of the cancer, an optimal therapy to treat the cancer, or any combination thereof for one or more subjects.

130. The computer-implemented method as in any of claims 123-127, wherein the cancer comprises a stage I or stage II cancer.

131. The computer-implemented method as in any of claims 123-127, wherein predicting the cancer comprises simultaneously discriminating among one or more cancer types to diagnose a specific cancer type of the subject.

132. The computer-implemented method as in any of claims 123-131, wherein the cancer comprises bone, breast, lung, colon, brain, skin, ovary, pancreas, or any combination thereof type of cancer.

133. The computer-implemented method as in any of claims 123-132, wherein the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers.

134. The computer-implemented method as in any of claims 123-132, wherein cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

135. The computer-implemented method as in any of claims 123-134, wherein removing the contaminating non-fungal microbial features and the contaminating fungal features is completed by in silico decontamination.

136. The computer-implemented method as in any of claims 123-134, wherein removing the contaminating non-fungal microbial features and the contaminating fungal features is informed by experimental contamination controls.

137. The computer-implemented method as in any of claims 123-136, wherein the predictive model comprises a machine learning model, regularized machine learning model, ensemble of machine learning models, or any combination thereof.

138. The computer-implemented method as in any of claims 123-137, wherein the predictive model comprises a random forest, neural network, naïve bayes, support vector machines, linear regression, k-nearest neighbors, k-means, decision tree, logistic regression, gradient boosting, or any combination thereof predictive model.

139. The computer-implemented method as in any of claims 123-138, wherein step (b) improves accuracy of the predictive model by at least 1%, at least 5%, at least 10%, at least 15%, or at least 20%.

140. The computer-implemented method as in any of claims 123-139, wherein step (b) is omitted.

141. The computer-implemented method as in any of claims 123-140, wherein the subject comprises a non-human mammal or a human subject.

142. The computer-implemented method as in any of claims 123-141, wherein the biological sample comprises a tissue sample, a liquid biopsy, whole blood biopsy, or any combination thereof samples.

143. The computer-implemented method as in any of claims 123-142, wherein the liquid biopsy comprises whole blood, red blood cells, plasma, white blood cells, saliva, urine, tears, breast milk, or any combination thereof.

144. The computer-implemented method as in any of claims 123-143, wherein the whole blood biopsy comprises plasma, white blood cells, red blood cells, platelets, or any combination thereof.

145. The computer-implemented method as in any of claims 123-144, wherein the fungal presence comprises an abundance of fungal DNA, RNA, methylation, proteins, or any combination thereof.

146. The computer-implemented method as in any of claims 123-145, wherein the non-fungal microbial presence comprises an abundance of non-fungal microbial DNA, RNA, methylation, proteins, or any combination thereof.

147. The computer-implemented method as in any of claims 123-146, wherein detecting the fungal presence and the non-fungal microbial presence in the biological sample comprises:
(a) sequencing one or more nucleic acid molecules of the biological sample, thereby generating one or more sequencing reads;
(b) aligning the one or more sequencing reads to a reference human genome library and retaining one or more non-human sequencing reads that do not align to the reference human genome library; and
(c) mapping the one or more non-human sequencing reads to a fungal and non-fungal microbial reference genome library thereby generating a fungal presence and a non-fungal microbial presence of the biological sample.

148. The computer-implemented method as in any of claims 123-147, wherein aligning the one or more sequencing reads to a reference human genome library is omitted.

149. The computer-implemented method as in any of claims 123-148, wherein predicting further comprises predicting one or more anatomic locations of the cancer of the subject.

150. The computer-implemented method as in any of claims 123-149, wherein the predictive model is further configured to receive the subject's biological sample cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof as an input to predict the cancer.

151. The computer-implemented method as in any of claims 123-150, wherein detecting comprises whole genome sequencing, shotgun sequencing, targeted sequencing, RNA sequencing, methylation sequencing, or any combination thereof the one or more nucleic acid molecules of the biological sample.

152. The computer-implemented method as in any of claims 123-151, wherein an area under a receiver operating curve of the predictive model for predicting the cancer of the subject is increased by at least 1%, at least 2%, at least 4%, at least 5%, or at least 10% when the combined decontaminated fungal presence and the decontaminated non-fungal presence is utilized during the correlation.

153. A computer system configured to predict cancer of a subject from a combined fungal and non-fungal microbial presence of a biological sample, comprising:
(a) one or more processors; and
(b) a non-transient computer readable storage medium including software, wherein the software comprises executable instructions that, as a result of the execution, cause the one or more processors of the computer system to:
(i) detect a fungal presence and a non-fungal microbial presence in a biological sample from a subject;
(ii) remove contaminating fungal features of the fungal presence and contaminating non-fungal microbial features of the non-fungal microbial presence while retaining decontaminated fungal features and decontaminated non-fungal microbial features, thereby producing a combined decontaminated fungal presence and a decontaminated non-fungal microbial presence; and
(iii) predict a cancer of the subject by correlating the combined decontaminated fungal presence and the decontaminated non-fungal microbial presence of the subject to a known combined fungal presence and non-fungal microbial presence for one or more cancers.

154. The computer system of claim 153, wherein detecting comprises whole genome sequencing, shotgun sequencing, targeted sequencing, RNA sequencing, methylation sequencing, or any combination thereof.

155. The computer system as in claims 153 or 154, wherein the non-fungal microbial presence comprises bacteria, viruses, archaea, protists, or any combination thereof.

156. The computer system as in any of claims 153-155, wherein the non-fungal microbial presence comprises a non-fungal microbial abundance of the biological sample from the subject.

157. The computer system as in any of claims 153-156, wherein the fungal presence comprises a fungal abundance of the biological sample from the subject.

158. The computer system as in any of claims 153-157, wherein predicting the cancer further comprises predicting one or more cancers, one or more subtypes of cancer, the anatomic locations of one or more cancers, or any combination thereof in the subject.

159. The computer system as in any of claims 153-157, wherein predicting the cancer comprises predicting a stage of the cancer, cancer prognosis, a mutation status of the cancer, a future immunotherapy response of the cancer, an optimal therapy to treat the cancer, or any combination thereof for one or more subjects.

160. The computer system as in any of claims 153-157, wherein the cancer comprises a stage I or stage II cancer.

161. The computer system as in any of claims 153-157, wherein the predicting the cancer comprises simultaneously discriminating among one or more cancer types to diagnose a specific cancer type of the subject.

162. The computer system as in any of claims 153-161, wherein the cancer comprises bone, breast, lung, colon, brain, skin, ovary, pancreas, or any combination thereof type of cancer.

163. The computer system as in any of claims 153-161, wherein the cancer comprises adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, duodenal cancer, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof types of cancers.

164. The computer system as in any of claims 153-161, wherein cancer comprises one or more cancer types outside the intestine: adrenocortical carcinoma, bladder urothelial carcinoma, brain lower grade glioma, breast invasive carcinoma, cervical squamous cell carcinoma and endocervical adenocarcinoma, glioblastoma multiforme, head and neck squamous cell carcinoma, kidney chromophobe, kidney renal clear cell carcinoma, kidney renal papillary cell carcinoma, lung adenocarcinoma, lung squamous cell carcinoma, lymphoid neoplasm diffuse large B-cell lymphoma, mesothelioma, ovarian serous cystadenocarcinoma, pheochromocytoma and paraganglioma, prostate adenocarcinoma, sarcoma, skin cutaneous melanoma, testicular germ cell tumors, thymoma, thyroid carcinoma, uterine carcinosarcoma, uterine corpus endometrial carcinoma, uveal melanoma, or any combination thereof types of cancers.

165. The computer system as in any of claims 153-164, wherein removing the contaminating non-fungal microbial features and the contaminating fungal features is completed by in silico decontamination.

166. The computer system as in any of claims 153-164, wherein removing the contaminating non-fungal microbial features and the contaminating fungal features is informed by experimental contamination controls.

167. The computer system as in any of claims 153-166, wherein the predictive model comprises a machine learning model, regularized machine learning model, ensemble of machine learning models, or any combination thereof.

168. The computer system as in any of claims 153-167, wherein the predictive model comprises a random forest, neural network, naïve bayes, support vector machines, linear regression, k-nearest neighbors, k-means, decision tree, logistic regression, gradient boosting, or any combination thereof predictive model.

169. The computer system as in any of claims 153-168, wherein step (b) improves accuracy of the predictive model by at least 1%, at least 5%, at least 10%, at least 15%, or at least 20%.

170. The computer system as in any of claims 153-168, wherein step (b) is omitted.

171. The computer system as in any of claims 153-170, wherein the subject comprises a non-human mammal or a human subject.

172. The computer system as in any of claims 153-171, wherein the biological sample comprises a tissue sample, a liquid biopsy, whole blood biopsy, or any combination thereof samples.

173. The computer system as in any of claims 153-172, wherein the liquid biopsy comprises whole blood, red blood cells, plasma, white blood cells, saliva, urine, tears, breast milk, or any combination thereof.

174. The computer system as in any of claims 153-173, wherein the whole blood biopsy comprises plasma, white blood cells, red blood cells, platelets, or any combination thereof.

175. The computer system as in any of claims 153-174, wherein the fungal presence comprises an abundance of fungal DNA, RNA, methylation, proteins, or any combination thereof.

176. The computer system as in any of claims 153-175, wherein the non-fungal microbial presence comprises an abundance of non-fungal microbial DNA, RNA, methylation, proteins, or any combination thereof.

177. The computer system as in any of claims 153-176, wherein detecting the fungal presence and the non-fungal microbial presence in the biological sample comprises:
(a) sequencing one or more nucleic acid molecules of the biological sample, thereby generating one or more sequencing reads;
(b) aligning the one or more sequencing reads to a reference human genome library and retaining one or more non-human sequencing reads that do not align to the reference human genome library; and
(c) mapping the one or more non-human sequencing reads to a fungal and non-fungal microbial reference genome library thereby generating a fungal presence and a non-fungal microbial presence of the biological sample.

178. The computer system as in any of claims 153-177, wherein aligning the one or more sequencing reads to a reference human genome library is omitted.

179. The computer system as in any of claims 153-178, wherein predicting further comprises predicting one or more anatomic locations of the cancer of the subject.

180. The computer system as in any of claims 153-179, wherein the predictive model is further configured to receive the subject's biological sample cell-free tumor DNA, cell-free tumor RNA, exosomal-derived tumor DNA, exosomal-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, blood-derived protein concentrations, plasma-derived protein concentrations, or any combination thereof as an input to predict the cancer.

181. The computer system as in any of claims 153-180, wherein detecting comprises whole genome sequencing, shotgun sequencing, targeted sequencing, RNA sequencing, methylation sequencing, or any combination thereof the one or more nucleic acid molecules of the biological sample.

182. The computer system as in any of claims 153-181, wherein an area under a receiver operating curve of the predictive model for predicting the cancer of the subject is increased by at least 1%, at least 2%, at least 4%, at least 5%, or at least 10% when the combined decontaminated fungal presence and the decontaminated non-fungal presence is utilized during the correlation.
